SEQUENCE LISTING (50 sequences)

SEQ ID NO: 1 - DNA, 1233 bp, Zea mays
atggccatcatactcgtacgagcagcgtcgccggggctctccgccgccgacagcatcagccaccaggggactctccagtgctccaccctgctcaagacgaagaggccggcggcccgccggtggatgccctgctcgctccttggcctccacccgtgggaggctggccgtccctcccccgccgtctactccagcctccccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctag

SEQ ID NO: 2 - DNA, 1479 bp, Erwinia uredovora
atgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttga

SEQ ID NO: 3 - DNA, 1176 bp, Escherichia coli
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgcgtttacaacaagctgtaa

SEQ ID NO: 4 - DNA, 2154 bp, Arabidopsis thaliana
atggcttcttctgcatttgctttcccctcttatataatcaccaaaggtggtttgtctactgactcatgtaagagtacttcacttagttcatccagatcattggttactgatttgcctagtccatgcctgaaacccaataacaacagtcattcaaatcgtcgagctaaggtatgtgcaagcctcgcggaaaagggagaatactattctaatcgccctccaacacctctcttagacacaatcaactaccctatacatatgaaaaatttatctgtaaaagagttgaagcaactatcggatgagctgagatccgatgtcattttcaacgtcagcaagaccgggggtcatcttggatctagtctaggcgttgtggagctgactgtcgctctccactacattttcaataccccgcaggataaaattttgtgggacgttgggcaccaatcatatcctcataagatccttacaggtagaagaggtaagatgccgaccatgagacaaactaacgggctttccggatttacaaagcggggagagagcgagcacgattgctttggaactggtcattcatcaacaactatcagtgccgggctcggtatggctgtgggtagagacttgaagggaaaaaataacaacgttgtggcagtaatcggtgatggtgcgatgacagccggccaggcctatgaagctatgaataatgccggatacttagacagcgatatgatcgtaatactcaacgacaacaaacaggtgtcccttccaacagcgactctagacggcccttcgccgccagttggcgctctatctagcgctctttctcgtctccaatcgaacccagcactcagagaactgagggaggtcgcaaagggaatgactaagcaaattggtggtcccatgcatcaattagctgctaaggtggatgagtatgcacgcggaatgatctcgggcacgggcagttctctgtttgaggagctggggctctattatattggaccagtggatggacacaacattgacgatctcgttgctatattaaaggaagttaagtctacgaggacaacaggtcctgttcttattcatgttgtaaccgaaaaggggcgtggatatccctatgcagaacgtgctgatgataagtatcatggagtcgtcaaatttgatccagcaactgggaggcagttcaaaaccaccaataaaactcagtcctacacgacgtattttgccgaagctctcgtggctgaagctgaggtagacaaagacgttgttgctatacatgcagctatgggaggaggaacaggtcttaacctgtttcaacgtagatttcctactcgatgtttcgatgtggggattgctgagcaacatgcggtcacgtttgccgctgggcttgcatgcgaaggtttaaaacctttctgtgcgatatactcttcttttatgcagagagcatacgatcaagtggtacatgatgttgatttgcaaaaacttccagttagattcgctatggatagggccggactcgttggagcagatgggccgactcactgcggcgcgtttgatgttacattcatggcatgtcttccaaatatgattgtgatggcaccaagcgatgaggcagatcttttcaatatggtggctacggccgttgcaatcgatgatagaccaagttgcttccgataccctcggggaaatggaataggcgttgcacttcctcctggaaacaagggagtccctattgaaataggtaaaggacgtatcttgaaagagggtgaaagggttgcacttctcggctacggttctgcagttcagtcttgtttgggagctgcggttatgctagaagaaagagggttaaatgtcacagttgcggacgccagattctgtaaaccgttggacagagcgcttattaggtcacttgctaagtctcacgaagttttgatcactgtggaagagggttccattggtggatttggttcgcacgtggtacagtttttggctttagatggtttgttggatggcaagcttaaatggcggcctatggtcctacctgataggtacattgatcatggtgctccggctgaccaacttgccgaggctggtcttatgccttcacacattgctgccaccgctttaaatttgatcggagctccacgagaagctctattctga

SEQ ID NO: 5 - DNA, 1224 bp, Hordeum vulgare
atgcaagccgtcacggcggcggccgcggcggggcagctgctaacagatacgaggagagggcccagatgtagggctcggctgggaacgacgagattatcctggacaggtcgatttgcagtggaagcttttgcaggccagtgccaaagtgctactactgtaatgcataaattcagtgccatttctcaagctgctaggcctagaagaaacacaaagagacagtgcagcgatgattatccagccctccaagctggatgcagcgaggttaattgggatcaaaacggttccaacgccaatcggcttgaggaaatcaggggagatgttttgaagaaattgcgctctttctatgaattttgcaggccacacacaatttttggcactataataggtataacttcagtgtctctcctgccaatgaagagcatagatgattttactgtcacggtactacgaggatatctcgaggctttgactgctgctttatgtatgaacatttatgtggtcgggctgaatcagctatatgacattcagattgacaagatcaacaagccaggtcttccattggcatctggggaattttcagtagcaactggagttttcttagtactcgcattcctgatcatgagctttagcataggaatacgttccggatcggcgccactgatgtgtgctttaattgtcagcttccttcttggaagtgcgtactccattgaggctccgttcctccggtggaaacggcacgcgctcctcgctgcatcatgtatcctatttgtgagggctatcttggtccagttggctttctttgcacatatgcagcaacatgttctgaaaaggccattggcagcaaccaaatcgctggtgtttgcaacattgtttatgtgttgcttctctgccgtcatagcactattcaaggatattccagatgttgatggagatcgagactttggtatccaatccttgagtgtgagattggggcctcaaagagtgtatcagctctgcataagcatattgttgacagcctatggcgctgccactctagtaggagcttcatccacaaacctatttcaaaagatcatcactgtgtctggtcatggcctgcttgctttgacactttggcagagagcgcagcactttgaggttgaaaaccaagcgcgtgtcacatcattttacatgttcatttggaagctattctatgcagagtatttccttataccatttgtgcagtga

SEQ ID NO: 6 - protein, 57 aa, Pisum sativum
MASMISSSAVTTVSRASRGQSAAVAPFGGLKSMTGFPVKKVNTDITSITSNGGRVKC

SEQ ID NO: 7 - protein, 410 aa, Zea mays
MAIILVRAASPGLSAADSISHQGTLQCSTLLKTKRPAARRWMPCSLLGLHPWEAGRPSPAVYSSLPVNPAGEAVVSSEQKVYDVVLKQAALLKRQLRTPVLDARPQDMDMPRNGLKEAYDRCGEICEEYAKTFYLGTMLMTEERRRAIWAIYVWCRRTDELVDGPNANYITPTALDRWEKRLEDLFTGRPYDMLDAALSDTISRFPIDIQPFRDMIEGMRSDLRKTRYNNFDELYMYCYYVAGTVGLMSVPVMGIATESKATTESVYSAALALGIANQLTNILRDVGEDARRGRIYLPQDELAQAGLSDEDIFKGVVTNRWRNFMKRQIKRARMFFEEAERGVTELSQASRWPVWASLLLYRQILDEIEANDYNNFTKRAYVGKGKKLLALPVAYGKSLLLPCSLRNGQT

SEQ ID NO: 8 - protein, 492 aa, Erwinia uredovora
MKPTTVIGAGFGGLALAIRLQAAGIPVLLLEQRDKPGGRAYVYEDQGFTFDAGPTVITDPSAIEELFALAGKQLKEYVELLPVTPFYRLCWESGKVFNYDNDQTRLEAQIQQFNPRDVEGYRQFLDYSRAVFKEGYLKLGTVPFLSFRDMLRAAPQLAKLQAWRSVYSKVASYIEDEHLRQAFSFHSLLVGGNPFATSSIYTLIHALEREWGVWFPRGGTGALVQGMIKLFQDLGGEVVLNARVSHMETTGNKIEAVHLEDGRRFLTQAVASNADVVHTYRDLLSQHPAAVKQSNKLQTKRMSNSLFVLYFGLNHHHDQLAHHTVCFGPRYRELIDEIFNHDGLAEDFSLYLHAPCVTDSSLAPEGCGSYYVLAPVPHLGTANLDWTVEGPKLRDRIFAYLEQHYMPGLRSQLVTHRMFTPFDFRDQLNAYHGSAFSVEPVLTQSAWFRPHNRDKTITNLYLVGAGTHPGAGIPGVIGSAKATAGLMLEDLI

SEQ ID NO: 9 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 10 - protein, 717 aa, Arabidopsis thaliana
MASSAFAFPSYIITKGGLSTDSCKSTSLSSSRSLVTDLPSPCLKPNNNSHSNRRAKVCASLAEKGEYYSNRPPTPLLDTINYPIHMKNLSVKELKQLSDELRSDVIFNVSKTGGHLGSSLGVVELTVALHYIFNTPQDKILWDVGHQSYPHKILTGRRGKMPTMRQTNGLSGFTKRGESEHDCFGTGHSSTTISAGLGMAVGRDLKGKNNNVVAVIGDGAMTAGQAYEAMNNAGYLDSDMIVILNDNKQVSLPTATLDGPSPPVGALSSALSRLQSNPALRELREVAKGMTKQIGGPMHQLAAKVDEYARGMISGTGSSLFEELGLYYIGPVDGHNIDDLVAILKEVKSTRTTGPVLIHVVTEKGRGYPYAERADDKYHGVVKFDPATGRQFKTTNKTQSYTTYFAEALVAEAEVDKDVVAIHAAMGGGTGLNLFQRRFPTRCFDVGIAEQHAVTFAAGLACEGLKPFCAIYSSFMQRAYDQVVHDVDLQKLPVRFAMDRAGLVGADGPTHCGAFDVTFMACLPNMIVMAPSDEADLFNMVATAVAIDDRPSCFRYPRGNGIGVALPPGNKGVPIEIGKGRILKEGERVALLGYGSAVQSCLGAAVMLEERGLNVTVADARFCKPLDRALIRSLAKSHEVLITVEEGSIGGFGSHVVQFLALDGLLDGKLKWRPMVLPDRYIDHGAPADQLAEAGLMPSHIAATALNLIGAPREALF

SEQ ID NO: 11 - protein, 407 aa, Hordeum vulgare
MQAVTAAAAAGQLLTDTRRGPRCRARLGTTRLSWTGRFAVEAFAGQCQSATTVMHKFSAISQAARPRRNTKRQCSDDYPALQAGCSEVNWDQNGSNANRLEEIRGDVLKKLRSFYEFCRPHTIFGTIIGITSVSLLPMKSIDDFTVTVLRGYLEALTAALCMNIYVVGLNQLYDIQIDKINKPGLPLASGEFSVATGVFLVLAFLIMSFSIGIRSGSAPLMCALIVSFLLGSAYSIEAPFLRWKRHALLAASCILFVRAILVQLAFFAHMQQHVLKRPLAATKSLVFATLFMCCFSAVIALFKDIPDVDGDRDFGIQSLSVRLGPQRVYQLCISILLTAYGAATLVGASSTNLFQKIITVSGHGLLALTLWQRAQHFEVENQARVTSFYMFIWKLFYAEYFLIPFVQ

SEQ ID NO: 12 - protein, 322 aa, Capsicum annuum
MASISSLNQIPCKTLQITSQYSKISSLPLTSPNFPSKTELHRSISIKEFTNPKPKFTAQATNYDKEDEWGPELEQINPGGVAVVEEEPPKEPSEMEKLKKQLTDSFYGTNRGLSASSETRAEIVELITQLESKNPTPAPTEALSLLNGKWILAYTSFSGLFPLLARGNLLPVRVEEISQTIDAETLTVQNSVVFAGPLSTTSISTNAKFEVRSPKRLQINFEEGIIGTPQLTDSIELPENVEFLGQKIDLSPFKGLITSVQDTATSVAKSISSQPPIKFPISNSYAQSWLLTTYLDAELRISRGDAGSIFVLIKEGSPLLKP

SEQ ID NO: 13 - protein, 307 aa, Brassica oleracea
MSSLGRILSVSYPPDPYTWRFSQYKLSSSLGRNRRLRWRFTALDPESSSLDSESSADKFASGFCIIEGPETVQDFAKMQLQEIQDNIRSRRNKIFLHMEEVRRLRIQQRIKNTELGIINEEQEHELPNFPSFIPFLPPLTAANLKVYYATCFSLIAGIILFGGLLAPTLELKLGIGGTSYADFIQSLHLPMQLSQVDPIVASFSGGAVGVISALMVVEVNNVKQQEHKRCKYCLGTGYLACARCSSTGALVLTEPVSAIAGGNHSLSPPKTERCSNCSGAGKVMCPTCLCTGMAMASEHDPRIDPFD

SEQ ID NO: 14 - protein, 426 aa, Zea mays
MMSTSRAVKSPACAARRRQWSADAPNRTATFLACRHGRRLGGGGGAPCSVRAEGSNTIGCLEAEAWGGAPALPGLRVAAPSPGDAFVVPSEQRVHEVVLRQAALAAAAPRTARIEPVPLDGGLKAAFHRCGEVCREYAKTFYLATQLMTPERRMAIWAIYVWCRRTDELVDGPNASHISALALDRWESRLEDIFAGRPYDMLDAALSDTVARFPVDIQPFRDMIEGMRMDLKKSRYRSFDELYLYCYYVAGTVGLMSVPVMGISPASRAATETVYKGALALGLANQLTNILRDVGEDARRGRIYLPQDELEMAGLSDADVLDGRVTDEWRGFMRGQIARARAFFRQAEEGATELNQESRWPVWSSLLLYRQILDEIEANDYDNFTRRAYVPKTKKLMALPKAYLRSLVVPSSSSQAESRRRYSTLT

SEQ ID NO: 15 - DNA, 860 bp, Sorghum bicolor
gaattctcaatagctatagttcaacaaatactccctccactctagtttattacatgctctaggttttcgctaactcccctaactttcacttacttcctactaaatctatctaaaattttgaacatcaagttttttcattgaattattaatgataatgtcatgatagcgcttattctcatcaaagtttatttaaaactttttgatagttatacaatgttgggtatagataattcaaaaataaattgtaacaaggaacacagggagtgtgtggcaactgtcatgtttggctataagcattctaaatttataaattctatgtgtacataatggtatttttatttagtctcaatctttagcatttgtttattcattgagtaacttctcgcctaactaccgtgctatcttcaaccatgagtacaatactacaagaacgtccgttgataaaggctttgatccacatgagcaagtcataactttacatactcgccatgtatataaagtgaacatttatgatgtggctaaggttgtaacatgtgtaaaggtgaagtgatcatgcatgttatttctattgtatcaaaaaaactccaatagaaaacaacaagtgtttcttgtacttagtggaaattgtctttcatacatagaccatataatccaacaaaaataataactaaatgtcaaaattgactaggtgccatgtcatctatagcttatctgttgtttgaaaaaaggcaaaatctaaacaggagccctcacttgtataaatatataggccccagatcagtagttaatccatcgcccataacactgagagcaatctgaaacataccaagggaaacaaacgtcaacgtccttcaccaacc

SEQ ID NO: 16 - DNA, 494 bp, Sorghum bicolor
gcacctgagagtgatctacctgaataagtactcgtggactgtaataaacaaagcttgttcatgggtaaactgcatgtctgctgcatggatgagtctttcaactacatatatagctcgtcaaatagaacaacttaacttaagtgagtaatgtttcaaatgagaacttgtgtcagggaaaaaatgagaacttgtgtcagggaaaccaattccaagttccaacttatctacatagatgtggcaattagtcactctgtcacatggggaaccaaatattcaatagcagataacagagtacaaatatatcgattcaccatctgaaccaactactacctacggttaaagcttgaaattacccactggtgcattgatttatagtttgcagaaactaaaaagtataagaccacaccacatctatctacatgtccaactccaacctaaaggtcaatctccatctggcgtttcctcatcatcagtgttgtcgcctatctaagctt

SEQ ID NO: 17 - DNA, 171 bp, Pisum sativum
atggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgc

SEQ ID NO: 18 - DNA, 833 bp, Sorghum bicolor
gttaccgaattcggcctgtttggtttatgcccaaatttgccatacctaacttttggcaaactgtagcaaagtttagcaagaacatgagtctatgatgtgagggacggtgctaagaaaatacggtgtgtcatagttacagcaacgaactaaacacacttgacttgtgccatggcaagttgtgatcacgaaccaaataggccttaccccttacgtgcaaaccgataaagtcatgtggagtggcaatcattccaaggtattctactaacaaccagcacaacattacaaacttggtttcaccaaggcctgaactcacagtacactaccttcacatgtagagtgaatgggtgatgagtcatgcatgctgatttgtcaaggtgtgcatgaactgatggtgatgagtcatgctgatgtgtgaagcaatactgctcagcgtagcccaatttatctcaacaaaaaaacaaacaaacacacacgtatgccattacaaagttagcttcacaagcgtatgaataattcagtgacaatccttgacatgtaaagttgattttcatatgtgctgacaggaagctcaatgatctatttatacatccaaatccatgtaaaaaggcacttgtatttccacgtcatgcaatgcaacgacattccaaaaatcatcagttgcagatgctgcagaatgcagcaaaccatggatcatctataaatagctcccacatatgcactactactctatcatcagatcccacatcaagatcagagacactactactgcaccaaactaattaagcaagcaaagcagagccgtagagaggagcgctcaacagatcaggtgtagc

SEQ ID NO: 19 - DNA, 462 bp, Sorghum bicolor
actaactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgctt

SEQ ID NO: 20 - DNA, 896 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 21 - DNA, 82 bp, Zea mays
tccccaacctcgtgttgttcggagcgcacacacacacaaccagatctcccccaaatccacccgtcggcacctccgcttcaag

SEQ ID NO: 22 - DNA, 1013 bp, Zea mays
gtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 23 - DNA, 311 bp, Solanum tuberosum
cctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 24 - DNA, 1510 bp, Zea mays
ttatataatttataagctgaaacaacccggccctaaagcactatcgtatcacctatctgaaataagtcacgggtttcgaacgtccacttgcgtcgcacggaattgcatgtttcttgttggaagcatattcacgcaatctccacacataaaggtttatgtataaacttacatttagctcagtttaattacagtcttatttggatgcatatgtatggttctcaatccatataagttagagtaaaaaataagtttaaattttatcttaattcactccaacatatatggattgagtacaatactcatgtgcatccaaacaaactacttatattgaggtgaatttggatagaaattaaactaacttacacactaagccaatctttactatattaaagcaccagtttcaacgatcgtcccgcgtcaatattattaaaaaactcctacatttctttataatcaacccgcactcttataatctcttctctactactataataagagagtttatgtacaaaataaggtgaaattatgtataagtgttctggatattggttgttggctccatattcacacaacctaatcaatagaaaacatatgttttattaaaacaaaatttatcatatatcatatatatatatatacatatatatatataaaccgtagcaatgcacgggcatataactagtgcaacttaatacatgtgtgtattaagatgaataagagggtatccaaataaaaaacttgttcgcttacgtctggatcgaaaggggttggaaacgattaaatctcttcctagtcaaaattgaatagaaggagatttaatctctcccaatccccttcgatcatccaggtgcaaccgtataagtcctaaagtggtgaggaacacgaaacaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgaca

SEQ ID NO: 25 - DNA, 473 bp, Zea mays
agaaactatgtgctgtagtatagccgctgcccgctggctagctagctagttgagtcatttagcggcgatgattgagtaataatgtgtcacgcatcaccatgcatgggtggcagtgtcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgagtcgac

SEQ ID NO: 26 - DNA, 1294 bp, Sorghum bicolor
tatcaaggttgcttcaaacactgcacgatatataacacacaatagttgagggagttaattaagtagaatttttcataaatgtgaaaacacagatgcaatatcattccatcaatgaaggccatcgacaccagctcaagctaccattggtgatgtagtagtgatcatcatcgggtatgaattgaacagatgtcgatcaattgatcatcactgccgacagtgaccactgacctgtggtgatcaacgcttgtcatatatagactgcagcactgatcagtggtgtaggaactctcaacattgccggtgtgtacaactgaccgatggtgaccaaatgaccatcattgttggattttgttataaacccgacaacatttgtaggatcttcaacgcctgaacaaaaaataaactcagcgatgatattgatgttatcgcagctagattgaactccaaaatggtggtgatagcatataaccaccactaattatgaactggacatgattaccatggccatcactgtcgataattcactgccgagtgctggaagtggcggcgatgcaggtttccaaactggttgtgctgatctttctgtaatagtgttagttgaaaatgacttaggtatttgtcaatctttaggtattgactataatcaagtggaatagatattatggtgttgtttggttagtggagtgtcaataaaagtgaaatggaaatgaatattgttgttccccaaatactttgattgtttggttcttgtgaggtaacactgccgttgacatgatccatttgtggtaggttatgggtggtaaccgcattgtacgacatggaacgagtgactccttgaaccgattatagcacaaaataaccaaatgcaaattaacattggtgaataccaccatgaattttttttctggtgcaaaatagcctaaccaagccgcacatatgtggctaaggctacacatgtgtaaaggtgaatggatcgagccattgtcacccatgtatttggaaaataccaagaggacaaaaccacttatttagtgtattttgtggagattgtattgcagatgtataaagtataacccaacaaagtggcaactaaatgtcaaaaccaactagataccacgtcatctctagcttatcttactattatgttttttggtaaaagccaaaataaatcttgcacaaccacaaggcttaacatgaatataaataccccccagatcagtaggtaatccatcacccatattattgagaccaactagcaacatagaaagtggaatacactagcaacatagcaacc

SEQ ID NO: 27 - DNA, 499 bp, Zea mays
atctgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttaggcaagggagagagagagcg

SEQ ID NO: 28 - DNA, 1441 bp, Sorghum bicolor
gaattcgagagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatcccgcgccacctccgctttagttctgcatacagccaacccaaggatccaacacacacccgaggatatcacagt

SEQ ID NO: 29 - DNA, 1000 bp, Sorghum bicolor
gctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaagaattc

SEQ ID NO: 30 - DNA, 344 bp, Zea mays
gtacagtacacacacatatgtatatatgtatgatgtatcccttcgatcgaaggcatgccttggtcgaataactgagtagtcattttattacgttattttgacaagtcagtagttcatccatttgtcccattttttcagctaggaagtttggttacactggccttggtctaataactgagtagtcattttattacgttgtttcgacaagtcagtagctcatccatctgtcccatttttttcagctaggaagtttggttacactggacttggtctaataactgagtagtcattttattacgttgtttcgacaagtcattagctcatccatctgtcccatttttcag

SEQ ID NO: 31 - DNA, 2154 bp, Arabidopsis thaliana
atggcttcttccgccttcgctttcccgagctacatcatcacgaagggtggactgtcaaccgatagctgcaagagcacgtctctttcgtcgtcacggtccttggttactgatctgccgagcccgtgcctgaaaccgaacaacaactcacacagcaatcgccgcgccaaggtgtgtgcttccctggctgagaagggcgaatactactcgaacagaccgcccacgcctttgctcgataccatcaactaccccatccacatgaagaacctgtccgtgaaggagctcaagcaactgtccgacgagctccggagcgatgttatcttcaacgtgtcgaaaacaggcggacacctcggttcgtcacttggagttgtggagctgacggtcgcgcttcactacatcttcaacaccccgcaggacaagatcctgtgggatgtcggccaccagtcatatccgcacaagatcctcaccggtcgcagaggcaagatgcccaccatgagacagacgaacggcctgtcaggtttcaccaagcgcggcgagagcgagcacgactgctttggaaccggccactcctcaaccacgatctccgctggacttggcatggcagttggccgcgacctcaagggtaagaacaacaacgtcgtcgcggtcattggagatggcgctatgaccgccggtcaagcgtacgaggccatgaacaacgccggctacctcgactcggacatgatcgtcatcctgaacgacaacaagcaggtgtcattgccgactgcgaccctggatggtccttcaccgccagttggagcactctcctcagcgctcagcagactgcagtcgaacccagccttgcgggagctgagagaagtcgctaagggcatgaccaaacagattgggggtccgatgcaccagcttgcggctaaagttgacgaatacgcacgcgggatgatttccggcaccggctcatcgttgttcgaggagctcgggctgtactacatcggcccggttgacggccacaacatcgatgacctggtggcgatcctcaaggaggtgaagtcgacgaggaccaccggacccgtccttatccacgtggtgacggagaagggtcgcggctacccgtatgcggagagagccgatgacaagtaccacggcgtcgtgaagttcgacccagctactggccgccagttcaagacgaccaacaagacgcagtcctacaccacgtacttcgctgaggcactcgtcgctgaagccgaagtcgacaaggacgtggtggcaattcacgctgcaatgggcggcggtacgggtctgaacctgttccagcggagatttcccactaggtgcttcgatgtcggaatcgccgagcagcacgccgttacatttgccgccggacttgcgtgtgaaggactgaaacctttctgcgccatctactcgtcgttcatgcagcgcgcatacgaccaggtcgtccacgatgttgaccttcagaagttgcccgtccgcttcgcgatggacagagctggactcgtcggcgctgatggaccaactcactgcggcgctttcgatgttaccttcatggcctgcttgccgaacatgatcgtgatggcgccctccgacgaagctgacctgttcaacatggttgctactgcagtggcgatcgacgatcgcccatcgtgcttcagatacccgcgcggaaacggtattggtgttgccctgccgccgggtaacaagggcgttccaatcgagatcgggaagggcaggatcttgaaggagggtgagcgcgtggcgttgctcggttatggctcggctgtccagagctgcctcggagctgcagtgatgctggaggaacgcggcctgaatgttacagttgccgatgcgcgcttctgcaaaccgctggacagggctctcattcggtccctcgccaagtcccacgaggttctgatcaccgtcgaagaaggtagcatcggcggcttcggatcgcacgtcgttcagttccttgctttggacgggctgcttgacggcaagctcaagtggaggccgatggtccttcccgacagatacatcgaccacggcgcacctgccgatcagctggcggaagctggactgatgccgtcacacatagcggctacagccctgaacctcatcggcgctcctagagaggcgctcttctag

SEQ ID NO: 32 - DNA, 818 bp, Sorghum bicolor
tagctagcttttctaaatatattaatttttgttatgcatgcatctagacatgcatggcgcataataactaagtgcattgcaaaaactataaatttagaaaaaccgaaatattttataatatagaattgagggagtattagttaggctatgcctccttatcatttcgttgatgatctagagtactctagctatccccaagagtaggccggatggcggcacggccacgaaatttgtaggtgaaaacatgtagcagtgttagagaagagtaggcagatcgcacaatgcaaatgcacctggacagtcgtacgtgcgtgtatatatgtaaactaaaggcgcaacaaactgttggagtcagtacaaaactgaatcggcctttctgactgtcagcacaagcaacaagtcgaagcgatcgatcatccacgtcgatctctaatgctggttaatcaagtttgttagctagatacaaatgtattatttggcatatatgtgtaaaaatgcatgtaacaccagcgagttacatgtctaacttgtcatattcccaaccaacactcttatcacagcaaagcaagcactagctagcatacaaaagacaaggcctgaatttgttcagaggtgccacacttttttcttgcatcttttcatttcatatcattccttttagtttattcccatttatttttatttttctggaacaccagcagcacattcctttgctatatataaaaaaaaaagaccccggacgggcctctgctagctagcactgcacacacggccggcaacagcactctgtcagtgaagagagtgagtgagcagaagcc

SEQ ID NO: 33 - DNA, 1233 bp, Zea mays
atggccatcatcctggtgagagccgcatcgcccgggcttagcgctgctgactctataagccaccaaggcaccctccagtgctccacgctgctcaaaaccaagaggccagctgcaagacgctggatgccgtgcagcttgttgggcctgcacccttgggaagctggtagaccatccccggcggtgtactcgtcgctgccggtcaacccagcgggtgaggctgttgtgtcgtccgagcagaaggtctatgacgtggtcctcaagcaggccgccctcctcaagagacaactgcggactccagtgttggacgctcggcctcaggacatggatatgcctcgcaacggacttaaggaggcatacgaccggtgtggagaaatctgcgaggagtacgcgaaaaccttctacctcggcacgatgctgatgactgaagaaagacgcagggccatctgggctatctacgtgtggtgcagaaggactgacgagctggtcgatggaccgaacgctaactacatcacgcccaccgccctggacagatgggagaagagactggaggacctgttcaccggtcgcccatatgacatgctggatgctgccctctcggacactattagccggttcccgatcgacatccaacctttccgcgacatgatcgagggaatgcgctcagacctgcgcaagacccggtacaacaacttcgacgagctgtacatgtactgctactacgtcgccggcaccgttggactcatgtcagttccggtgatgggcatcgccacagagagcaaggctacaacggagtctgtttactccgccgcgcttgcactcggcattgccaaccagctgacaaacattctcagggacgtcggagaggatgcgcgcagaggtcggatttatctcccacaggacgaactggcccaagccgggctgtcggatgaggacatcttcaagggcgtcgtcaccaacaggtggcgcaacttcatgaagaggcagatcaagcgcgctaggatgttctttgaggaggccgagagaggagtgaccgagctgtcgcaagcgtcaagatggcccgtgtgggcctcgctgcttttgtatcgccagatcctggacgagatagaggcgaacgactacaacaacttcacgaagcgcgcctacgtcggtaagggcaagaaacttctggcgctgcccgtggcctacggaaagtcactcctcctcccatgctccctgcggaacggacagacgtag

SEQ ID NO: 34 - DNA, 261 bp, cauliflower mosaic virus
tagtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctc

SEQ ID NO: 35 - DNA, 1233 bp, Zea mays
atggcgattatcctggtgagggcagcgtcacccggactgagcgcggcagattccatctcccaccaggggactctgcaatgttccaccctcttgaaaacgaagaggccggctgcccgcagatggatgccctgctctcttttgggattgcacccttgggaggccggcagaccgtcgccagctgtctactcctcgctcccggtgaacccagccggtgaggcagtggtgtcgtcggagcaaaaagtctatgatgtcgtgctgaagcaggctgccctcctcaagaggcagctccgcacgcctgtcctcgacgccagaccacaggatatggacatgcccagaaacggcttgaaggaggcgtacgatcgctgcggcgagatatgcgaagagtacgccaagaccttctacctcggcaccatgctcatgacagaagagcgccgcagggccatttgggcaatatatgtgtggtgccggagaaccgacgagctggtcgatgggccgaacgcaaactacatcacgccaacagcgcttgataggtgggaaaagaggcttgaggaccttttcaccggcagaccctatgacatgctcgacgccgcgctgtccgacaccattagccgcttccccatcgacatccagccgttccgggacatgatcgaagggatgaggagcgacctgcgcaagacccgctacaacaatttcgacgagctctacatgtattgctactacgtggcgggaaccgtcggtttgatgagcgtcccggttatgggcatcgcaacagagtccaaggcgacgacagaatcagtctactcggcagcattggcactcgggatcgcgaaccagcttacaaacatcctgagggacgttggcgaggacgccagaagaggcagaatctacctgccgcaggatgaactggcgcaagctgggctgtcagacgaggatattttcaagggtgtcgtgactaacaggtggcggaacttcatgaagcgccaaatcaagcgcgctcgcatgttcttcgaggaggcggaaagaggtgtcacagaattgtcgcaggccagcaggtggcctgtttgggcctccctcctgttgtatcggcagatactcgacgagatcgaggccaacgactacaataacttcaccaagcgggcctacgtgggcaagggcaagaaactgctggccctgccggtggcttacgggaagtcactgcttctcccgtgctcgctccgcaacggacagacctag

SEQ ID NO: 36 - DNA, 1784 bp, Capsicum annum
atggcgtccatcagctccctcaatcaaatcccgtgcaagaccctccagatcacgtcccagtactcgaagatctccagcttgccgctcacgagcccgaatttcccgagcaagacggagctgcaccggtccatctcaatcaaggagttcacgaacccgaaaccaaaattcaccgcgcaggccactaactacgacaaggaggacgaatggggcccggagctcgagcaaataaatccgggcggcgtggccgtggtggaggaagagccacctaaggagccctccgagatggagaagttgaagaagcagctcaccgacagcttctacggcactaaccgcggacttagcgcttcctccgaaacgagggccgaaattgtggaactgatcacccagttggagtccaagaatccgacacctgcaccaaccgaggcgctctcccttctcaacggcaaatggatactggcgtaagttctttttttttttttagctgcaccatgaaaaatatatgctttattacacggtccatattactggtctgtcggagaaatccaattttttcctctacagaaatgctaagataagataaacccttttgatttggtcctcttggacctgcatattgctttagtgtaacagtttttttttaaagtagagtagtactattcagaggtggatcaagatttggaggttatgagttcgcataatgatttcaagttaatatgcaatagtcgctaggttcacagctaaatacaacaacataacaattgtaatccgacaagtggggtctggagagttcagagtgtaggtagaccttagccctgctttaggtaaatagagtctgttcccatagaccctcggctcatgcaaaaaaaatatcaaagaagatattatataaagcatgacaaaactactaccacagatgatataaatatttagggatgattaatagattctgaaagaacttcttttcttaatgtttgatgccctttttttaccccttggtaagctggattctgttaatctttaacggagtattgcagtttgatgtggagaaaagccttctttgagcatcaatttcttagtcatgagaatgcaggtggcatcttttcaccaccataattggtcccttgttgtactctagctcatcattatcttttgatgaagacaatgacattgtttagtccccgaagaacgttaaatgttcttgcttcagtgtatatgtgattactcgcgcttgttggcatacgaacacttggaattctgtactgaaacactgcaggtacacgtcctttagcggcttgttcccgctgctcgcgcgcggaaacctcttgcctgtgagggtggaggagatctcgcaaacaatagacgcggagacgctcaccgtgcagaattctgtggtgttcgccggaccactgagcacgacgagcatcagcaccaacgcgaagttcgaggtgcggtcgccgaagaggctccagatcaacttcgaggaggggattataggcacgccgcagctcaccgactcgattgagctgccggaaaatgtggagttcctcggccagaagatcgacctttcgcccttcaagggcctgatcacgtccgtgcaggacacggcgacttccgtggcgaagtcgatctccagccagccaccaatcaagttcccgatctcgaacagctacgcccagtcctggctgctcactacgtacctggacgccgaactgaggatctcacgcggagacgccggctcgatcttcgtgctgatcaaggagggctcgccactcctgaagccgtag

SEQ ID NO: 37 - DNA, 532 bp, Sorghum bicolor
gaattcttttcaagggattgggtcagaaacaaatcgtctccgtgtacaacgaagtggtgagtcatgagccatgttgatctgatatatacatagcacacacgacatcacaaacaagtcatactacattacagagttagtttcaactttcaagtaaaaacaaagtaggccggagagaggacaataatccttgacgtgtaaagtgaatttacaaagccatatatcaatttatatctaattcgtttcatgtagatatcaacaacctgtaaaaggcaacaaattgagccacgcaaaattacaagtgagtccaaataaaccctcacatgctacataaaagtgaatgatgagtcatgtatatctggcaagaaactgtagaagctacagtcatcggtagcaaagaaacacaagaaaatgtgctaataaaagctataaataaccctcgtacgcctatgcacatctccatcaccaccactggtcttcattcagcctattaacttatatctatctactccagagcagacaagagctcgacacc

SEQ ID NO: 38 - DNA, 924 bp, Arabidopsis thaliana
atgagcagcttgggccgcatactgtcagtctcctaccctcccgacccatacacctggcggttctcccagtacaagctctcatcctccctgggtagaaaccgcaggctccgctggagattcacggccctcgatcccgaatcgtcatcactggacagcgagtcgtcagcggacaagtttgcctccggcttctgcatcatcgagggcccggagacggtgcaagatttcgcgaagatgcagctgcaggagatccaggacaatatccgcagccgccggaacaagatcttcctgcacatggaggaggtccgccgccttagaatacaacagcggattaaaaacaccgagctcgggattattaacgaggagcaagagcacgagctcccgaacttcccgtcgtttatcccgttcctcccgccacttaccgccgccaacctcaaagtctactacgcgacgtgcttctcgctgatcgccgggatcattctcttcggaggactcctggcacctactcttgagttgaagctcggcatcggcggaacctcctacgccgacttcatccagtcactccacctgccgatgcagctctcccaggttgacccaatcgtggcgagcttctcaggaggcgccgttggagttatctcggctcttatggtggtcgaggtgaacaacgtgaagcagcaggagcacaagcgctgcaagtactgcctcgggacgggttacctcgcgtgcgcaagatgctcatcgactggagcccttgtcttgaccgagccggttagcgctatcgccggtggcaaccactcattgagcccacccaagacagagaggtgctcaaactgctccggcgctggcaaggtgatgtgcccaacgtgcctgtgcaccgggatggcgatggcatcggagcacgatcccaggatcgatccgttcgactag

SEQ ID NO: 39 - DNA, 878 bp, Zea mays
tcacttaagatgtactcgacaatggtgccctcataccggcatgtgtttcctagaaataatcaatatattgattgagatttatctcgatatatttctgaactatgttcatcatataaataactgaaaacatcaaatcgtaattttaaactcatgcttggtcaatacatagataatacaatattacttcatcatcccaatgatgtcctagcacaacctattgaatgttaatgtttggttgtgtgggggtgtgtttataacatagatgtgattatttgtgctttttgttgagtatatacatatatggtatgttgatttgatatagtgatggacacatgctttggccttggatattcaaatcacttgtacttgcacgaagcaaaacataatataagtttagaagtaaacttgtaactgtgtccaaacatgctcacacaaagtcatatcgcattatatttttttggtaaatattcaacacatgtattttttacaagaacccaaattttacagacaaatgcagcattgtagacatgtagaattctttgaagcatgtgaacttaacaacactaatgtcattaaatcaactggaccctatgagtaacaatttcgatattgcaaacaccaaattatggaacttatttgctgaaaaaattatgatcaatgtgaagtttaaattattataccataaatatatcaaagattttttttgaggaaggtaaaaattgcatggaatgggctgcccaacgtgatagctcacttttatgctaggtagcattaccaaagatgggaatgttctgatgaacaccaaacccactcaaataatatttatatttgggttgtttagttgtaaaagtgaagacccaagattaaagtaccaattg

SEQ ID NO: 40 - DNA, 1709 bp, Zea mays
gaccgttgccttctgcccgttggcacaccggacagtccggtgcacaccggacagtccggtgctacagccagagagcgcctgtctgcggcctctctgcgccgactgtccggtgcacaccggacaccgatgtccggtgcgccaccaggcgctggctgacagcccttgtcttggatttcttcgctgatttcttcgggcttctttgttcttgagtattggactcctatgcatctttttatgtcttcttttgaggtgttgcatcctcattgccttggtccaattctcttcgcatcctgtgaactacaaacacaaacactagaagacttattagttcactgattgtgttgttcatcaaacaccaaaactcaattagccaaatggcccggggtccattttccttacaacttcaacggccgcaccgaccctctgacctctccttttctctcctttctcactcctatcggtagctacaacagaagcgacccccaacgcggcgcaaaccctcgaagcatacggctggggaagacggcagccaggtttatatcctaggcgcccgaggaaatcgcgcggtcagctgttacggttcgcccgcggggcacgattcgcgcgaagaagaccgtatgcaagggagggcccactagcagcgagccatcacctagggaagcgtgcatgcatcgattgacacgcgaccccaacagtcaggcgacccgagtgtgcagacggtcgtgatggtgaaagtggccggcccgcgcggacgcgtaggggcattgggccaaaatgcgtttcagcggcccagcttcttttttcttctatttttttctttccttttcctttctatttttagatttcaaatttaagttcaaattttttttatggtgaattttctaaaaatccgcagactagtatgaaaagaatttatatataaatctatttatttatatatttattttctatgttatttccaatttctaaaatgtaaattaggttaaatcgccatttggacactaatatatctttattagtattactattattagatgcacaaccaaataaactccaacatgatgcatcgattatttgtatgccattggttaattattcactttaaatatgctccttaacgattctcatgaaacagaaggccatgcacataaagatgtatccctttcttttatattcccagagttgggtattacaacattcatctatgcattctaggatttcaattaatctcaatcttttagtatttgttccttcattctcaaatcacttctcatctaactactatgcttgtttaaccagcacaacaatactacaacaatatccatttataaaggctttaatagcaaactttacatattcatatcatgttaaggttgtcacatgtgtaaaggtgaagagatcatgcgtgtcattccacataatgaaaagaattcctatataaaaacgacatgttttgttgtaggtagtggaaactatctttccagcaaagaccatataatccgataaagctgataactaaatgtcgaaatcgagtaggtgccatatcatctatatcttatctgttgtttggaaaaagacaaaatccaaaaaaaatatatgagatctcacctgtataaatagctcccaaatcagtagttaatacatctcccataatattttcagcattcagaaacacaccaagcgaagcgctctagcaacgacctaacaac

SEQ ID NO: 41 - DNA, 1281 bp, Zea mays
atgatgtctacgagccgcgcggtgaagtcgccggcgtgcgcggctcggcggcggcagtggtccgcggacgcgccgaaccgcacggcgacgttcctggcctgcaggcacgggaggcggctcggcggcggcggcggggcgccgtgctccgtgcgcgccgagggctccaacaccattggctgcctcgaggccgaggcgtggggcggcgcgccggcgctgccgggcctccgcgtggcggcgccgtcgcccggggacgcgttcgtcgtgccgtccgagcagagggtgcacgaggtggtgctcaggcaggcggcgctcgcggccgcggcgccgaggacggcgcggatcgagccggtgcccctggacggcgggctgaaggcggccttccaccgctgcggcgaggtctgcagggagtacgccaagacattctaccttgcgacgcagctgatgacgcccgagaggaggatggcgatctgggcaatatacgtgtggtgcaggagaacggacgagctcgtggacggccccaacgcgtcccacatctcggcgctggcgctggaccggtgggagtcgcggctggaggacatcttcgccggccggccgtacgacatgctcgacgccgccctgtccgacaccgtcgccaggttccccgtcgacatccagccgttcagggacatgatcgaggggatgcgcatggacctgaagaagtcccggtacaggagcttcgacgagctgtacctctactgctactacgtggccggcaccgtggggctgatgagcgtcccggtgatgggcatctcgccggcgtccagggcggccaccgagacggtgtacaagggggcgctggcgctgggcctggcgaaccagctcaccaacatcctcagggacgtcggcgaggacgccaggaggggacggatctacctcccgcaggacgagctggagatggcggggctctccgacgccgacgtcctggacggccgcgtcaccgacgagtggaggggcttcatgaggggccagatcgcgagggccagagccttcttcaggcaggcggaggaaggcgccaccgagctcaaccaggagagccgatggccggtgtggtcttctctgctcctgtaccggcagatcctcgacgagatcgaggccaacgactacgacaacttcacccggagggcctacgttccgaagacgaagaagctgatggcgctgcccaaggcgtacctgagatcactggtggtgccctcctcctcttctcaggctgagagccggagacgctattccaccctaacatag

SEQ ID NO: 42 - DNA, 517 bp, Sorghum bicolor
gtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctag

SEQ ID NO: 43 - DNA, 584 bp, Sorghum bicolor
gccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggcc

SEQ ID NO: 44 - DNA, 955 bp, Zea mays
gatccgattgactatctcattcctcaaaccaaacacctcaaatatatctgctatcgggattggcattcctgtatccctacgcccgtgtaccccctgtttagagaacctccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctacc

SEQ ID NO: 45 - DNA, 766 bp, Zea mays
taataaaagggtagtgtacgcctaccgcgtacgtacgtgtcaccgggcgtggcactctccagtctccagggacccatccaccaaatgctactgctccttcgtagggagacgtgggaataaagagtggtagctgcatgcacgtacggcggccatggctctccgatgagagagctagctgtgtacgtgtgttcttgatgttgttccatgcatgacatgtatacgtcttgcctaagtacgcttgtactagttgagagactgtgtaagtgaaatgtgctataataataaataagtaaaaggcgccttctccaacattctatgggctcgtttgggagggttgcggctcctctcaaaacggctccagctccaactcctccaaaggagtagctcctctgcaggagcccatgctttttgcaaatcgtttgctaaaaaacggctccttgtgtatgccgtgctcatcatcatcaacgttgattacctagaaaccactgcagtgctttctgttgtcggatagtggaaggctccttgtgtattattacaacaaaaaaatattatggagtaatattagaaaagcatttgcacatcacaatccatacacaagtcatatatcacttggatgattacctagaaagaaagatcgctcttgcgcgatgtcatcacgaaacctatccatcatacgatcattagagtatggacatgatgagtcagttgtatttctatatctaaaaggtatagtgggcacgtaatctggatttcgatcgcacttata

SEQ ID NO: 46 - DNA, 500 bp, Zea mays
aggcagcgcagagaggcagaggaggagctcgggtgtctgagggtcactccttacagtgatgctgggatcctcagccttgcaacattgtcttggcttagtccgttgctctccgttggtgcgcagcggccacttgagttggctgacatacccttgctggcgcacaaggaccgtgcaaagtcatgctataaggtgatgagcgctcactacgagcgccagcggctagaacaccctggtagggagccatcactgacatgggcaatactcaagtcgttctggcgagaggctgcggtcaatggtacttttgctgctgtcaacacaatcgtgtcatatgttggcccttacttgatcagctattttgtggactatctcagtggcaacattgctttcccccatgaaggttacatccttgcctccatattttttgtagcaaaattgcttgagacactcactgcccgacagtggtacttgggtgtggacatcatgggaatccatgtcaagtc

SEQ ID NO: 47 - protein, 309 aa, Erwinia uredovora
MNNPSLLNHAVETMAVGSKSFATASKLFDAKTRRSVLMLYAWCRHCDDVIDDQTLGFQARQPALQTPEQRLMQLEMKTRQAYAGSQMHEPAFAAFQEVAMAHDIAPAYAFDHLEGFAMDVREAQYSQLDDTLRYCYHVAGVVGLMMAQIMGVRDNATLDRACDLGLAFQLTNIARDIVDDAHAGRCYLPASWLEHEGLNKENYAAPENRQALSRIARRLVQEAEPYYLSATAGLAGLPLRSAWAIATAKQVYRKIGVKVEQAGQQAWDQRQSTTTPEKLTLLLAASGQALTSRMRAHPPRPAHLWQRPL

SEQ ID NO: 48 - DNA, 930 bp, Erwinia uredovora
atgaataatccgtccctcctcaaccacgccgtggagactatggctgtcggcagcaagtcttttgcaacggcctccaagttgttcgacgccaagaccagacgcagcgtgctgatgctctacgcctggtgcaggcattgcgacgacgtcatcgacgaccagaccttgggctttcaagccaggcaacctgccctgcagacgccagagcagagactcatgcagctcgagatgaaaacccgccaggcgtacgcgggctcacaaatgcatgagccggcgttcgcggctttccaagaggtcgcgatggctcacgacatcgctcccgcgtacgcttttgaccacttggagggcttcgcaatggacgtgagggaggcccaatacagccagcttgacgacacgctgagatactgctaccacgtcgctggcgtcgtgggccttatgatggcccagatcatgggcgtgagggacaacgctacgcttgatcgggcttgtgaccttggcctcgccttccaactgaccaacatcgccagggacatcgtggacgacgctcatgctggtaggtgttacttgcccgcgtcgtggctcgaacacgaaggcctcaacaaggagaactacgcggctccagagaatcgccaggccctctccaggattgcaagaaggctcgtgcaagaggcggaaccatactacctgtcggccacagcaggattggctggcctccctttgaggtcagcatgggctattgccaccgccaagcaggtctaccggaaaataggcgtgaaggtcgagcaagctggacagcaagcctgggatcaaaggcagtccacgaccaccccggagaaattaacgctcctcctggctgcttccggtcaggcattgacgtcaaggatgcgcgcacatccgcctagaccggcgcatctttggcaaaggccgctgtag

SEQ ID NO: 49 - protein, 35 aa, Coriandrum sativum
MAMKLNALMTLQCPKRNMFTRIAPPQAGRVRSKVS

SEQ ID NO: 50 - DNA, 105 bp, Coriandrum sativum
atggcgatgaaactcaacgccctgatgactctgcaatgccccaagaggaatatgttcacccggattgctccaccacaagctggtagggttaggtctaaggtgtcc

(19) United States
(12) Patent Application Publication
(10) Pub. No.: US 2014/0123339 A1
(43) Pub. Date: May 1, 2014

(54) Transformed Plants Having Increased Beta-Carotene Levels, Increased Half-Life and Bioavailability and Methods of Producing Such

(71) Applicant: PIONEER HI BRED INTERNATIONAL INC, JOHNSTON, IA (US)

(72) Inventors: MARC C. ALBERTSEN, GRIMES, IA (US); PAUL C. ANDERSON, ST. LOUIS, MO (US); PING CHE, AMES, IA (US); KIMBERLY F. GLASSMAN, ANKENY, IA (US); RUDOLF JUNG, LOHR AM MAIN (DE); ZUO-YU ZHAO, JOHNSTON, IA (US)

(73) Assignee: PIONEER HI BRED INTERNATIONAL INC, JOHNSTON, IA (US)

(21) Appl. No.: 13/788,128

(22) Filed: Mar. 7, 2013

Related U.S. Application Data
(60) Provisional application No. 61/720,455, filed on Oct. 31, 2012.

(51) Int. Cl. C12N 15/82

(52) U.S. Cl. CPC C12N 15/8243
USPC 800/278; 536/23.2; 435/320.1; 435/468; 800/298; 800/320

(57) ABSTRACT

Compositions and methods for increasing carotenoid levels and carotenoid half-life in plants are provided. The methods involve transforming organisms with nucleic acid sequences encoding enzymes associated with carotenoid biosynthesis and tocopherol and tocotrienols. In particular, the nucleic acid sequences are useful for preparing plants and microorganisms that possess increased beta-carotene levels and half-life. Thus, transformed bacteria, plants, plant cells, plant tissues and seeds are provided. The sequences find use in the construction of expression vectors for subsequent transformation into organisms of interest including plants, particularly sorghum.

### REFERENCE TO SEQUENCE LISTING SUBMITTED ELECTRONICALLY

The official copy of the sequence listing is submitted electronically via EFS-Web as an ASCII formatted sequence listing with a file named “5291_sequence_listing.txt” created on Mar. 6, 2013, and having a size of 83.2 kilobytes and is filed concurrently with the specification. The sequence listing contained in this ASCII formatted document is part of the specification and is herein incorporated by reference in its entirety.

### FIELD OF THE INVENTION

The present disclosure relates to the field of transformation of plant cells, seeds, tissues and whole plants. More specifically, the present disclosure relates to the insertion of recombinant nucleotide sequences encoding one or more of the enzymes specific of the carotenoid biosynthetic pathway into plant material in order to improve its agronomic and nutritional value. The present disclosure also relates to the insertion of recombinant nucleotide sequences encoding one or more of the enzymes specific of the vitamin E biosynthetic pathway into plant material in order to improve carotenoid half-life, bioaccessibility and bioavailability in plants.

### BACKGROUND OF THE INVENTION

Provitamin A (β-carotene) deficiency represents a very serious health problem leading to severe clinical symptoms in the part of the world's population living on grains, such as rice as the major or almost only staple food. In south-east Asia alone, it is estimated that 5 million children develop the eye disease xerophthalmia every year, of which 0.25 million eventually go blind (Sommer, 1988; Grant, 1991). Furthermore, although vitamin A deficiency is not a proximal determinant of death, it is correlated with an increased susceptibility to potential fatal afflictions such as diarrhea, respiratory diseases and childhood diseases, such as measles (Grant, 1991). According to statistics compiled by UNICEF, improved provitamin nutrition could prevent 1-2 million deaths annually among children aged 1-4 years, and an additional 0.25-0.5 million deaths during later childhood (Humphrey et al., 1992). For these reasons it is very desirable to raise the total carotenoid levels in staple foods. Moreover, carotenoids are known to assist in the prevention of several sorts of cancer and the role of lutein and zeaxanthin in the retina preventing macular degeneration is established (see e.g. Brown et al., 1998; Schalch, 1992). There is also a need to provide increased β carotene in so called “orphan crops” such as sorghum, cassaya, millet, sweet potato and cowpea, which are relied upon heavily in Africa. In terms of tonnage, sorghum is Africa's second most important cereal.

The continent produces about 20 million tons of sorghum per annum, about one-third of the world crop. However, these figures do not do justice to the importance of sorghum in Africa. It is the only viable food grain for many of the world's most food insecure people, and what's more sorghum is uniquely adapted to Africa's climate, being both drought resistant and able to withstand periods of water-logging.

Furthermore, carotenoids have a wide range of applications as colorants in human food and animal feed as well as in pharmaceuticals. In addition there is increasing interest in carotenoids as nutriceutical compounds in “functional food”. This is because some carotenoids, e.g. β-carotene, exhibit provitamin-A character in mammals.

Many attempts have been made over the years to alter or enhance carotenoid biosynthetic pathways in various plant tissues such as vegetative tissues or seeds, or in bacteria. (See, for example, WO 96/13149, WO 98/06862, WO 98/24300, WO 96/28014, and U.S. Pat. No. 5,618,988). Recently applications aiming at de novo carotenoid biosynthesis in plant material essentially carotenoid-free, such as rice endosperm have resulted in rice with increased β carotene levels, referred to as golden rice (Ye et al., Science 287:303-5, 2000; Paine J et. al., Nature Biotechnology (2005) 4:482-487; Beyer P et al., The Journal of Nutrition 132: 506S-510S, 2002; U.S. Pat. No. 7,838,749). However, it has been reported that the β carotene in golden rice has a relatively short half-life in grain stored at ambient temperatures. Similarly, overexpressing enzymes involved in carotenoid biosynthesis in sorghum have resulted in increased β carotene levels but a half-life of only 4 weeks at ambient temperature. More recently applications aiming at altering carotenoid biosynthesis in oil-rich seeds have resulted in increased β carotene levels (WO2000/53768; WO2004/085656).

It is apparent that there are still needed methods for further increasing β carotene accumulation levels by expressing other enzymes in carotenoid biosynthesis pathway necessary to produce carotenes and xanthophylls of interest in other crops, such as sorghum and means to increase β carotene half-life, bioaccessibility and bioavailability in grain, particularly sorghum.

### SUMMARY OF THE INVENTION

The present disclosure provides means and methods of transforming plant cells, seeds, tissues or whole plants in order to yield transformants capable of expressing enzymes of the vitamin E biosynthesis pathway (FIG. 1) to increase the half-life of carotenoids, particularly β carotene, and to increase the bioaccessibility and bioavailability of carotenoids, particularly β carotene in plant parts, particularly grain. The present disclosure also provides means and methods of transforming plant cells, seeds, tissues or whole plants in order to yield transformants capable of expressing all enzymes of the methylerythritol phosphate (MEP) biosynthesis pathway (FIG. 2) that are involved in the biosynthesis of the isopentyl pyrophosphate (IPP) and dimethylallyl diphosphate (DMAPP) intermediates used by geranylgeranyl pyrophosphate synthase to form geranylgeranyl pyrophosphate (GGPP). The present disclosure also provides means and methods of transforming plant cells, seeds, tissues or whole plants in order to yield transformants capable of expressing all enzymes of the carotenoid biosynthesis pathway (FIG. 3) that are essential for the targeted host plant to accumulate carotenes and/or xanthophylls of interest. The present disclosure also provides DNA molecules designed to be suitable for carrying out the method of the disclosure, and plasmids or vector systems comprising said molecules. Furthermore, the present disclosure provides transgenic plant cells, seeds, tissues and whole plants that display an improved nutritional quality and contain such DNA molecules and/or that have been generated by use of the methods of the present disclosure.

The present disclosure also provides both the de novo introduction and expression of vitamin E biosynthesis and the modification of pre-existing vitamin E biosynthesis in order to up- or down-regulate accumulation of certain intermediates of vitamin E biosynthesis products of interest. The present disclosure also provides both the de novo introduction and expression of carotenoid biosynthesis and/or methylerythritol phosphate (MEP) biosynthesis, which is particularly important with regard to plant material that is known to be essentially carotenoid-free, such as the seeds of many cereals, and the modification of pre-existing carotenoid biosynthesis and/or methylerythritol phosphate (MEP) biosynthesis in order to up-or down-regulate accumulation of certain intermediates of carotenoid biosynthesis products of interest.

The following embodiments are encompassed by the present disclosure.

1. A recombinant DNA molecule, comprising
a first exogenous expression cassette capable of directing production in a plant cell of at least one enzyme in the carotenoid synthesis pathway; and
a second exogenous expression cassette capable of directing production in a plant cell of at least one enzyme in the tocochromanol synthesis pathway.
   2. The recombinant DNA molecule according to embodiment 1, wherein the enzyme in the tocochromanol synthesis pathway is a homogentisate geranylgeranyl transferase (HGGT).
   3. The recombinant DNA molecule according to embodiment 2, wherein the homogentisate geranylgeranyl transferase is derived from Hordeum vulgare, Zea mays, Glycine max or Arabidopsis thaliana.
   4. The recombinant DNA molecule according to embodiment 3, wherein the homogentisate geranylgeranyl transferase is derived from Hordeum vulgare.
   5. The recombinant DNA molecule according to any one of embodiments 1, 2, 3 or 4, wherein the plant cell has increased tocopherol/tocotrienol levels compared to a plant cell not having the second exogenous expression cassette.
   6. The recombinant DNA molecule according to any one of embodiments 1, 2, 3, 4 or 5, further comprising an exogenous expression cassette capable of directing production in the cell of at least one enzyme in the methylerythritol phosphate biosynthesis pathway.
   7. The recombinant DNA molecule according to embodiment 6, wherein the at least one enzyme in the methylerythritol phosphate biosynthesis pathway is D-1-deoxy-xylulose 5-phosphate synthase (DXS).
   8. The recombinant DNA molecule according to embodiment 7, wherein the D-1-deoxy-xylulose 5-phosphate synthase is derived from Arabidopsis thaliana.
   9. The recombinant DNA molecule according to any one of embodiments 1, 2, 3, 4, 5, 6, 7 or 8, wherein the at least one enzyme in the carotenoid synthesis pathway is a phytoene synthase (PSY).
   10. The recombinant DNA molecule according to embodiment 9, wherein the phytoene synthase is derived from Zea mays.
   11. The recombinant DNA molecule according to embodiment 10, wherein the phytoene synthase is derived from Zea mays PSY1 or PSY3.
   12. The recombinant DNA molecule according to any one of embodiments 1, 2, 3, 4, 5, 6, 7, 8, 9, 10 or 11, wherein the at least one enzyme in the carotenoid synthesis pathway is a phytoene desaturase (carotenoid reductase (CRT).
   13. The recombinant DNA molecule according to embodiment 12, wherein the carotenoid reductase (CRT) is derived from Erwinia uredovora.
   14. The recombinant DNA molecule according to any one of embodiments 12 or 13 wherein the carotenoid reductase (CRT) is operably linked with a suitable plastid transit peptide.
   15. The recombinant DNA molecule according to embodiment 14, wherein the transit peptide is derived from Pisum sativum ribulose-1,5-bisphosphate carboxylase.
   16. The recombinant DNA molecule according to any one of embodiments 1, 2, 3, 4, 5, 6, 7, 8, 9, 10, 11, 12, 13, 14 or 15, wherein the homogentisate geranylgeranyl transferase (HGGT) operably linked to a tissue specific or constitutive promoter.
   17. The recombinant DNA molecule according to embodiment 16 wherein the homogentisate geranylgeranyl transferase (HGGT) is operably linked to a tissue specific promoter.
   18. The recombinant DNA molecule according to embodiment 17 wherein the tissue specific promoter is an endosperm preferential promoter.
   19. The recombinant DNA molecule according to embodiment 18 wherein the endosperm preferential promoter is derived from Sorghum bicolor alpha kafirin A1 gene.
   20. The recombinant DNA molecule according to any one of embodiments 7 or 8, wherein the D-1-deoxy-xylulose 5-phosphate synthase is operably linked to a tissue specific or constitutive promoter.
   21. The recombinant DNA molecule according to embodiment 20, wherein the D-1-deoxy-xylulose 5-phosphate synthase is operably linked to a tissue specific promoter.
   22. The recombinant DNA molecule according to embodiment 21, wherein the tissue specific promoter is an endosperm preferential promoter.
   23. The recombinant DNA molecule according to embodiment 22, wherein the endosperm preferential promoter is derived from Zea mays 27 kD gamma zein gene.
   24. The recombinant DNA molecule according to any one of embodiments 9, 10 or 11, wherein the phytoene synthase is operably linked to a tissue specific or constitutive promoter.
   25. The recombinant DNA molecule according to embodiment 24, wherein the phytoene synthase is operably linked to a tissue specific promoter.
   26. The recombinant DNA molecule according to embodiment 25, wherein the tissue specific promoter is an endosperm preferential promoter.
   27. The recombinant DNA molecule according to embodiment 26, wherein the endosperm preferential promoter is derived from Sorghum bicolor alpha kafirin B1 gene.
   28. The recombinant DNA molecule according to any one of embodiments 12, 13, 14, or 15, wherein the phytoene desaturase is operably linked to a tissue specific or constitutive promoter.
   29. The recombinant DNA molecule according to embodiment 28, wherein the phytoene desaturase is operably linked to a tissue specific promoter.
   30. The recombinant DNA molecule according to embodiment 29, wherein the tissue specific promoter is an endosperm preferential promoter.
   31. The recombinant DNA molecule according to embodiment 30, wherein the endosperm preferential promoter is derived from Sorghum bicolor beta kafirin gene.
   32. The recombinant DNA molecule of any one according to embodiments 1-31, wherein the at least one recombinant DNA further comprises a polynucleotide encoding a selectable marker.
   33. The recombinant DNA molecule according to any one of embodiments 1-32, further comprising an exogenous expression cassette capable of directing production in the cell of at least one carotenoid-associated protein.
   34. The recombinant DNA molecule according to any one of embodiments 1-32, further comprising an exogenous expression cassette capable of directing production in the cell of at least one Orange (Or) mutant gene.
   35. An expression vector, comprising
a first recombinant polynucleotide encoding at least one enzyme in the carotenoid synthesis pathway operably linked to at least one regulatory element;
a second recombinant polynucleotide encoding at least one enzyme in the tocochromanol synthesis pathway operably linked to at least one regulatory element.
   36. The expression vector according to embodiment 35, wherein the enzyme in the tocochromanol synthesis pathway is a homogentisate geranylgeranyl transferase (HGGT).
   37. The expression vector according to embodiment 36, wherein the homogentisate geranylgeranyl transferase is derived from Hordeum vulgare, Zea mays, Glycine max or Arabidopsis thaliana.
   38. The expression vector according to embodiment 37, wherein the homogentisate geranylgeranyl transferase is derived from Hordeum vulgare.
   39. The expression vector according to any one of embodiments 35, 36, 37 or 38 wherein the expression vector further comprises recombinant polynucleotide encoding least one enzyme in the methylerythritol phosphate biosynthesis pathway operably linked to at least one regulatory element.
   40. The expression vector according to embodiment 39, wherein the at least one enzyme in the methylerythritol phosphate biosynthesis pathway is D-1-deoxy-xylulose 5-phosphate synthase (DXS).
   41. The expression vector according to embodiment 40, wherein the D-1-deoxy-xylulose 5-phosphate synthase is derived from Arabidopsis thaliana.
   42. The expression vector according to any one of embodiments 35, 36, 37, 38, 39, 40 or 41 wherein the at least one enzyme in the carotenoid synthesis pathway is a phytoene synthase (PSY).
   43. The expression vector according to embodiment 42, wherein the phytoene synthase is derived from Zea mays.
   44. The expression vector according to embodiment 43, wherein the phytoene synthase is derived from Zea mays PSY1 or PSY3.
   45. The expression vector according to any one of embodiments 35, 36, 37, 38, 39, 40, 41, 42, 43 or 44 wherein the at least one enzyme in the carotenoid synthesis pathway is a phytoene desaturase (carotenoid reductase (CRT).
   46. The expression vector according to embodiment 45, wherein the carotenoid reductase (CRT) is derived from Erwinia uredovora.
   47. The expression vector according to any one of embodiments 45 or 46, wherein the carotenoid reductase (CRT) is operably linked with a suitable plastid transit peptide.
   48. The expression vector according to embodiment 47, wherein the transit peptide is derived from Pisum sativum ribulose-1,5-bisphosphate carboxylase.
   49. The expression vector according to any one of embodiments 35, 36, 37, 38, 39, 40, 41, 42, 43, 44, 45, 46, 47 or 48 wherein the at least one regulatory element operably linked to the homogentisate geranylgeranyl transferase (HGGT) is a tissue specific or constitutive promoter.
   50. The expression vector according to embodiment 49, wherein the at least one regulatory element a tissue specific promoter.
   51. The expression vector according to embodiment 50, wherein the tissue specific promoter is an endosperm preferential promoter.
   52. The expression vector according to embodiment 51, wherein the endosperm preferential promoter is derived from Sorghum bicolor alpha kafirin A1 gene.
   53. The expression vector according to any one of embodiments 40 or 41, wherein the at least one regulatory element operably linked to the D-1-deoxy-xylulose 5-phosphate synthase is a tissue specific or constitutive promoter.
   54. The expression vector according to embodiment 53, wherein the at least one regulatory element is a tissue specific promoter.
   55. The expression vector according to embodiment 54, wherein the tissue specific promoter is an endosperm preferential promoter.
   56. The expression vector according to embodiment 55, wherein the endosperm preferential promoter is derived from Zea mays 27 kD gamma zein gene.
   57. The expression vector according to any one of embodiments 42, 43 or 44, wherein the at least one regulatory element operably linked to the phytoene synthase is a tissue specific or constitutive promoter.
   58. The expression vector according to embodiment 57, wherein the at least one regulatory element is a tissue specific promoter.
   59. The expression vector according to embodiment 58, wherein the tissue specific promoter is an endosperm preferential promoter.
   60. The expression vector according to embodiment 59, wherein the endosperm preferential promoter is derived from Sorghum bicolor alpha kafirin B1 gene.
   61. The expression vector according to any one of embodiments 45, 46, 47 or 48 wherein the at least one regulatory element operably linked to the phytoene desaturase is a tissue specific or constitutive promoter.
   62. The expression vector according to embodiment 61, wherein the at least one regulatory element is a tissue specific promoter.
   63. The expression vector according to embodiment 62, wherein the tissue specific promoter is an endosperm preferential promoter.
   64. The expression vector according to embodiment 63, wherein the endosperm preferential promoter is derived from Sorghum bicolor beta kafirin gene.
   65. The expression vector according to any one of embodiments 35-64, further comprising a polynucleotide encoding a selectable marker.
   66. The expression vector according to any one of embodiments 35-64, further comprising a recombinant polynucleotide encoding at least one carotenoid-associated protein operably linked to at least one regulatory element.
   67. The expression vector according to any one of embodiments 35-64, further comprising a recombinant polynucleotide encoding at least one Orange (Or) mutant gene operably linked to at least one regulatory element.
   68. A method of increasing total carotenoid levels and/or increasing carotenoid half-life in a plant, comprising
transforming a plant cell with the recombinant DNA molecule of any one according to embodiments 1-34; and
selecting a transformed plant that comprises the cells having increased total carotenoid accumulation and/or increased carotenoid stability compared to a plant cell not having the second exogenous expression cassette.
   69. The method according to embodiment 68, wherein the carotenoid is β-carotene.
   70. The method according to claim 68 or 69, wherein the transformed plant has increased tocopherol/tocotrienols levels compared to a plant cell not having the second exogenous expression cassette.
   71. A method of increasing total carotenoid levels and/or increasing carotenoid half-life in a plant, comprising
transforming a plant cell with the expression vector of any one according to embodiments 35-67; and
selecting a transformed plant that comprises the cells having increased carotenoid accumulation and/or increased beta carotene stability compared to a plant cell not having the expression vector.
   72. The method according to embodiment 71, wherein the carotenoid is β-carotene.
   73. The method according to claim 71 or 72, wherein the transformed plant has increased tocopherol/tocotrienols levels compared to a plant cell not having the expression vector.
   74. The method according to any one of embodiments 64, 65, 66, 67, 68 or 69, wherein the plant is sorghum.
   75. A transgenic plant or progeny thereof, comprising the recombinant polynucleotide molecule of any one according to embodiments 1-32.
   76. A transgenic plant or progeny thereof, comprising the expression vector of any one according to embodiments 33-63.
   77. The transgenic plant or progeny thereof of embodiment 75 or 76, wherein the plant is sorghum.
   78. Seed, grain or processed product thereof of the transgenic plant according to any one of embodiments 70 or 71, wherein the seed, grain or processed product thereof has increased carotenoid levels and/or carotenoid stability.
   79. A method of increasing carotenoid bioavailability in grain, comprising
expressing in a transgenic plant at least one exogenous enzyme in the carotenoid synthesis pathway in a seed specific manner; and
expressing in a transgenic plant at least one exogenous enzyme in the tocochromanol synthesis pathway in a seed specific manner,
wherein the grain has increased carotenoid bioavailability compared to grain not expressing the enzyme in the tocochromanol synthesis pathway in a seed specific manner.
   80. The method according to embodiment 79, wherein the enzyme in the tocochromanol synthesis pathway is a homogentisate geranylgeranyl transferase (HGGT).
   81. The method according to embodiment 80, wherein the homogentisate geranylgeranyl transferase is derived from Hordeum vulgare, Zea mays, Glycine max or Arabidopsis thaliana.
   82. The method according to embodiment 81, wherein the homogentisate geranylgeranyl transferase is derived from Hordeum vulgare.
   83. The method according to any one of embodiments 79, 80, 81 or 82, wherein the grain has increased tocopherol/tocotrienol levels compared to grain from a plant not expressing the exogenous enzyme in the tocochromanol synthesis pathway in a seed specific manner.
   84. The method according to any one of embodiments 80, 81, 82 or 83, wherein the method further comprises expressing at least one enzyme in the methylerythritol phosphate biosynthesis pathway in a seed specific manner.
   85. The method according to embodiment 84, wherein the at least one enzyme in the methylerythritol phosphate biosynthesis pathway is D-1-deoxy-xylulose 5-phosphate synthase (DXS).
   86. The method according to embodiment 85, wherein the D-1-deoxy-xylulose 5-phosphate synthase is derived from Arabidopsis thaliana.
   87. The method according to any one of embodiments 79, 80, 81, 82, 83, 84, 85 or 86 wherein the at least one enzyme in the carotenoid synthesis pathway is a phytoene synthase (PSY).
   88. The method according to embodiment 87, wherein the phytoene synthase is derived from Zea mays.
   89. The method according to embodiment 88, wherein the phytoene synthase is derived from Zea mays PSY1 or PSY3.
   90. The method according to any one of embodiments 79, 80, 81, 82, 83, 84, 85, 86, 87, 88 or 89 wherein the at least one enzyme in the carotenoid synthesis pathway is a phytoene desaturase (carotenoid reductase (CRT).
   91. The method according to embodiment 90, wherein the carotenoid reductase (CRT) is derived from Erwinia uredovora.
   92. The method according to any one of embodiments 90 or 9 wherein the carotenoid reductase (CRT) is operably linked with a suitable plastid transit peptide.
   93. The method according to embodiment 92, wherein the transit peptide is derived from Pisum sativum ribulose-1,5-bisphosphate carboxylase.
   94. The method according to any one of embodiments 79, 80, 81, 82, 83, 84, 85, 86, 87, 88, 89, 90, 91, 92 or 93 wherein the homogentisate geranylgeranyl transferase (HGGT) expressed in an endosperm specific manner.
   95. The method according to any one of embodiments 85 or 86, wherein the D-1-deoxy-xylulose 5-phosphate synthase is expressed in an endosperm specific manner.
   96. The method according to any one of embodiments 87, 88 or 89, wherein the phytoene synthase is expressed in an endosperm specific manner.
   97. The method according to any one of embodiments 90, 91, 92 or 93, wherein the phytoene desaturase is expressed in an endosperm specific manner.
   98. The method according to any one of embodiments 79-97, wherein the method further comprises expressing at least one carotenoid-associated protein.
   99. The method according to any one of embodiments 79-98, wherein the method further comprises expressing at least one Orange (Or) mutant gene.

### ABBREVIATIONS USED THROUGHOUT THE SPECIFICATION

The systematic names of relevant carotenoids mentioned herein are:
Phytoene: 7,8,11,12,7′,8′,11′,12′-octahydro-φ,φ-carotene
Phytofluene: 7,8,11,12,7′,8′,-hexahydro-φ,φ-carotene
ζ-carotene: 7,8,7′,8′-tetrahydro-φ,φ-carotene
Neurosporene: 7,8,-dihydro-φ,φ-carotene
Lycopene: φ,φ-carotene
β-carotene: β,β-carotene
α-carotene: β,ε-carotene
Zeaxanthin: β,β,carotene-3,3′-diol
Lutein: β,ε-carotene-3,3′-diol
Antheraxanthin: 5,6-epoxy-5,6-dihydro-β,β,carotene-3,3′-diol
Violaxanthin: 5,6,5′,6′-diepoxy-5,6,5′,6′,tetrahydro-β,β,carotene-3,3′-diol
Neoxanthin:5′,6′-epoxy-6,7-didehdro-5,6,5′,6′-tetrahydro-β,β,carotene-3,5,3′-triol

Enzymes:
PSY: phytoene synthase
PDS: phytoene desaturase
Crt-I: bacterial carotene desaturase
ZDS: ζ (zeta)-carotene desaturase
DXS: deoxyxylulose phosphate synthase
HGGT: homogentisate geranylgeranyl transferase
CYC:lycopene βcyclase

Non-Carotene Intermediates:
IPP: isopentenyl diphosphate
DMAPP: dimethylallyl-diphosphate
GGPP: geranylgeranyl diphosphate

As used herein, the term “plant” generally includes eukaryotic alga, embryophytes including Bryophyte, Pteridoplyta and Spermatophyta such as Gymnospermae and Angiospermae, the latter including Magnoliopsida, Rosopsida (eu-“dicots”), Liliopsida (“monocots”). Representative examples include grain seeds, e.g. rice, wheat, barley, oats, amaranth, flax, triticale, rye, corn, sorghum, and other grasses; oil seeds, such as oilseed Brassica seeds, cotton seeds, soybean, safflower, sunflower, coconut, palm, and the like; other edible seeds or seeds with edible parts including pumpkin, squash, sesame, poppy, grape, mung beans, peanut, peas, beans, radish, alfalfa, cocoa, coffee, hemp, tree nuts such as walnuts, almonds, pecans, chick-peas etc. Furthermore, potatoes carrots, sweet potatoes, tomato, pepper, cassaya, willows, oaks, elm, maples, apples, bananas; ornamental flowers such as lilies, orchids, sedges, roses, buttercups, petunias, phlox, violets, sunflowers, and the like. Generally, the present disclosure is applicable in ornamental species as well as species cultivated for food, fiber, wood products, tanning materials, dyes, pigments, gums, resins, latex products, fats, oils, drugs, beverages, and the like. In some embodiments the target plant selected for transformation is cultivated for food, including but not limited to, grains, roots, legumes, nuts, vegetables, tubers, fruits, spices and the like.

### BRIEF DESCRIPTION OF THE DRAWINGS

FIG. 1 shows the tocochromanols biosynthesis pathway in plants.

FIG. 2 shows the methylerythritol phosphate (MEP) biosynthesis pathway in plants.

FIG. 3 shows the carotenoid biosynthesis pathway in plants.

FIG. 4 shows the branching of serving several different biosynthetic pathways from geranylgeranyl diphosphate (GGPP).

FIG. 5 shows the percent β carotene versus control (no treatment) of air and oxygen induced degradation of β carotene after four weeks at room temperature in ABS168 seeds from T2 sorghum plants.

FIG. 6 shows the β carotene levels (μg/g) of seeds from T2 sorghum plants from 13 ABS203 events.

FIG. 7 shows the relationships between β-carotene (μg/g) and γ-tocopherol levels (μg/g) according to the correlation coefficient for T1 plants from the 13 ABS203 events.

FIG. 8 illustrates the degradation of β-carotene (log(% β-carotene relative to control) in ABS198 and ABS203 seeds at 9% O₂ under vacuum, 20% O₂ and 100% O₂ for four weeks at room temperature.

FIG. 9 panel (a) illustrates the t₁₁₂ (weeks) of β carotene of ABS198 and ABS203 sorghum T1 mixed homozygous, hemizygous, and null seeds and panel (b) illustrates the t_{1/2} (weeks) of β carotene of ABS198 and ABS203 T1 seeds.

FIG. 10 shows the relative phytoene synthase (PSY1) level in ABS203 T3 seeds (solid line) and the β-carotene levels (μg/g) in T2 ABS198 (♦) and T3 ABS203 (▪) seeds at 10 to 40 days after pollination and at maturity (50 days).

FIG. 11 shows the 100 seed weights and the β carotene levels (μg/g) of the 13 ABS203 events.

FIG. 12 shows the total β carotene bioavailability by Caco-2 cell analysis of ABS188 events.

FIG. 13 shows the correlation between Yield (g/3 ft. of row) and β-carotene (ug/g) for thirteen ABS203 homozygous sorghum plants.

FIG. 14 shows the correlation between the percentage of seeds germination and β-carotene (ug/g) for thirteen ABS203 homozygous sorghum plants.

FIG. 15 shows the Yield (g/3 ft. of row) and β-carotene (ug/g) for five ABS203 homozygous sorghum plants compared to null sorghum plants.

### DETAILED DESCRIPTION OF THE INVENTION

Carotenoid Biosynthesis

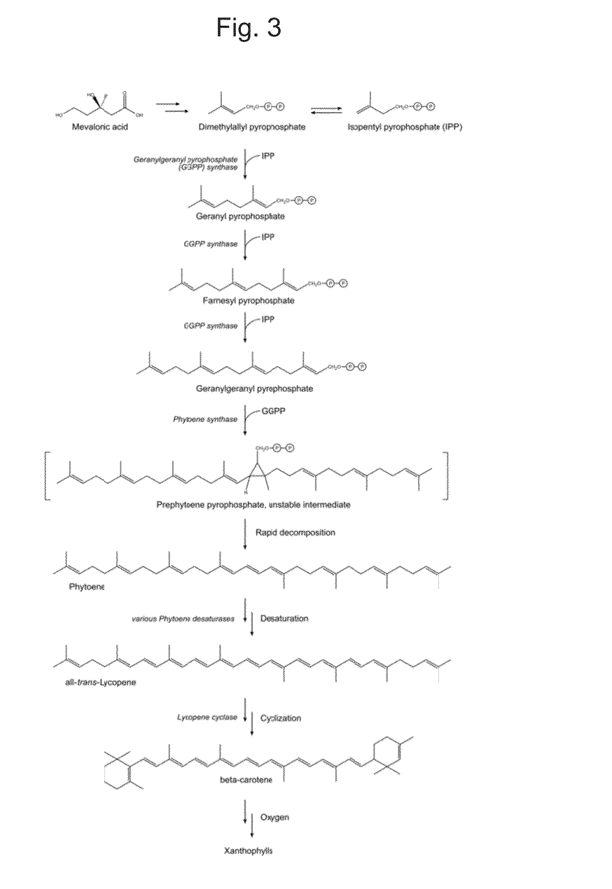

Carotenoids are 40-carbon (O₄₀) isoprenoids formed by condensation of eight isoprene units derived from the biosynthetic precursor isopentenyl diphosphate (IPP) (see FIG. 3). By nomenclature, carotenoids fall into two classes, namely carotenes, comprising hydrocarbons whereas oxygenated derivatives are referred to as xanthophylls. Their essential function in plants is to protect against photo-oxidative damage in the photosynthetic apparatus of plastids. In addition they participate in light harvesting during photosynthesis and represent integral components of photosynthetic reaction centers. Carotenoids are the direct precursors of the phytohormone abscisic acid.

Carotenoid biosynthesis as schematically depicted in FIG. 3 has been investigated and the pathway has been elucidated in bacteria, fungi and plants (see for example, Britton, 1988). In plants, carotenoids are formed in plastids. The early intermediate of the carotenoid biosynthetic pathway is geranylgeranyl diphosphate (GGPP); formed by the enzyme geranylgeranyl diphosphate synthase from isopentenyl diphosphate (IPP) and dimethylallyl diphosphate (DMAPP) (see FIG. 3). Phytoene synthase (PSY) catalyzes the first committed step in carotenogenesis by condensation of two molecules of geranylgeranyl pyrophosphate (GGPP) to form phytoene (for review, see Matthews and Wurtzel, 2007). This enzymatic step has been found to be rate limiting in several different plant species, tissues and developmental states. The expression levels of PSY appear to be closely correlated with the level of carotenoids (Giuliano, Bartley & Scolnik 1993). The subsequent enzymatic step, also representing the first carotenoid-specific reaction, is catalyzed by the enzyme. The reaction comprises a two-step reaction resulting in a head-to head condensation of two molecules of GGPP to form the first, yet uncolored carotene product, phytoene (Dogbo et al., 1988, Chamovitz et al., 1991; Linden et al., 1991; Pecker et al., 1992). Phytoene synthase occurs in two forms soluble/inactive and membrane-bound/active and it requires vicinal hydroxy functions for activity as present in the surface of plastid galactolipid-containing membranes (Schledz et al., 1996).

While the formation of phytoene is similar in bacteria and plants, the metabolization of phytoene differs pronouncedly. In plants, two gene products operate in a sequential manner to generate the colored carotene lycopene (Beyer et al., 1989). They are represented by the enzymes phytoene desaturase (PDS, see e.g. Hugueney et al., 1992) and ζ-carotene desaturase (ZDS, see e.g. Albrecht et al., 1996). Each introduces two double bonds yielding ζ-carotene via phytofluene and lycopene via neurosporene, respectively. PDS is believed to be mechanistically linked to a membrane-bound redox chain (Nievelstein et al., 1995) employing plastoquinone (Mayer et al., 1990; Schulz et al., 1993; Norris et al., 1995), while ZDS acts mechanistically in a different way (Albrecht et al., 1996). In plants, the entire pathway seems to involve cis-configured intermediates (Bartley et al., 1999). In contrast, in many bacteria, such as in the genus Erwinia, the entire desaturation sequence forming all four double bonds is achieved by a single gene product (Crt I), converting phytoene to lycopene directly (see e.g. Miawa et al., 1990; Armstrong et al., 1990, Hundle et al., 1994). Erwinia uredovora phytoene desaturase (Crt I) converts phytoene to lycopene, a step that requires three plant enzymes, phytoene desaturase (PDS), ζ-carotene desaturase (ZDS), and carotene cis-transisomerase (CRTISO) to work sequentially. This type of bacterial desaturase is known not to be susceptible to certain bleaching herbicides which efficiently inhibit plant-type phytoene desaturase.

In plants, two gene products catalyze the cyclization of lycopene, namely α (ε)- and β-lycopene cyclases, forming α(ε)- and β-ionone end-groups, respectively (see e.g. Cunningham et al., 1993; Scolnik and Bartley, 1995, Cunningham et al., 1996). In plants, normally β-carotene carrying two β-ionone end-groups and α-carotene, carrying one α(ε) and one β-ionone end-group are formed.

The formation of the plant xanthophylls is mediated first by two gene products, α- and β-hydroxylases (Masamoto et al., 1998) acting in the position C3 and C3′ of the carotene backbone of α- and β-carotene, respectively. The resulting xanthophylls are named lutein and zeaxanthin.

Further oxygenation reactions are catalyzed by zeaxanthin epoxydase catalyzing the introduction of epoxy-functions in position C5,C6 and C5′,C6′ of the zeaxanthin backbone (Marin et al., 1996). This leads to the formation of antheraxanthin and violaxanthin. The reaction is made reversible by the action of a different gene product, violaxanthin de-epoxydase (Bugos and Yamamoto, 1996). Neoxanthin synthase leading to the formation of neoxanthin has been identified from tomato (Bouvier F et. al. Eur J Biochem (2000) 267(21):6346-52); and potato (Al-Bablil S. et. al., FEBS Letters (2000) 485:168-172; Arabidopsis thaliana (Ferro, M. Molecular & Cellular Proteomics (2003) 2:325-345).

Genes and cDNAs coding for carotenoid biosynthesis genes have been cloned from a variety of organisms, ranging from bacteria to plants. Bacterial and cyanobacterial genes include Erwinia herbicola (Application WO 91/13078, Armstrong et al., 1990), Erwinia uredovora (Misawa et al., 1990), Erwinia uredovora (now Pantoea anantis; CRT B-GenBank accession: D90087.2) R. capsulatus (Armstrong et al., 1989), Thermus thermophilus (Hoshino et al., 1993), the cyanobacterium Synechococcus sp. (GenBank accession number X63873), Flavobacterium sp. strain R1534 (Pasamontes et al., 1997), and Panteoa agglomeras (U.S. Pat. No. 6,929,928). Genes and cDNAs coding for enzymes in the carotenoid biosynthetic pathway in higher plants have been cloned from various sources, including Arabidopsis thaliana, Sinalpis alba, Capsicuin annuum, Naricisstis pseudonarcissus, Lycopersicon esculentum, etc., as can be deduced from the public databases. IPP isomerase has been isolated from: R. Capsulatus (Hahn et al. (1996) J. Bacteriol. 178:619-624 and the references cited therein), GenBank Accession Nos. U48963 and X82627, Clarkia xantiana GenBank Accession No. U48962, Arabidopsis thaliana GenBank Accession No. U48961, Schizosaccharmoyces pombe GenBank Accession No. U21154, human GenBank Accession No. X17025, Kluyveromyces lactis GenBank Accession No. X14230. Geranylgeranyl pyrophosphate synthase has been isolated from: E. Uredovora Misawa et al. (1990) J. Bacteriol. 172:6704-6712 and Application WO 91/13078; and from plant sources, including white lupin (Aitken et al. (1995) Plant Phys. 108:837-838), bell pepper (Badillo et al. (1995) Plant Mol. Biol. 27:425-428) and Arabidopsis (Scolnik and Bartely (1994) Plant Physiol 104:1469-1470; Zhu et al. (1997) Plant Cell Physiol. 38:357-361). Phytoene synthase has been isolated from: a number of sources including E. Uredovora, Rhodobacter capsulatus, and plants Misawa et al. (1990) J. Bacteriol. 172:6704-6712, GenBank Accession No. D90087, Application WO 91/13078, Armstrong et al. (1989) Mol. Gen. Genet. 216:254-268, Armstrong, G. A. “Genetic Analysis and regulation of carotenoid biosynthesis. In R. C. Blankenship, M. T. Madigan, and C. E. Bauer (ed.), Anoxygenic photosynthetic bacteria; advances in photosynthesis. Kluwer Academic Publishers, Dordrecht, The Netherlands, Armstrong et al. (1990) Proc. Natl. Acad Sci USA 87:9975-9979, Armstrong et al. (1993) Methods Enzymol. 214:297-311, Bartley and Scolnik (1993) J. Biol. Chem. 268:27518-27521, Bartley et al. (1992) J. Biol. Chem. 267:5036-5039, Bramley et al. (1992) Plant J. 2:291-343, Ray et al. (1992) Plant Mol. Biol. 19:401-404, Ray et al. (1987) Nucleic Acids Res. 15:10587, Romer et al. (1994) Biochem. Biophys. Res. Commun. 196:1414-1421, Karvouni et al. (1995) Plant Molecular Biology 27:1153-1162, GenBank Accession Nos. U32636, Z37543, L37405, X95596, D58420, U32636, Z37543, X78814, X82458, S71770, L27652, L23424, X68017, L25812, M87280, M38424, X69172, X63873, and X60441, Armstrong, G. A. (1994) J. Bacteriol. 176:4795-4802 and the references cited therein. Phytoene desaturase has been isolated from: bacterial sources including E. uredovora Misawa et al. (1990) J. Bacteriol. 172:6704-6712, and Application WO 91/13078 (GenBank Accession Nos. L37405, X95596, D58420, X82458, S71770, and M87280); and from plant sources, including maize (Li et al. (1996) Plant Mol. Biol. 30:269-279), tomato (Pecker et al. (1992) Proc. Nat. Acad. Sci. 89:4962-4966 and Aracri et al. (1994) Plant Physiol. 106:789), and Capisum annuum (bell peppers) (Hugueney et al. (1992) J. Biochem. 209: 399-407), GenBank Accession Nos. U37285, X59948, X78271, and X68058).

A number of other carotenoid biosynthesis enzymes have also been isolated including but not limited to: β-carotene hydroxylase or crtZ (Hundle et al. (1993) FEBS Lett. 315:329-334, GenBank Accession No. M87280; U.S. Pat. No. 2,008,0276331) for the production of zeaxanthin; genes encoding keto-introducing enzymes, such as crtW (Misawa et al. (1995) J. Bacteriol. 177:6575-6584, WO 95/18220, WO 96/06172) or β-C-4-oxygenzse (crtO; Harker and Hirschberg (1997) FEBS Lett. 404:129-134) for the production of canthaxanthin; crtZ and crtW or crtO for the production of astaxanthin; ε-cyclase and ε-hydroxylase for the production of lutein; ε-hydroxylase and crtZ for the production of lutein and zeaxanthin; antisense lycopene ε-cyclase (GenBank Accession No. U50738) for increased production of β-carotene; antisense lycopene ε-cyclase and lycopene ε-cyclase (Hugueney et al. (1995) Plant J. 8:417-424, Cunningham F X Jr (1996) Plant Cell 8:1613-1626, Scolnik and Bartley (1995) Plant Physiol. 108:1343, GenBank Accession Nos. X86452, L40176, X81787, U50739 and X74599) for the production of lycopene; and antisense plant phytoene desaturase for the production of phytoene. In this manner, the pathway can be modified for the high production of any particular carotenoid compound of interest. Such compounds include but are not limited to α-cryptoxanthin, β-cryptoxanthin, ζ-carotene, phytofluene, neurosporane, and the like. Using the methods of the invention, any compound of interest in the carotenoid pathway can be produced at high levels in a seed.

The pathway can also be manipulated to decrease levels of a particular carotenoid by transformation of antisense DNA sequences which prevent the conversion of the precursor compound into the particular carotenoid being regulated. See, generally, Misawa et al. (1990) J. of Bacteriology 172:6704-6712, E.P. 0393690 B1, U.S. Pat. No. 5,429,939, Bartley et al. (1992) J. Biol. Chem. 267:5036-5039, Bird et al. (1991) Biotechnology 9:635-639, and U.S. Pat. No. 5,304,478, which disclosures are herein incorporated by reference.

The expression of phytoene synthase from tomato can affect carotenoid levels in fruit (Bird et al., 1991; Brarley et al., 1992; Fray and Grier-son, 1993). Over-expression of ZM-PSY1 in maize increases the level of this biosynthetic protein resulting in elevated production of Vitamin A. (Naqvi S., et al. PNAS 12:7762-7767 2009). It has also been reported that constitutive expression of a phytoene synthase in transformed tomato plants results in dwarfism, due to redirecting the metabolite GGPP from the gibberellin biosynthetic pathway (Fray et al., 1995). No such problems were noted upon constitutively expressing phytoene synthase from Narcissus pseudonarcissus in rice endosperm (Burkhardt et al., 1997). Erwinia uredovora Crt I, as a bacterial desaturase, is known to function in plants and to confer bleaching-herbicide resistance (Misawa et al., 1993).

In accordance with the subject disclosure, means and methods of transforming plant cells, seeds, tissues or whole plants are provided to produce transformants capable of expressing all enzymes of the carotenoid biosynthesis pathway (FIG. 3) that are essential for the targeted host plant to accumulate carotenes and/or xanthophylls of interest. According to another aspect of the present disclosure, said methods can also be used to modify pre-existing carotenoid biosynthesis in order to up- or down-regulate accumulation of certain intermediates or products of interest. Furthermore, specific DNA molecules are provided which comprise nucleotide sequences carrying one or more expression cassettes capable of directing production of one or more enzymes characteristic for the carotenoid biosynthesis pathway selected from the group consisting of: phytoene synthase derived from plants, fungi or bacteria, phytoene desaturase derived from plants, fungi or bacteria, carotenoid reductase (phytoene desaturase) derived from plants or cyanobacteria, and lycopene cyclase derived from plants, fungi or bacteria.

According to some embodiments, the above expression cassette comprises one or more genes or cDNAs coding for plant, fungi or bacterial phytoene synthase, plant, fungi or bacterial phytoene desaturase, plant ζ-carotene desaturase, or plant, fungi or bacterial lycopene cyclase, each operably linked to a suitable constitutive, inducible or tissue-specific promoter allowing its expression in plant cells, seeds, tissues or whole plants. In some embodiments genes or cDNAs code for a plant phytoene synthase, bacterial phytoene desaturase or plant lycopene cyclase. A large, still increasing number of genes coding for phytoene synthase from plants and bacterium (WO 98/06862, WO 99/55889, U.S. Pat. No. 5,545,816; U.S. Pat. No. 5,705,624, U.S. Pat. No. 5,750,865) and, Crt I-type carotene desaturase (bacterial) and lycopene cyclase (plant and bacterial) have been isolated and are accessible from the databases. They are from various sources and they are all available for use in the methods of the present disclosure. In some embodiments the phytoene synthase coding sequence is from a plant. In some embodiments the phytoene synthase polynucleotide is a phytoene synthase 1 coding sequence derived from Zea mays. In some embodiments the phytoene synthase coding sequence encodes a phytoene synthase 1 polypeptide having at least 70%, 75%, 80%, 85%, 90%, 91%, 92%, 93%, 94%, 95%, 96%, 97%, 98% or 99% sequence identity to SEQ ID NO: 7. In some embodiments the phytoene synthase polypeptide has at least 70%, 75%, 80%, 85%, 90%, 91%, 92%, 93%, 94%, 95%, 96%, 97%, 98% or 99% sequence identity to the amino acid sequence of SEQ ID NO: 7. In some embodiments, the phytoene synthase polynucleotide is a polynucleotide encoding the Zea mays phytoene synthase 1 of SEQ ID NO: 7. In specific embodiments, the phytoene synthase polynucleotide comprises the nucleic acid sequence of SEQ ID NO: 1. In some embodiments, the phytoene synthase polynucleotide is a codon optimized polynucleotide encoding the Zea mays phytoene synthase 1. In specific embodiments, the phytoene synthase polynucleotide comprises the nucleic acid sequence of SEQ ID NO: 33, or SEQ ID NO: 35. In some embodiments the phytoene synthase polynucleotide is a phytoene synthase 3 coding sequence is from Zea mays. In some embodiments the phytoene synthase 3 coding sequence encodes a phytoene synthase 3 polypeptide having at least 70%, 75%, 80%, 85%, 90%, 91%, 92%, 93%, 94%, 95%, 96%, 97%, 98% or 99% sequence identity to SEQ ID NO: 14. In some embodiments the phytoene synthase 3 polypeptide has at least 70%, 75%, 80%, 85%, 90%, 91%, 92%, 93%, 94%, 95%, 96%, 97%, 98% or 99% sequence identity to the amino acid sequence of SEQ ID NO: 14. In some embodiments, the phytoene synthase 3 polynucleotide is a polynucleotide encoding the Zea mays phytoene synthase 3 of SEQ ID NO: 14. In specific embodiments, the phytoene synthase polynucleotide comprises the nucleic acid sequence of SEQ ID NO: 41. In some embodiments, the phytoene synthase polynucleotide is a codon optimized polynucleotide encoding the Zea mays phytoene synthase Y.

In some embodiments the carotenoid reductase coding sequence is from a bacterium. In some embodiments the carotenoid reductase coding sequence is from Erwinia uredovora. In some embodiments the carotenoid reductase coding sequence encodes a carotenoid reductase polypeptide having at least 70%, 75%, 80%, 85%, 90%, 91%, 92%, 93%, 94%, 95%, 96%, 97%, 98% or 99% sequence identity to SEQ ID NO: 8. In some embodiments the carotenoid reductase polypeptide has at least 70%, 75%, 80%, 85%, 90%, 91%, 92%, 93%, 94%, 95%, 96%, 97%, 98% or 99% sequence identity to the amino acid sequence of SEQ ID NO: 8. In some embodiments, the carotenoid reductase polynucleotide is a polynucleotide encoding the Erwinia uredovora (now Pantoea anantis) Crt I-type carotene desaturase of SEQ ID NO: 8. In some embodiments, the Crt I-type carotene desaturase polynucleotide comprises the nucleic acid sequence of SEQ ID NO: 2. In some embodiments, the carotenoid reductase polynucleotide is a polynucleotide encoding the Erwinia uredovora (now Pantoea anantis) Crt B carotene desaturase of SEQ ID NO: 47. In some embodiments, the Crt I-type carotene desaturase polynucleotide is maize codon optimized comprises the nucleic acid sequence of SEQ ID NO: 48.

### Methylerythritol Phosphate (MEP) Pathway

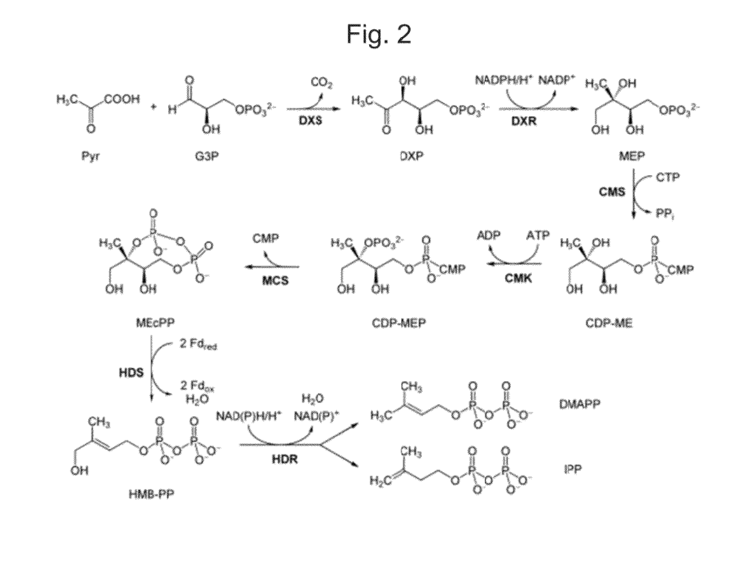

The biosynthesis of isopentenyl diphosphate (IPP) and dimethylallyl diphosphate (DMAPP), which are major building block in the formation of isoprenoids in many bacteria, green algae, and plant plastids are synthesized through the methylerythritol phosphate (MEP) pathway (FIG. 2) (for review see Rodriguez-Concepcion M, et. al. Plant Physiology (2002)130:1079-1089). The first steps leading from pyruvate (Pyr) and glyceraldehyde 3-phosphate (G3P) to 2-methylerythritol (ME) 2,4-cyclodiphosphate (CDP-ME) are well known, (M. Rohmer, Nat. Prod. Rep. (1999) 16, 565-573; W. Eisenreich, F. et. al. Trends Plant Sci. (2001) 6, 78-84]) the last steps leading to Isopentenyl diphosphate (IPP) and dimethylallyl diphosphate (DMAPP) and involving the gcpE gene encoding hydroxymethylbutenyl 4-diphosphate synthase (HDS) and the lytB gene encoding (E)-4-hydroxy-3-methyl-but-2-enyl diphosphate reductase have only more recently elucidated from bacteria. Cyclodiphosphate (CDP-ME) is the substrate of the hydroxymethylbutenyl 4-diphosphate synthase (HDS) converting CDP-ME into (E)-4-hydroxy-3-methylbut-2-enyl diphosphate (HMBPP). (E)-4-hydroxy-3-methyl-but-2-enyl diphosphate (HMBPP) reductase is the final step in the MEP pathway and is the branch point in the MEP pathway between IPP and DMAPP. Over expression of Arabidopsis thaliana D-1-deoxy-xylulose 5-phosphate synthase (DXS) Arabidopsis thaliana deoxyxylulose 5-phoshate reductoisomerase (DXR) has been shown to increase vitamin A levels in Arabidopsis thaliana (Carretero-Paulet, L. et al Plant Mol. Biol. 2006 November; 62(4-5):683-95).

In accordance with the subject disclosure, means and methods of transforming plant cells, seeds, tissues or whole plants are provided to produce transformants capable of expressing one or more enzymes of the methylerythritol phosphate pathway that are essential for the targeted host plant to accumulate carotenes and/or xanthophylls of interest. According to another aspect of the present disclosure, said methods can also be used to modify pre-existing carotenoid biosynthesis in order to up- or down-regulate accumulation of certain intermediates or products of interest. Specific DNA molecules are provided which comprise nucleotide sequences carrying one or more expression cassettes capable of directing production of one or more enzymes characteristic for the methylerythritol phosphate pathway selected from the group consisting of: D-1-deoxy-xylulose 5-phosphate synthase (DXS) derived from plants, fungi, or bacteria, deoxyxylulose 5-phoshate reductoisomerase (DXR), derived from plants, fungi or bacteria, hydroxymethylbutenyl diphosphate reductase (HDR) derived from plants, fungi or bacteria, hydroxymethylbutenyl 4-diphosphate synthase (HDS) derived from plants, fungi or bacteria, and (E)-4-hydroxy-3-methyl-but-2-enyl diphosphate reductase derived from plants, fungi or bacteria. In some embodiments, the D-1-deoxy-xylulose 5-phosphate synthase (DXS) coding sequence is derived from Arabidopsis thaliana. In some embodiments the D-1-deoxy-xylulose 5-phosphate synthase (DXS) coding sequence encodes a D-1-deoxy-xylulose 5-phosphate synthase (DXS) having at least 70%, 75%, 80%, 85%, 90%, 91%, 92%, 93%, 94%, 95%, 96%, 97%, 98% or 99% sequence identity to the amino acid sequence of SEQ ID NO: 10. In some embodiments the D-1-deoxy-xylulose 5-phosphate synthase (DXS) has at least 70%, 75%, 80%, 85%, 90%, 91%, 92%, 93%, 94%, 95%, 96%, 97%, 98% or 99% sequence identity to the amino acid sequence of SEQ ID NO: 10. In some embodiments the D-1-deoxy-xylulose 5-phosphate synthase (DXS) polynucleotide encodes the D-1-deoxy-xylulose 5-phosphate synthase (DXS) of SEQ ID NO: 10. In some embodiments the D-1-deoxy-xylulose 5-phosphate synthase (DXS) polynucleotide comprises the nucleic acid sequence of SEQ ID NO: 4. In some embodiments the DXS polynucleotide is a maize codon optimized polynucleotide encoding the D-1-deoxy-xylulose 5-phosphate synthase (DXS) of SEQ ID NO: 10. In some embodiments the codon optimized DXS polynucleotide comprises the nucleic acid sequence of SEQ ID NO: 31.

Tocotrienols and tocopherols comprise the vitamin E class of lipid soluble antioxidants in plants. These molecules are composed of a polar chromanol head group derived from the shikimate pathway bound to a C₂₀ isoprenoid-derived hydrocarbon tail. Tocotrienols and tocopherols differ only in their degree of unsaturation: the tocotrienol hydrocarbon chain contains three trans double bonds, whereas the tocopherol hydrocarbon chain is fully saturated. Within each class of vitamin E, four forms occur in plants α, β, γ and δ that differ in the numbers and positions of methyl residues on the chromanol head group. The a form of tocotrienols and tocopherols contains three methyl groups on the chromanol ring, the β and γ forms contain two methyl groups on the chromanol ring, but in different positions, and the δ form contains only one methyl group. Collectively, the eight forms of tocotrienols and tocopherols are referred to as tocochromanols.

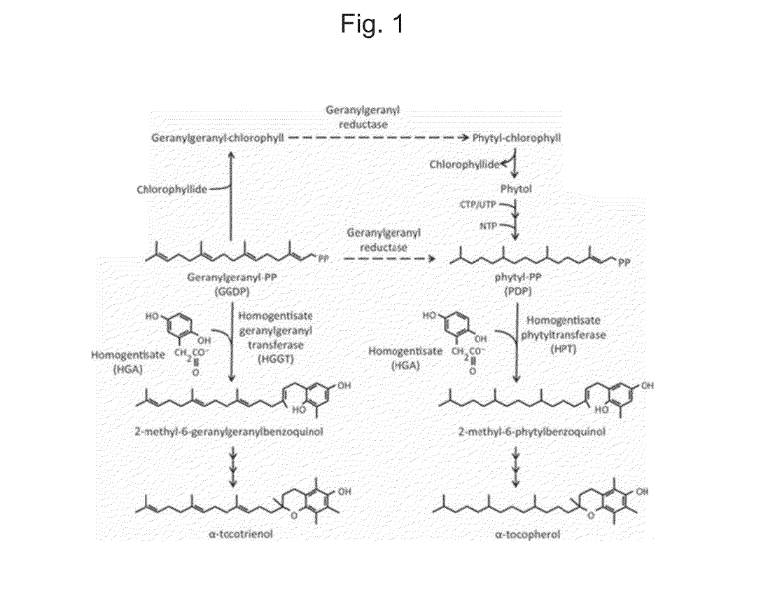

Tocotrienols and tocopherols are potent lipid soluble antioxidants. Although tocotrienols and tocopherols both function as antioxidants, these two classes of tocochromanols and the individual forms of each have distinct biological activities and physical properties. α-Tocopherol, for example, is generally considered to be the most nutritionally beneficial form of vitamin E because it is the most readily absorbed and retained by the body. Of the two classes of tocochromanols, tocopherols occur more widely in plants. Tocopherols, typically in the α form, are abundant in leaves of all plants, and are also enriched in seeds of most dicots and seed embryos of monocots (Cahoon E et. al., Nature Biotechnology (2003) 21:1082-1087). In contrast, the occurrence of tocotrienols is limited primarily to the seed endosperm of monocots and some dicots, including tobacco, grape and members of the Apiaceae family, where they are the major class of tocochromanols. The biosynthesis of tocochromanols occurs in plastids of plant cells. The initial step in tocochromanols biosynthesis (FIG. 1) is the condensation of homogentisate and phytyl diphosphate (PDP) to form 2-methyl-6-phytylbenzoquinol. This reaction is catalyzed by homogentisate phytyltransferase (HPT), which is encoded by VTE2 in Arabidopsis. For the synthesis of α-tocopherol, the initial HPT-catalyzed condensation reaction is followed by methylation, cyclization to form the chromanol head group and a second methylation. Tocotrienol biosynthesis is believed to involve reactions analogous to those associated with tocopherol biosynthesis (FIG. 1). The only difference is that the initial condensation reaction is presumed to use geranylgeranyl diphosphate (GGPP) instead of PDP, given the similarity in unsaturation between GGPP and the hydrocarbon chain of tocotrienols. The isolation of cDNAs for a structural variant of HPT from the monocots barley (Hordeum vulgare), wheat (Triticum aestivum) and rice (Oryza sativa), designated ‘homogentisate geranylgeranyl transferase’ (or ‘HGGT’) has been demonstrated (Cahoon E et. al., Nature Biotechnology (2003) 21:1082-1087; U.S. Pat. No. 7,154,029; U.S. Pat. No. 7,622,658; U.S. Pat. No. 8,269,076; WO 2003/082899). The transgenic expression of HGGT alone is sufficient to confer tocotrienol biosynthesis to plant organs and cells, such as Arabidopsis leaves and tobacco callus, which do not normally accumulate this form of vitamin E (Cahoon E et. al., Nature Biotechnology (2003) 21:1082-1087). Monocot HGGTs identified to date are related to HPTs, including those from monocot species, but share <50% amino acid sequence identity (Cahoon E et. al., Nature Biotechnology (2003) 21:1082-1087); and Arabidopsis (Venkatesh et. al., Planta (2006) 223:1134-44). Consistent with the restricted accumulation of tocotrienols in seed endosperm of monocots, expression of HGGT in barley was detected in seeds but was absent from leaves and roots (Cahoon E et. al., Nature Biotechnology (2003) 21:1082-1087; U.S. Pat. No. 7,154,029; U.S. Pat. No. 7,622,658; U.S. Pat. No. 8,269,076; WO 2003/082899). Monocot HGGT is most active with GGDP, but has activity with PDP, and can yield mixtures of tocotrienols and tocopherols in planta, the relative levels of which are likely to be dictated by the available pools of these substrates.

In accordance with the subject disclosure, means and methods of transforming plant cells, seeds, tissues or whole plants are provided to produce transformants capable of expressing one or more enzymes of the vitamin E biosynthesis pathway that increase carotenoid, particularly β-carotene, half-life and increase the bioaccessibility and bioavailability of carotenoids, particularly β carotene. According to another aspect of the present disclosure, said methods can also be used to modify pre-existing vitamin E biosynthesis in order to up- or down-regulate accumulation of certain intermediates or products of interest. Specific DNA molecules are provided which comprise nucleotide sequences carrying one or more expression cassettes capable of directing production of one or more enzymes characteristic for the vitamin E biosynthesis pathway selected from the group consisting of: by homogentisate phytyltransferase (HPT) derived from plants, fungi, or bacteria and homogentisate geranylgeranyl transferase (HGGT) derived from plants, fungi or bacteria. In some embodiments, the homogentisate geranylgeranyl transferase (HGGT) coding sequence is derived from Hordeum vulgare, Zea mays, Glycine max or Arabidopsis thaliana (U.S. Pat. No. 7,154,029; U.S. Pat. No. 7,622,658; U.S. Pat. No. 8,269,076; WO2003/082899). In some embodiments, the homogentisate geranylgeranyl transferase (HGGT) coding sequence is derived from Hordeum vulgare. In some embodiments the homogentisate geranylgeranyl transferase (HGGT) coding sequence encodes a homogentisate geranylgeranyl transferase (HGGT) having at least 70%, 75%, 80%, 85%, 90%, 91%, 92%, 93%, 94%, 95%, 96%, 97%, 98% or 99% sequence identity to the amino acid sequence of SEQ ID NO: 11. In some embodiments the homogentisate geranylgeranyl transferase (HGGT) has at least 70%, 75%, 80%, 85%, 90%, 91%, 92%, 93%, 94%, 95%, 96%, 97%, 98% or 99% sequence identity to the amino acid sequence of SEQ ID NO: 11. In some embodiments the homogentisate geranylgeranyl transferase (HGGT) polynucleotide encodes of homogentisate geranylgeranyl transferase (HGGT) polypeptide of SEQ ID NO: 11. In some embodiments the homogentisate geranylgeranyl transferase (HGGT) polynucleotide comprises the nucleic acid sequence of SEQ ID NO: 5.

### Carotenoid-Associated Proteins

In plant cells, carotenoids are located mainly in chloroplasts and chromoplasts. During fruit maturation and flower morphogenesis plastids are converted into chromoplasts. During this process, the thylakoid membranes disintegrate, chlorophyll and most of the components of the photosynthetic machinery disappear, and there is a massive accumulation of carotenoids in novel structures leading to the classification of chromoplasts as globular, crystalline, membranous, fibrillar and tubular. Fibrillar chromoplasts accumulate extremely high levels of protein in the fibril's external half-membrane. These proteins accumulate in parallel to carotenoid accumulation and chromoplast fibril formation during flower morphogenesis and fruit ripening. Collectively, these proteins have been termed carotenoid-associated proteins (CAP), because they are components of the carotenoid-protein complexes resolved from chromoplast fibrils. Carotenoid-associated protein (CAP) genes have been identified from a number of plants including: Pisum sativum (GenBank accession # AF043905); Arabidopsis thaliana (GenBank accession # AL021712); Brassica campestris (J. T. L. Ting et al. Plant J., 16 (1998), pp. 541-551); Cucumis sativus (M. Vishnevetsky et al. Plant J., 10 (1996), pp. 1111-1118); Nicotiana tabacum (GenBank accession # Y15489); Capsicum annuum (J. Deruere et al. Plant Cell, 6 (1994), pp. 119-133; GenBank accession # X97559.1); Solanum tuberosum (B. Gillet et al. Plant J., 16 (1998), pp. 257-262); Citrusunshiu (T. Moriguchi et al. Biochim. Biophys. Acta, 1442 (1998), pp. 334-338); and Synechocystis sp. (D90904). The Orange (Or) gene mutation (Lu S. et al., The Plant Cell, 18, 3594-3605 2006) is believed to act as a molecular switch to trigger the differentiation of non-colored plastids into chromoplasts. The overexpression of the Or gene from cauliflower in transgenic potatoes has been shown to lead to the increased accumulation of carotenoids and carotenoid sequestering structures (Lu S. et al., The Plant Cell, 18, 3594-3605 2006; Giuliano and Diretto, 2007).

In accordance with the subject disclosure, means and methods of transforming plant cells, seeds, tissues or whole plants are provided to produce transformants capable of expressing a carotenoid-associated protein (CAP) gene to increase the accumulation of carotenoids, particularly β-carotene. Specific DNA molecules are provided which comprise nucleotide sequences carrying one or more expression cassettes capable of directing production of one or more a carotenoid-associated protein (CAP). In some embodiments the carotenoid-associated protein (CAP) is derived from the carotenoid-associated protein (CAP) from Capsicum annuum (J. Deruere et al. Plant Cell, 6 (1994), pp. 119-133; GenBank accession # X97559.1). In some embodiments the carotenoid-associated protein (CAP) coding sequence encodes a carotenoid-associated protein (CAP) polypeptide having at least 70%, 75%, 80%, 85%, 90%, 91%, 92%, 93%, 94%, 95%, 96%, 97%, 98% or 99% sequence identity to the amino acid sequence of SEQ ID NO: 12. In some embodiments the carotenoid-associated protein (CAP) has at least 70%, 75%, 80%, 85%, 90%, 91%, 92%, 93%, 94%, 95%, 96%, 97%, 98% or 99% sequence identity to the amino acid sequence of SEQ ID NO: 12. In some embodiments the carotenoid-associated protein (CAP) polynucleotide encodes of carotenoid-associated protein (CAP) polypeptide of SEQ ID NO: 12. In some embodiments the carotenoid-associated protein (CAP) polynucleotide is a maize codon optimized polynucleotide encoding the carotenoid-associated protein (CAP) polypeptide of SEQ ID NO: 12. In some embodiments the maize codon optimized carotenoid-associated protein (CAP) polynucleotide comprises the nucleic acid sequence of SEQ ID NO: 36.

In accordance with the subject disclosure, means and methods of transforming plant cells, seeds, tissues or whole plants are provided to produce transformants capable of expressing an Orange (Or) gene to increase the accumulation of carotenoids, particularly β-carotene. Specific DNA molecules are provided which comprise nucleotide sequences carrying one or more expression cassettes capable of directing production of one or more orange (Or) protein. In some embodiments the orange (Or) protein is derived from orange (Or) gene (At5g61670) from Arabidopsis thaliana (GenBank accession # NM-203246). In some embodiments the orange (Or) protein coding sequence encodes an orange (Or) protein having at least 70%, 75%, 80%, 85%, 90%, 91%, 92%, 93%, 94%, 95%, 96%, 97%, 98% or 99% sequence identity to the amino acid sequence of SEQ ID NO: 13. In some embodiments the orange (Or) protein has at least 70%, 75%, 80%, 85%, 90%, 91%, 92%, 93%, 94%, 95%, 96%, 97%, 98% or 99% sequence identity to the amino acid sequence of SEQ ID NO: 13. In some embodiments the orange (Or) protein polynucleotide encodes the orange (Or) protein of SEQ ID NO: 13. In some embodiments the orange (Or) protein polynucleotide is a maize codon optimized polynucleotide encoding the orange (Or) polypeptide of SEQ ID NO: 13. In some embodiments the maize codon optimized orange (Or) protein polynucleotide comprises the nucleic acid sequence of SEQ ID NO: 38.

### Phytate Biosynthesis Pathway Silencing

Phytic acid in cereal grains and oilseeds is poorly digested and negatively affects nutrition. Phosphorus content of cereal grains and oilseeds is bound in phytic acid and therefore not available to for optimal phosphorus nutrition. In addition, phytic acid reduces the bioavailability of essential mineral cations, such as Fe³⁺, Zn²⁺ and Ca²⁺. Phytic acid also interacts with basic amino acids, seed proteins and enzymes in the digestive tract to form complexes that may reduce amino acid availability, protein digestibility and the activity of digestive enzymes.

In developing seeds, phytic acid is synthesized from glucose-6-phosphate, which is converted to myo-inositol 3-phosphate (Ins(3)P) by Ins(3)P synthase (MIPS).

Dephosphorylation of Ins(3)P produces myo-inositol. Stepwise phosphorylation of myo-inositol and Ins(3)P leads to phytic acid. Mutation and silencing of genes in this pathway also can produce low-phytic-acid, high-P, seed including but not limited to Ins(1,3,4,5,6)P₅2-kinase (IP2K) (U.S. Pat. No. 7,714,187); IPTK-5; inositol polyphosphate kinase (IPPK), Lpa2 (see U.S. Pat. Nos. 5,689,054 and 6,111,168); myo-inositol 1-phosphate synthase (MIPS), myo-inositol kinase (MIK, also known as CHOK or Lpa3) (US20080020123) and myo-inositol monophosphatase (IMP) (see WO 99/05298).

Low-phytic-acid (Ipa) mutants, which accumulate Pᵢ without a change in total phosphorus content, have been identified in all major crops (Raboy, V., Trends in Plant Science 6:458-62, 2001). Of the three known classes of maize Ipa mutants, Ipa1 mutants have the lowest phytate levels. The gene disrupted in maize Ipa1 mutants has recently been shown to be a multidrug resistance-associated protein (MRP) ATP-binding cassette (ABC) transporter (Shi, J. et al., Nature Biotechnology 25: 930-937 2007; U.S. Pat. No. 8,080,708; U.S. Pat. No. 7,511,198). Silencing expression of this transporter in an embryo-specific manner was shown to produce low-phytic-acid, high-Pᵢ transgenic maize seeds that germinate normally and do not show any significant reduction in seed dry weight. In some embodiments the low-phytic-acid (Ipa1) mutant is derived from the multidrug resistance-associated protein (MRP) ATP-binding cassette (ABC) transporter (Shi, J. et al., Nature Biotechnology 25: 930-937 2007; U.S. Pat. No. 8,080,708; U.S. Pat. No. 7,511,198). In some embodiments the low-phytic-acid (Ipa1) mutant polynucleotide has at least 60%, 65%, 70%, 75%, 80%, 85%, 90%, 91%, 92%, 93%, 94%, 95%, 96%, 97%, 98% or 99% sequence identity to the nucleic acid sequence of SEQ ID NO: 46. In some embodiments the low-phytic-acid (Ipa1) mutant polynucleotide comprises the nucleic acid sequence of SEQ ID NO: 46.

In some embodiments suppression may be used to inhibit the expression of one or more genes in the phytate biosynthesis pathway. See, for example, Broin et al. (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell et al. (1994) Proc. Natl. Acad. Sci. USA 91:3490-3496; Jorgensen et al. (1996) Plant Mol. Biol. 31:957-973; Johansen and Carrington (2001) Plant Physiol. 126:930-938; Broin et al. (2002) Plant Cell 14:1417-1432; Stoutjesdijk et al (2002) Plant Physiol. 129:1723-1731; Yu et al. (2003) Phytochemistry 63:753-763; and U.S. Pat. Nos. 5,034,323, 5,283,184, and 5,942,657; each of which are herein incorporated by reference. The efficiency of cosuppression may be increased by including a poly-dT region in the expression cassette at a position 3′ to the sense sequence and 5′ of the polyadenylation signal. See, U.S. Patent Publication No. 20020048814, herein incorporated by reference.

In some embodiments the DNA molecules further comprise at least one selectable marker gene or cDNA operably linked to a suitable constitutive, inducible or tissue-specific promoter. Examples of selectable marker genes include but are not limited to phosphomannose isomerase (PMI) and hygromycin phosphotransferase under the control of a constitutive promoter. In specific embodiments the phosphomannose isomerase polynucleotide comprises the nucleic acid sequence of SEQ ID NO: 3. Although the skilled person may select any available promoter functionally active in plant material, it is preferred in the design of appropriate expression cassettes according to the disclosure to operably link the respective nucleotide sequence encoding, carotenoid reductase (phytoene desaturase), lycopene cyclase, deoxy-xylulose phosphate synthase, and homogentisate geranylgeranyl transferase to tissue-specific or constitutive promoters. In some embodiments the nucleotide sequence encoding phytoene synthase is expressed under the control of a tissue-specific promoter to avoid interference with gibberellin-formation.

It is to be understood that the nucleotide sequence as a functional element of the DNA molecule according to the disclosure can comprise any combination of one or more of the above-mentioned genes or cDNAs. In some embodiment of the present disclosure, said nucleotide sequence comprises functional expression cassettes for phytoene synthase, phytoene desaturase, deoxy-xylulose phosphate synthase, and homogentisate geranylgeranyl transferase, which are stacked in the appropriate plasmid or vector system and are introduced into target plant material. In some embodiment of the present disclosure, the nucleotide sequence comprises at least one of the functional expression cassette, which can after incorporation into an appropriate plasmid or vector system be introduced into target plant material, either alone or together with at least one additional vector comprising at least one additional nucleotide sequence comprises at least one of the functional expression cassette.

The disclosure further provides plasmids or vector systems comprising one or more of the above DNA molecules or nucleotide sequences, which are derived from Agrobacterium tumefaciens.

The subject disclosure additionally provides transgenic plant cells, seeds, tissues and whole plants that display an improved nutritional quality and contain one or more of the above DNA molecules, plasmids or vectors, and/or that have been generated by use of the methods according to the present disclosure.

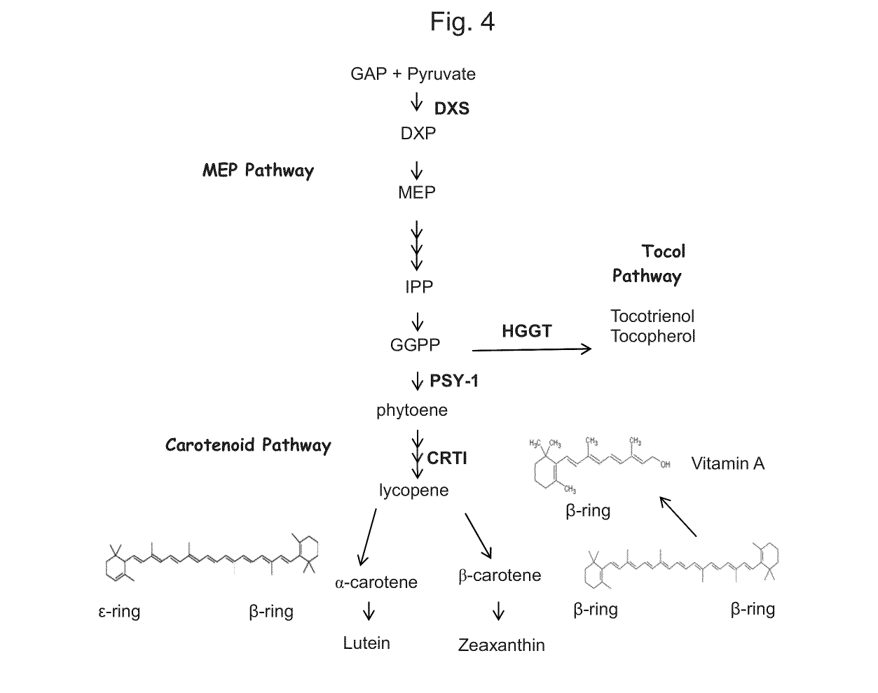

The current disclosure is based on the fact that the early intermediate geranylgeranyl diphosphate (GGPP) does not only serve for carotenogenesis but represents a branching point serving several different biosynthetic pathways (FIG. 4). It is therefore concluded that this precursor occurs in the plastids of all plant tissues, carotenoid-bearing or not, such as rice endosperm. The source of GGPP can thus be used to achieve the objects of the present disclosure, i.e. the introduction of the carotenoid biosynthetic pathway in part or as a whole, and/or the enhancement or acceleration of a pre-existing carotenoid biosynthetic pathway, and/or increasing carotenoid half-life, bioaccessibility and bioavailability.

The term “carotenoid-free” used throughout the specification to differentiate between certain target plant cells or tissues shall mean that the respective plant material not transformed according to the disclosure is known normally to be essentially free of carotenoids, as is the case for e.g. storage organs such as endosperm and the like. Carotenoid-free does not mean that those cells or tissues that accumulate carotenoids in almost undetectable amounts are excluded. The term shall define plant material having a carotenoid content of 0.001% w/w or lower.

In some embodiments of the present disclosure a higher plant phytoene synthase is operatively linked to a promoter conferring tissue-specific expression. This is unified on the same plasmid or vector with a bacterial (Crt-1-type) phytoene desaturase, the latter fused to a DNA sequence coding for a transit peptide and operatively linked to an endosperm preferred promoter allowing tissue specific expression. The transformation of plants with this construct in a suitable vector will direct the formation of lycopene in the tissue selected by the promoter controlling phytoene synthase, for example, in the endosperm of cereal seeds. This transformation alone can initiate carotenoid synthesis beyond lycopene formation towards downstream xanthophylls, such as lutein, zeaxanthin, antheraxanthin, violaxanthin, and neoxanthin in the endosperm. In addition the formation of α-carotene is observed. Thus, a carotenoid complement similar to the one present in green leaves is formed. A further advantage of using bacterial phytoene desaturase of the Crt I-type in the transformation is that said enzyme will be expressed also in leaf chloroplasts, thereby conferring resistance to bleaching herbicides targeting plant phytoene desaturase.

The plasmid or vector may carry the gene for a deoxy-xylulose phosphate synthase, and homogentisate geranylgeranyl transferase, equipped with a transit-sequence may be used. This is operatively linked to a promoter, preferably conferring the same tissue-specificity of expression as with phytoene synthase. The transformation of the plant with the expression cassette results in the complementing the seed target tissue with the full information for carrying out the carotenoid biosynthetic pathway to form β-carotene.

The genes used can be operatively equipped with a DNA sequence coding for a transit-sequence allowing plastid-import. This is done either by recombinant DNA technology or the transit-sequence is present in the plant cDNA in use. The transformation then allows carotenoid formation using a pool of the precursor geranylgeranyl-diphosphate localized in plastids. This central compound is neither a carotenoid nor does it represent a precursor that is solely devoted to carotenoid biosynthesis (see FIG. 4).

The plants should express the gene(s) introduced, and are preferably homozygous for expression thereof. Generally, the gene will be operably linked to a promoter functionally active in the targeted host cells of the particular plant. The expression should be at a level such that the characteristic desired from the gene is obtained. For example, the expression of the selectable marker gene should provide for, an appropriate selection of transformants yielded according to the methods of the present disclosure. Similarly, the expression of one or more genes of the carotenoid and xanthophyll biosynthetic pathway for enhanced nutritional quality should result in a plant having a relatively higher content of one or more of the pathway intermediates or products compared to that of the same species which is not subjected to the transformation method according to the present disclosure. On the other hand, it will generally be desired to limit the excessive expression of the gene or genes of interest in order to avoid significantly adversely affecting the normal physiology of the plant, i.e. to the extent that cultivation thereof becomes difficult.

The gene or genes encoding the enzyme or enzymes of interest can be used in expression cassettes for expression in the transformed plant tissues. To achieve the objects of the present disclosure, i.e., to introduce or complement the carotenoid biosynthetic pathway in a target plant of interest, the plant is transformed with at least one expression cassette comprising a transcriptional initiation region linked to a gene of interest.

The transcriptional initiation may be native or analogous to the host or foreign or heterologous to the host. By foreign is intended that the transcriptional initiation region is not found in the wild-type host into which the transcriptional initiation region is introduced. Of particular interest are those transcriptional initiation regions associated with storage proteins, such as zeins, kafirins, glutelin, patatin, napin, cruciferin, β-conglycinin, phaseolin, or the like.

The transcriptional cassette will include, in 5′-3′ direction of transcription, a transcriptional and translational initiation region, a DNA sequence of interest, and a transcriptional and translational termination region functional in plants. By “terminator” is intended sequences that are needed for termination of transcription: a regulatory region of DNA that causes RNA polymerase to disassociate from DNA, causing termination of transcription. The termination region may be native with the transcriptional initiation region, may be native with the DNA sequence of interest, or may be derived from other sources. Convenient termination regions are available from the Ti-plasmid of A. tumefaciens such as the octopine synthase and nopaline synthase termination regions (see also, Guerineau et al., 1991; Proudfoot, 1991; Sanfacon et al., 1991, Mogen et al., 1990; Munroe et al., 1990; Ballas et al., 1989; Joshi et al., 1987). In specific embodiments the terminator is from the Sorghum bicolor legumin coding sequence (US7,897,841). In specific embodiments the terminator is the Sorghum bicolor legumin 1 terminator (SB-LEG1 TERM). In specific embodiments the Sorghum bicolor legumin 1 terminator comprises the nucleic acid sequence of SEQ ID NO: 16. In specific embodiments the terminator is from the Sorghum bicolor gamma kafirin coding sequence. In specific embodiments the terminator is the Sorghum bicolor gamma kafirin terminator (SB-GKAF TERM). In specific embodiments the Sorghum bicolor gamma kafirin terminator comprises the nucleic acid sequence of SEQ ID NO: 19. In specific embodiments the terminator is from the Solanum tuberosum proteinase inhibitor II coding sequence (An et al., 1989, Plant Cell 1:115-122; Keil et al., 1986). In specific embodiments the terminator is the Solanum tuberosum proteinase inhibitor II terminator (PINII TERM) (An et al., 1989, Plant Cell 1:115-122; Keil et al., 1986). In specific embodiments the Solanum tuberosum proteinase inhibitor II terminator comprises the nucleic acid sequence of SEQ ID NO: 23. In specific embodiments the terminator is from the 27 kD gamma zein coding sequence of Zea mays (Reina et al., (1990) Nucleic Acids Res 18(21): 6426). In specific embodiments the Zea mays 27 kD gamma zein terminator (GZ-W64A TERM) comprises the nucleic acid sequence of SEQ ID NO: 25. In specific embodiments the terminator is N-(aminocarbonyl)-2-chlorobenzenesulfonamide inducible terminator (Inducible (IN) TERM) isolated from Zea mays (U.S. Pat. No. 5,364,780). In specific embodiments the Zea mays IN terminator (IN2-1 TERM) comprises the nucleic acid sequence of SEQ ID NO: 27. In specific embodiments the seed preferred terminator is from the Globulin 1 gene of Zea mays (Belanger F C et. al. (1989) Plant Physiol. 91:636-643). In specific embodiments the Globulin 1 terminator (GLB1 TERM) comprises the nucleic acid sequence of SEQ ID NO: 29. In specific embodiments the terminator is from the ubiquitin 1 coding sequence of Sorghum bicolor (isolated from Sb line: P898012). In specific embodiments the Sorghum bicolor ubiquitin terminator (SB UB1 TERM) comprises the nucleic acid sequence of SEQ ID NO: 43. In specific embodiments the terminator is from the actin coding sequence of Sorghum bicolor (U.S. Ser. No. 61/655,087). In specific embodiments the Sorghum bicolor actin terminator (SB ACTIN TERM) comprises the nucleic acid sequence of SEQ ID NO: 42. In specific embodiments the terminator is derived from the Zea mays 22 Kd zein mutant floury-2 (FL2) gene, having a “floury” phenotype. In specific embodiments the Zea mays FL2 terminator (FL2 TERM) comprises the nucleic acid sequence of SEQ ID NO: 39. In specific embodiments the terminator is from the Zea mays EAP1 (Early Abundant Protein 1) coding region coding sequence (U.S. Pat. No. 7,081,566; U.S. Pat. No. 7,321,031). In specific embodiments the EAP1 terminator (EAP1 TERM) comprises the nucleic acid sequence of SEQ ID NO: 45.

For the most part, the gene or genes of interest of the present disclosure will be targeted to plastids, such as chloroplasts, for expression. In this manner, where the gene of interest is not directly inserted into the plastid, the expression cassette will additionally contain a sequence encoding a transit peptide to direct the gene of interest to the plastid. Such transit peptides are known in the art (see, for example, Von Heijne et al., 1991; Clark et al., 1989; Della-Cioppa et al., 1987; Romer et al., 1993; and, Shah et al., 1986. Any carotenoid pathway genes useful in the disclosure can utilize native or heterologous transit peptides. In specific embodiments the transit peptide is from the ribulose-1,5-bisphosphate carboxylase small subunit coding sequence from Pisum sativum (Coruzzi, et al, J. Biol. Chem. 258:1399-1402, 1983). In specific embodiments the transit peptide is a Pisum sativum ribulose-1,5-bisphosphate carboxylase small subunit transit peptide (PS SSU TP) comprises the amino acid sequence of SEQ ID NO: 6. In specific embodiments the Pisum sativum ribulose-1,5-bisphosphate carboxylase small subunit transit peptide (PS SSU TP) is encoded by the nucleic acid sequence of SEQ ID NO: 17. In specific embodiments the transit peptide is a Coriandrum sativum delta-4-palmitoyl-ACP desaturase gene transit peptide (CS-DPAD TP) comprises the amino acid sequence of SEQ ID NO: 49. In specific embodiments the Coriandrum sativum delta-4-palmitoyl-ACP desaturase gene transit peptide (CS-DPAD TP) is encoded by a maize codon optimized polynucleotide having the nucleic acid sequence of SEQ ID NO: 50.

The construct can also include any other necessary regulators such as plant translational consensus sequences (Joshi, 1987), introns (Luehrsen and Walbot, 1991) and the like, operably linked to the nucleotide sequence of interest. Intron sequences within the gene desired to be introduced may increase its expression level by stabilizing the transcript and allowing its effective translocation out of the nucleus. Among the known such intron sequences are the introns of the plant ubiquitin gene (Cornejo, 1993). Furthermore, it has been observed that the same construct inserted at different loci on the genome can vary in the level of expression in plants. The effect is believed to be due at least in part to the position of the gene on the chromosome, i.e., individual isolates will have different expression levels (see, for example, Hoever et al., 1994). In some embodiments the intron is from the maize alcohol dehydrogenase 1 (adh1) gene (Mascarenhas D, et al. (1990) Plant Mol Biol 15: 913-920). In some embodiments the intron is the adh1 intron 6 comprising the nucleic acid sequence of SEQ ID NO: 30.

Further regulatory DNA sequences that may be used for the construction of expression cassettes include, for example, sequences that are capable of regulating the transcription of an associated DNA sequence in plant tissues in the sense of induction or repression.

It may be beneficial to include 5′ leader sequences in the expression cassette construct. Such leader sequences can act to enhance translation. Translation leaders are known in the art and include: picornavirus leaders, for example, EMCV leader (Encephalomyocarditis 5′ noncoding region; Elroy-Stein et al., 1989); potyvirus leaders, for example, TEV leader (Tobacco Etch Virus; Allisson et al., 1986); and human immunoglobulin heavy-chain binding protein (BiP, Macejak and Sarnow, 1991); untranslated leader from the coat protein mRNA of alfalfa mosaic virus (AMV RNA 4; Jobling and Gehrke 1987); tobacco mosaic virus leader (TMV; Gallie et al., 1989); and maize chlorotic mottle virus leader (MCMV; Lommel et al., 1991; see also, Della-Cioppa et al., 1987).

Depending upon where the DNA sequence of interest is to be expressed, it may be desirable to synthesize the sequence with plant preferred codons, or alternatively with chloroplast preferred codons. The plant preferred codons may be determined from the codons of highest frequency in the proteins expressed in the largest amount in the particular plant species of interest (see, EP-A 0 359 472; EP-A 0 386 962; WO 91/16432; Perlak et al., 1991; and Murray et al., 1989). In this manner, the nucleotide sequences can be optimized for expression in any plant. It is recognized that all or any part of the gene sequence may be optimized or synthetic. That is, synthetic or partially optimized sequences may also be used. For the construction of chloroplast preferred genes (see U.S. Pat. No. 5,545,817).

In preparing the transcription cassette, the various DNA fragments may be manipulated, so as to provide for the DNA sequences in the proper orientation and, as appropriate in the proper reading frame. Towards this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resection, ligation, or the like may be employed, where insertions, deletions or substitutions, e.g. transitions and transversions, may be involved.

The expression cassette carrying the gene of interest is placed into an expression vector by standard methods. The selection of an appropriate expression vector will depend upon the method of introducing the expression vector into host cells. A typical expression vector contains: prokaryotic DNA elements coding for a bacterial replication origin and an antibiotic resistance gene to provide for the growth and selection of the expression vector in the bacterial host; a cloning site for insertion of an exogenous DNA sequence, which in this context would code for one or more specific enzymes of the carotenoid biosynthetic pathway; eukaryotic DNA elements that control initiation of transcription of the exogenous gene, such as a promoter; and DNA elements that control the processing of transcripts, such as a transcription termination/poly-adenylation sequence. It also can contain such sequences as are needed for the eventual integration of the vector into the chromosome.

In some embodiments, the expression vector also contains a gene encoding a selection marker such as, e.g. hygromycin phosphotransferase (van den Elzen et al., 1985), which is functionally linked to a promoter. Additional examples of genes that confer antibiotic resistance and are thus suitable as selectable markers include those coding for neomycin phosphotransferase kanamycin resistance (Velten et al., 1984); the kanamycin resistance (NPT II) gene derived from Tn5 (Bevan et al., 1983); the PAT gene described in Thompson et al., (1987); and chloramphenicol acetyltransferase. For a general description of plant expression vectors and selectable marker genes suitable according to the present disclosure, see Gruber et al., (1993).

A number of promoters can be used in the practice of the embodiments. The promoters can be selected based on the desired outcome. The nucleic acids can be combined with constitutive, tissue-preferred, inducible or other promoters for expression in the host organism. Suitable constitutive promoters for use in a plant host cell include, for example, the core promoter of the Rsyn7 promoter and other constitutive promoters disclosed in WO 1999/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142 and 6,177,611.

Depending on the desired outcome, it may be beneficial to express the gene from an inducible promoter. Chemical-regulated promoters can be used to modulate the expression of a gene in a plant through the application of an exogenous chemical regulator. Depending upon the objective, the promoter may be a chemical-inducible promoter, where application of the chemical induces gene expression or a chemical-repressible promoter, where application of the chemical represses gene expression. Chemical-inducible promoters are known in the art and include, but are not limited to, the maize In2-1 and In2-2 promoter (U.S. Pat. No. 5,364,780), which are activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena, et al., (1991) Proc. Natl. Acad. Sci USA 88:10421-10425 and McNellis, et al., (1998) Plant J. 14(2):247-257) and tetracycline-inducible and tetracycline-repressible promoters (see, for example, Gatz, et al., (1991) Mol. Gen. Genet. 227:229-237 and U.S. Pat. Nos. 5,814,618 and 5,789,156), herein incorporated by reference.

Tissue-preferred promoters can be utilized to target polypeptide expression within a particular plant tissue. By “promoter” is intended a regulatory region of DNA usually comprising a TATA box capable of directing RNA polymerase II to initiate RNA synthesis at the appropriate transcription initiation site for a particular coding sequence. A promoter can additionally comprise other recognition sequences generally positioned upstream or 5′ to the TATA box, referred to as upstream promoter elements, which influence the transcription initiation rate. It is recognized that having identified the nucleotide sequences for the promoter region disclosed herein, it is within the state of the art to isolate and identify further regulatory elements in the 5′ untranslated region upstream from the particular promoter region identified herein. Thus the promoter region disclosed herein is generally further defined by comprising upstream regulatory elements such as those responsible for tissue and temporal expression of the coding sequence, enhancers and the like. Tissue-preferred promoters include those discussed in Yamamoto, et al., (1997) Plant J. 12(2)255-265; Kawamata, et al., (1997) Plant Cell Physiol. 38(7):792-803; Hansen, et al., (1997) Mol. Gen. Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol. Biol. 23(6):1129-1138; Matsuoka, et al., (1993) Proc Natl. Acad. Sci. USA 90(20):9586-9590 and Guevara-Garcia, et al., (1993) Plant J. 4(3):495-505. Such promoters can be modified, if necessary, for weak expression.

Leaf-preferred promoters are known in the art. See, for example, Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138 and Matsuoka, et al., (1993) Proc. Natl. Acad. Sci. USA 90(20):9586-9590.

Root-preferred or root-specific promoters are known and can be selected from the many available from the literature or isolated de novo from various compatible species. See, for example, Hire, et al., (1992) Plant Mol. Biol. 20(2):207-218 (soybean root-specific glutamine synthetase gene); Keller and Baumgartner, (1991) Plant Cell 3(10):1051-1061 (root-specific control element in the GRP 1.8 gene of French bean); Sanger, et al., (1990) Plant Mol. Biol. 14(3):433-443 (root-specific promoter of the mannopine synthase (MAS) gene of Agrobacterium tumefaciens) and Miao, et al., (1991) Plant Cell 3(1):11-22 (full-length cDNA clone encoding cytosolic glutamine synthetase (GS), which is expressed in roots and root nodules of soybean). See also, Bogusz, et al., (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi, (1991) describe their analysis of the promoters of the highly expressed roIC and roID root-inducing genes of Agrobacterium rhizogenes (see, Plant Science (Limerick) 79(1):69-76). They concluded that enhancer and tissue-preferred DNA determinants are dissociated in those promoters. Teeri, et al., (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see, EMBO J. 8(2):343-350). The TR1′ gene fused to nptII (neomycin phosphotransferase II) showed similar characteristics. Additional root-preferred promoters include the VfENOD-GRP3 gene promoter (Kuster, et al., (1995) Plant Mol. Biol. 29(4):759-772) and roIB promoter (Capana, et al., (1994) Plant Mol. Biol. 25(4):681-691. See also, U.S. Pat. Nos. 5,837,876; 5,750,386; 5,633,363; 5,459,252; 5,401,836; 5,110,732 and 5,023,179.

“Seed-preferred” promoters include both “seed-specific” promoters (those promoters active during seed development such as promoters of seed storage proteins) as well as “seed-germinating” promoters (those promoters active during seed germination). See, Thompson, et al., (1989) BioEssays 10:108, herein incorporated by reference. By “seed-preferred” is intended favored spatial expression in the seed, including at least one of embryo, kernel, pericarp, endosperm, nucellus, aleurone, pedicel, and the like. Such seed-preferred promoters include, but are not limited to, Cim1 (cytokinin-induced message); cZ19B1 (maize 19 kDa zein); and milps (myo-inositol-1-phosphate synthase) (see, U.S. Pat. No. 6,225,529, herein incorporated by reference). Gamma-zein is an endosperm-specific promoter and Glb-1 is an embryo specific. By “embryo-preferred” is intended favored spatial expression in the embryo of the seed. For dicots, seed-specific promoters include, but are not limited to, Kunitz trypsin inhibitor 3 (KTi3) (Jofuku and Goldberg, (1989) Plant Cell 1:1079-1093), bean β-phaseolin, napin, β-conglycinin, glycinin 1, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also, WO 2000/12733, where seed-preferred promoters from end1 and end2 genes are disclosed; herein incorporated by reference. A promoter that has “preferred” expression in a particular tissue is expressed in that tissue to a greater degree than in at least one other plant tissue. Some tissue-preferred promoters show expression almost exclusively in the particular tissue. In specific embodiments the seed preferred promoter is from the alpha kafirin coding sequence of Sorghum bicolor, which is intended to preferentially express in the endosperm (de Freitas et al., (1994) Mol. Gen. Genet., 245 (2):177-186). In specific embodiments the promoter is a Sorghum bicolor alpha kafirin A1 promoter (de Freitas et al., (1994) Mol. Gen. Genet., 245 (2):177-186). In specific embodiments the Sorghum bicolor alpha kafirin A1 promoter (SB-AKAF A1 PRO) comprises the polynucleotide of SEQ ID NO: 26. In specific embodiments the promoter is a Sorghum bicolor alpha kafirin B1 promoter (SB-AKAF B1 PRO) (de Freitas et al., (1994) Mol. Gen. Genet., 245 (2):177-186). In specific embodiments the Sorghum bicolor alpha kafirin B1 promoter (SB-AKAF B1 PRO) comprises the polynucleotide of SEQ ID NO: 15. In specific embodiments the seed preferred promoter is from the beta kafirin coding sequence of Sorghum bicolor, which is intended to preferentially express in the endosperm (Reddy et al., Journal of Plant Biochemistry and Biotechnology, 10(2): 101-106, July 2001). In specific embodiments the Sorghum bicolor beta kafirin promoter (SB-BKAF PRO) comprises the nucleic acid sequence of SEQ ID NO: 18. In specific embodiments the seed preferred promoter is from the gamma kafirin coding sequence of Sorghum bicolor, which is intended to preferentially express in the endosperm. In specific embodiments the Sorghum bicolor gamma kafirin promoter (SB-GKAF PRO) comprises the nucleic acid sequence of SEQ ID NO: 37. In specific embodiments the seed preferred promoter is from the delta kafirin coding sequence of Sorghum bicolor, which is intended to preferentially express in the endosperm (US7,847,160). In specific embodiments the seed preferred promoter is from the 27 kD gamma zein coding sequence of Zea mays (Reina et al., (1990) Nucleic Acids Res 18(21): 6426). In specific embodiments the Zea mays 27 kD gamma zein promoter (GZ-W64A PRO) comprises the nucleic acid sequence of SEQ ID NO: 24. In specific embodiments the seed preferred promoter is from the Globulin 1 gene of Zea mays (Belanger F C et. al. (1989) Plant Physiol. 91:636-643). In specific embodiments the Zea mays Globulin 1 promoter (GLB1 PRO) comprises the nucleic acid sequence of SEQ ID NO: 28. In specific embodiments the seed preferred promoter is from the Sorghum bicolor legumin 1 gene. In specific embodiments the legumin 1 promoter (SB-LEG1 PRO) comprises the nucleotide sequence of SEQ ID NO: 32. In specific embodiments the promoter is derived from the Zea mays 22 Kd zein mutant floury-2 (FL2) gene, having a “floury” phenotype. In specific embodiments the Zea mays FL2 promoter (FL2 PRO) comprises the nucleic acid sequence of SEQ ID NO: 40. In specific embodiments the promoter is from the Oleosin 1 coding sequence of Sorghum bicolor (U.S. Pat. No. 7,700,836). In specific embodiments the Sorghum bicolor Oleosin promoter (OLE PRO) comprises the nucleic acid sequence of SEQ ID NO: 44. In specific embodiments the promoter is from the ubiquitin coding sequence of Zea mays (Christensen et al., 1992, PMB 18: 675-689). In specific embodiments the Zea mays promoter (ZmUBI PRO) comprises the nucleic acid sequence of SEQ ID NO: 20.

Where low level expression is desired, weak promoters will be used. Generally, the term “weak promoter” as used herein refers to a promoter that drives expression of a coding sequence at a low level. By low level expression at levels of about 1/1000 transcripts to about 1/100,000 transcripts to about 1/500,000 transcripts is intended. Alternatively, it is recognized that the term “weak promoters” also encompasses promoters that drive expression in only a few cells and not in others to give a total low level of expression. Where a promoter drives expression at unacceptably high levels, portions of the promoter sequence can be deleted or modified to decrease expression levels.

Such weak constitutive promoters include, for example the core promoter of the Rsyn7 promoter (WO 1999/43838 and U.S. Pat. No. 6,072,050), the core 35S CaMV promoter, and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142 and 6,177,611, herein incorporated by reference.

The above list of promoters is not meant to be limiting. Any appropriate promoter can be used in the embodiments.

The plant cells, seeds, tissues and whole plants contemplated in the context of the present disclosure may be obtained by any of several methods. Those skilled in the art will appreciate that the choice of method might depend on the type of plant, i.e. monocot or dicot, targeted for transformation. Such methods generally include direct gene transfer, chemically-induced gene transfer, electroporation, microinjection (Crossway et al., 1986; Neuhaus et al., 1987), Agrobacterium-mediated gene transfer, ballistic particle acceleration using, for example, devices available from Agracetus, Inc, Madison, Wis., and DuPont, Inc., Wilmington, Del. (see, for example, Sanford et al., U.S. Pat. No. 4,945,050; and Mc Cabe et al., 1988), and the like.

One method for obtaining the present transformed plants or parts thereof is direct gene transfer in which plant cells are cultured or otherwise grown under suitable conditions in the presence of DNA oligonucleotides comprising the nucleotide sequence desired to be introduced into the plant or part thereof. The donor DNA source is typically a plasmid or other suitable vector containing the desired gene or genes. For convenience, reference is made herein to plasmids, with the understanding that other suitable vectors containing the desired gene or genes are also contemplated.

Any suitable plant tissue which takes up the plasmid may be treated by direct gene transfer. Such plant tissue includes, for example, reproductive structures at an early stage of development, particularly prior to meiosis, and especially 1-2 weeks pre-meiosis. Generally, the pre-meiotic reproductive organs are bathed in plasmid solution, such as, for example, by injecting plasmid solution directly into the plant at or near the reproductive organs. The plants are then self-pollinated, or cross-pollinated with pollen from another plant treated in the same manner. The plasmid solution typically contains about 10-50 μg DNA in about 0.1-10 ml per floral structure, but more or less than this may be used depending on the size of the particular floral structure. The solvent is typically sterile water, saline, or buffered saline, or a conventional plant medium. If desired, the plasmid solution may also contain agents to chemically induce or enhance plasmid uptake, such as, for example, PEG, Ca²⁺ or the like.

Following exposure of the reproductive organs to the plasmid, the floral structure is grown to maturity and the seeds are harvested. Depending on the plasmid marker, selection of the transformed plants with the marker gene is made by germination or growth of the plants in a marker-sensitive, or preferably a marker-resistant medium. For example, seeds obtained from plants treated with plasmids having the kanamycin resistance gene will remain green, whereas those without this marker gene are albino. Presence of the desired gene transcription of mRNA therefrom and expression of the peptide can further be demonstrated by conventional Southern, northern, and western blotting techniques.

In another method suitable to carry out the present disclosure, plant protoplasts are treated to induce uptake of the plasmid. Protoplast preparation is well-known in the art and typically involves digestion of plant cells with cellulase and other enzymes for a sufficient period of time to remove the cell wall. Typically, the protoplasts are separated from the digestion mixture by sieving and washing. The protoplasts are then suspended in an appropriate medium, such as, for example, medium F, CC medium, etc., typically at 10⁴-10⁷ cells/ml. To this suspension is then added the plasmid solution described above and an inducer such as polyethylene glycol, Ca²⁺, Sendai virus or the like. Alternatively, the plasmids may be encapsulated in liposomes. The solution of plasmids and protoplasts are then incubated for a suitable period of time, typically about 1 hour at about 25° C. In some instances, it may be desirable to heat shock the mixture by briefly heating to about 45° C., e.g. for 2-5 minutes, and rapidly cooling to the incubation temperature. The treated protoplasts are then cloned and selected for expression of the desired gene or genes, e.g. by expression of the marker gene and conventional blotting techniques. Whole plants are then regenerated from the clones in a conventional manner.

Another method suitable for transforming target cells involves the use of Agrobacterium. In this method, Agrobacterium containing the plasmid with the desired gene or gene cassettes is used to infect plant cells and insert the plasmid into the genome of the target cells. The cells expressing the desired gene are then selected and cloned as described above. For example, one method for introduction of a gene of interest into a target tissue, e.g., a tuber, root, grain or legume, by means of a plasmid, e.g. an Ri plasmid and an Agrobacterium, e.g. A. rhizogenes or A. tumefaciens, is to utilize a small recombinant plasmid suitable for cloning in Escherichia coli, into which a fragment of T-DNA has been spliced. This recombinant plasmid is cleaved open at a site within the T-DNA. A piece of “passenger” DNA is spliced into this opening. The passenger DNA consists of the gene or genes of this disclosure which are to be incorporated into the plant DNA as well as a selectable marker, e.g., a gene for resistance to an antibiotic. This plasmid is then recloned into a larger plasmid and then introduced into an Agrobacterium strain carrying an unmodified Ri plasmid. During growth of the bacteria, a rare double-recombination will sometimes take place resulting in bacteria whose T-DNA harbors an insert: the passenger DNA. Such bacteria are identified and selected by their survival on media containing the antibiotic. These bacteria are used to insert their T-DNA (modified with passenger DNA) into a plant genome. This procedure utilizing A. rhizogenes or A. tumefaciens give rise to transformed plant cells that can be regenerated into healthy, viable plants (see, for example, Zhao et.al. Methods In Molecular Biology Volume: 343, Issue: 15, 2006, Pages: 233-244; Zhao, Z.-Y. et. al. Plant Molecular Biology 2000, 44: 789-798); U.S. Pat. No. 6,369,298; U.S. Pat. No. 8,143,484; Carvalho C. H. S., Genetics and Molecular Biology: 27:259-169, 2004).

Another suitable approach is bombarding the cells with microprojectiles that are coated with the transforming DNA (Wang et al., 1988), or are accelerated through a DNA containing solution in the direction of the cells to be transformed by a pressure impact thereby being finely dispersed into a fog with the solution as a result of the pressure impact (EP-A 0 434 616).

Microprojectile bombardment has been advanced as an effective transformation technique for cells, including cells of plants. In Sanford et al., (1987), it was reported that microprojectile bombardment was effective to deliver nucleic acid into the cytoplasm of plant cells of Allium cepa (onion). Christou et al., (1988) reported the stable transformation of soybean callus with a kanamycin resistance gene via microprojectile bombardment. The same authors reported penetration at approximately 0.1% to 5% of cells and found observable levels of NPTII enzyme activity and resistance in the transformed calli of up to 400 mg/l of kanamycin. McCabe et al., (1988) report the stable transformation of Glycine max (soybean) using microprojectile bombardment. McCabe et al. further report the recovery of a transformed R1 plant from an RO chimeric plant (also see, Weissinger et al., 1988; Datta et al., 1990 (rice); Klein et al., 1988a (maize); Klein et al., 1988b (maize); Fromm et al., 1990; and Gordon-Kamm et al., 1990 (maize).

Alternatively, a plant plastid can be transformed directly. Stable transformation of chloroplasts has been reported in higher plants, see, for example, SVAB et al., (1990); SVAB and Maliga, (1993); Staub and Maliga, (1993). The method relies on particle gun delivery of DNA containing a selectable marker and targeting of the DNA to the plastid genome through homologous recombination. In such methods, plastid gene expression can be accomplished by use of a plastid gene promoter or by trans-activation of a silent plastid-borne transgene positioned for expression from a selective promoter sequence such as recognized by T7 RNA polymerase. The silent plastid gene is activated by expression of the specific RNA polymerase from a nuclear expression construct and targeting the polymerase to the plastid by use of a transit peptide. Tissue-specific expression may be obtained in such a method by use of a nuclear-encoded and plastid-directed specific RNA polymerase expressed from a suitable plant tissue-specific promoter. Such a system has been reported in McBride et al., (1994).

The list of possible transformation methods given above by way of example is not claimed to be complete and is not intended to limit the subject of the disclosure in any way.

The present disclosure therefore also comprises transgenic plant material, selected from the group consisting of protoplasts, cells, calli, tissues, organs, seeds, embryos, ovules, zygotes, etc. and especially, whole plants, that has been transformed by means of the method according to the disclosure and comprises the recombinant DNA of the disclosure in expressible form, and processes for the production of the said transgenic plant material.

Positive transformants are regenerated into plants following procedures well-known in the art (see, for example, McCormick et al., 1986). These plants may then be grown, and either pollinated with the same transformed strainer or different strains before the progeny can be evaluated for the presence of the desired properties and/or the extent to which the desired properties are expressed and the resulting hybrid having the desired phenotypic characteristic identified. A first evaluation may include, for example, the level of bacterial/fungal resistance of the transformed plants. Two or more generations may be grown to ensure that the subject phenotypic characteristic is stably maintained and inherited and then seeds harvested to ensure the desired phenotype or other property has been achieved.

Further comprised within the scope of the present disclosure are transgenic plants, in particular transgenic fertile plants transformed by means of the method of the disclosure and their asexual and/or sexual progeny, which still display the new and desirable property or properties due to the transformation of the mother plant.

The term ‘progeny’ is understood to embrace both, “asexually” and “sexually” generated progeny of transgenic plants. This definition is also meant to include all mutants and variants obtainable by means of known processes, such as for example cell fusion or mutant selection and which still exhibit the characteristic properties of the initial transformed plant, together with all crossing and fusion products of the transformed plant material.

Parts of plants, such as for example flowers, stems, fruits, leaves, roots originating in transgenic plants or their progeny previously transformed by means of the method of the disclosure and therefore consisting at least in part of transgenic cells, are also an object of the present disclosure.

Further comprised within the scope of the present disclosure are methods for increasing total carotenoid levels, increasing carotenoid half-life, increasing carotenoid bioavailability, increasing iron and zinc bioavailability, increasing carotenoid bioaccessibility, increasing grain digestibility, and any combination thereof in a transgenic plant cell; a transgenic plant or progeny thereof; or transgenic plant part, particularly in the seed or grain thereof. In some embodiments the method comprises expressing, in a transgenic plant cell; a transgenic plant or progeny thereof; or transgenic plant part, particularly in the seed or grain thereof, one or more enzymes of the vitamin E biosynthesis pathway. In particular embodiments the method comprises expressing a homogentisate geranylgeranyl transferase. In some embodiments the method comprises expressing in a transgenic plant cell; a transgenic plant or progeny thereof; or transgenic plant part, particularly in the seed or grain thereof, at least one enzyme in the carotenoid biosynthesis pathway. In particular embodiments the method comprises expressing a phytoene synthase and/or a phytoene desaturase. In some embodiments the method comprises expressing, in a transgenic plant cell; a transgenic plant or progeny thereof; or transgenic plant part, particularly in the seed or grain thereof, one or more enzymes of the methylerythritol phosphate pathway. In particular embodiments the method comprises expressing an D-1-deoxy-xylulose 5-phosphate synthase. In some embodiments the method comprises expressing, in a transgenic plant cell; a transgenic plant or progeny thereof; or transgenic plant part, particularly in the seed or grain thereof, one or more carotenoid-associated protein. In some embodiments the method comprises expressing, in a transgenic plant cell; a transgenic plant or progeny thereof; or transgenic plant part, particularly in the seed or grain thereof, one or more Orange (Or) mutant gene. In some embodiments the method comprises suppression of at least one or more genes in the phytate biosynthesis pathway. In some embodiments the method comprises suppression of low-phytic-acid (Ipa) mutants. In particular embodiments the method comprises expressing one or more enzymes of the vitamin E biosynthesis pathway; one or more enzymes in the carotenoid biosynthesis pathway; one or more enzymes of the methylerythritol phosphate pathway; one or more carotenoid-associated protein; one or more Orange (Or) mutant gene; suppression of one or more genes in in the phytate biosynthesis pathway; any and all combinations thereof.

In some embodiments the total carotenoid level in the transgenic plant is increased at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, 500% or more compared to a comparable non-transgenic plant for a carotenoid biosynthesis enzyme, a carotenoid accumulation protein, a methylerythritol phosphate biosynthesis enzyme, and/or a tocopherol/tocotrienol biosynthesis enzyme.

In some embodiments the beta-carotene level in the transgenic plant is increased at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, 500% or more compared to a comparable non-transgenic plant for a carotenoid biosynthesis enzyme, a carotenoid accumulation protein, a methylerythritol phosphate biosynthesis enzyme, and/or a tocopherol/tocotrienol biosynthesis enzyme.

In some embodiments the beta-carotene level in the transgenic plant is increased at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300% or more compared to comparable transgenic plant having a transgene only for a carotenoid biosynthesis enzyme.

In some embodiments the total carotenoid half-life is increased 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, 500%, or more compared to a comparable plant not having a tocopherol/tocotrienol biosynthesis enzyme.

In some embodiments the beta-carotene half-life is increased 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, 500% or more compared to a comparable plant not having a tocopherol/tocotrienol biosynthesis enzyme.

In some embodiments the total carotenoid bioavailability is increased 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, or more compared to a comparable plant not having a tocopherol/tocotrienol biosynthesis enzyme.

In some embodiments the beta-carotene bioavailability is increased 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, 500% or more compared to a comparable plant not having a tocopherol/tocotrienol biosynthesis enzyme.

The following examples are illustrative but not limiting of the present disclosure.

### EXAMPLES

### Example 1

### Construction of pABS168 Vector

Molecular biology techniques well known in the arts were used to assemble the transgene cassettes for pABS168 and other transgene cassettes in subsequent examples. The SB-AKAF B1 promoter of Sorghum bicolor (SEQ ID NO: 15) was operationally linked to the phytoene synthasel (ZM-PSY1) gene from maize (SEQ ID NO: 1) and the terminator region (SB-LEG1 TERM) from the legumin gene of Sorghum bicolor (SEQ ID NO: 16). Similarly, a nucleic acid molecule encoding a fusion of the small subunit gene chloroplast transit peptide (PEA SSU TP) coding sequence of pea (SEQ ID NO: 17) with the coding sequence of the crtl gene of Erwinia uredovora (SEQ ID NO: 2) was inserted between the SB-BKAF promoter (SEQ ID NO: 18) and SB-GKAF terminator (SEQ ID NO: 19). Both transgene cassettes were stacked using Gateway® recombinational cloning into a binary destination vector comprising a plant selectable marker; the promoter, 5′ untranslated region, and first intron of the ubiquitin) gene of Z. mays (the promoter (SEQ ID NO: 20); 5′UTR (SEQ ID NO: 21); first intron (SEQ ID NO: 22) linked to phosphomannose isomerase coding sequence (pmi) from E. coli (SEQ ID NO: 3) and the PINII terminator (SEQ ID NO: 23).

### Example 2

### Construction of pABS188 Vector

The pABS188 vector contains all the transgene cassettes found in pABS168 with the exception the phytoene synthasel gene is operably linked to the 27K gamma zein promoter (GZ-W64A-PRO) from maize (SEQ ID NO: 24), stacked with a co-suppression cassette comprising the GLB1 promoter (SEQ ID NO: 28) and GLB1 terminator (SEQ ID NO: 29) from the Globulin 1 gene of Zea mays (Belanger F C et. al. (1989) Plant Physiol. 91:636-643) operably linked to two copies of a truncated version of low phytic acid 1 (LPA-1) coding sequence (SEQ ID NO: 46) from Sorghum bicolor arranged in reverse orientation relative to each other and separated by a nucleic acid sequence for the Zea mays ADH1 intron 6 (SEQ ID NO: 30).

### Example 3

### Construction of pABS198 Vector

pABS198 vector contains all the transgene cassettes found in pABS168, with the addition of a transgene cassette comprising the GZ-W64A PRO promoter (SEQ ID NO: 24) and GZ-W64A TERM terminator (SEQ ID NO: 25) from the gamma zein gene of Zea mays operably linked to the Arabidopsis thaliana DXS (D-1-deoxy-xylulose 5-phosphate synthase) coding sequence (SEQ ID NO: 4).

### Example 4

### Construction of pABS203 Vector

pABS203 was constructed using the same transgene cassettes of pABS198, plus a cassette comprising the SB-AKAF A1 promoter (SEQ ID NO: 26) and the IN2-1 terminator (SEQ ID NO: 27) from Zea mays operably linked to the homogentisate geranylgeranyl transferase (HGGT) coding sequence from Hordeum vulgare (SEQ ID NO: 5).

### Example 5

### Plant Material and Transformation

Sorghum genotype TX430 grown in a greenhouse was used for transformation. Agrobacterium-mediated sorghum transformation was conducted using immature embryo explants isolated from sorghum TX430 following the protocol described by Zhao et al., (Zhao, Z. Y. Methods In Molecular Biology Volume: 343, issue: 15, 2006, Pages: 233-244; Zhao, Z. Y. et. al, Plant Molecular Biology 2000, 44: 789-798). Agrobacterium strain LBA4404 carrying JT super-binary vectors with the phosphornannose isomerase (pmi) gene as selection marker was used for all the transformations.

### Example 6

### Sorghum Seeds Harvest Procedure and Storage Condition

Panicles were collected from sorghum plants grown in the greenhouse 40 days after pollination (DAP) and air dried in room temperature for additional two weeks before threshing. The threshed seeds (including T1, T2 and T3 seeds) were stored in-80° C. freezer until immediately before conducting any experiments.

### Example 7

### HPLC Analysis of Carotenoids

All extractions procedures were completed under low light to minimize the potential for photo-oxidative reactions and carotenoids were analyzed by HPLC with UV detection.

Basically, sorghum seeds were ground with Geno grinder and the weight of the ground material was recorded and then extracted with 5 mL cold acetone and then with 2 mL methyl tert-butyl ether. The extract was dried under a stream of nitrogen, resolubilized in 1:1 methanol:ethyl acetate, then analyzed by HPLC and UV detection. The HPLC method is a modification of methods described previously (Paine J A, et al. Nature Biotechnology 2005, 23(4): 482-487) using a Waters YMC Carotenoid 5 μm (4.6×250 mm) column or equivalent and a Waters 2487 UV Detector or equivalent was used for detecting carotenoids in the testcross progenies. Samples were loaded into an amber glass auto sampler and carotenoids were detected at 450 nm at a flow rate of 2 ml min of 75% methanol and 25% Methyl-tert-Butyl-Ether (MTBE), with each run taking 25 min. Quantification of compounds was accomplished by standard regression with external standards. The carotenoid content in sorghum grains were reported as μg/gm wet weight.

### Example 8

### HPLC Analysis of Tocopherol and Tocotrienol Content

The tocotrienols and tocopherols were determined as described by Dolde et al (J Am Oil Chem Soc (2011) 88:1367-1372). Briefly, sorghum seeds were ground with Geno grinder and the weight of the ground material was recorded and then extracted with 2 mL of hexane under reduced lighting. Tocochromanols were separated by using a Waters HPLC Alliance 2695 (Milford, Mass., USA) with a 3μ NH₂ 100A, 150 mm×3.0 mm column or equivalent and detected by fluorescence (Waters 2475 or equivalent) with EXA=292 nm and EMA=335 nm. An external calibration curve of 0.05, 0.1, 0.2, 0.5, 1.0, 2.5 and 5 ppm of each tocotrienol and tocopherol was used for quantification. The tocotrienol and tocopherol contents in sorghum grains were expressed as μg/g dry weight.

### Example 9

### Oxygen Induced Degradation of Carotenoids and Beta-Carotene in ABS168

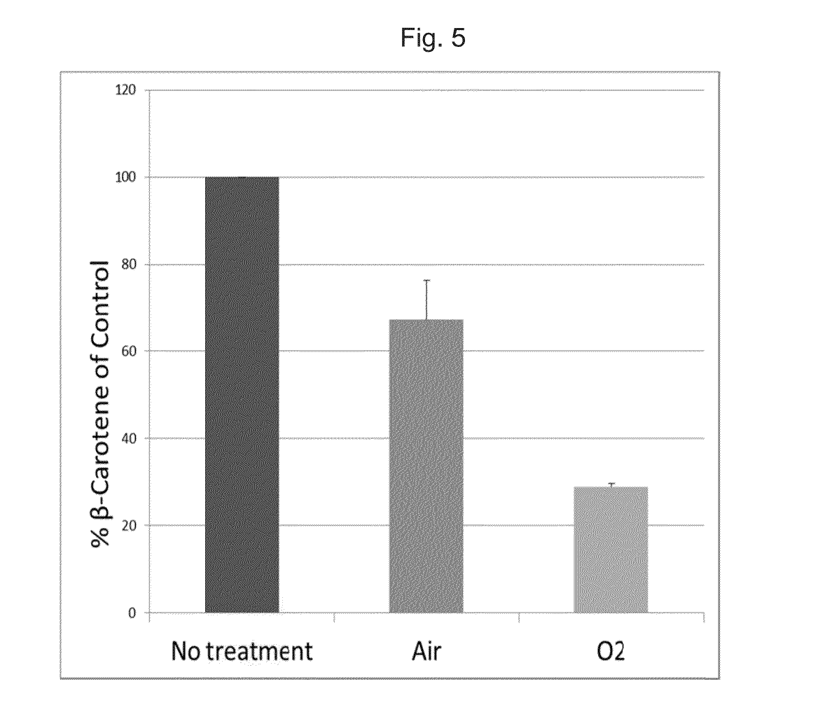

To test if oxidization is the main factor that causes beta-carotene degradation, ABS168 seeds from T2 plants were taken out from −80° C. and then either left in the air or in a sealed container that was purged with pure oxygen once a day (˜100% O₂) for 4 weeks. After four-week treatments, the β-carotene levels were determined by HPLC and the percentages of β-carotene retained in the seeds were calculated using the seeds from −80° C. as control. As demonstrated in FIG. 5, about 30% beta-carotene degraded after 4-week storage in the air. Beta-carotene degradation increased to 70% after 4-week storage in the pure oxygen. The results indicate that oxidization is the major factor that contributes to beta-carotene degradation.

### Example 10

### Select ABS 203 Events

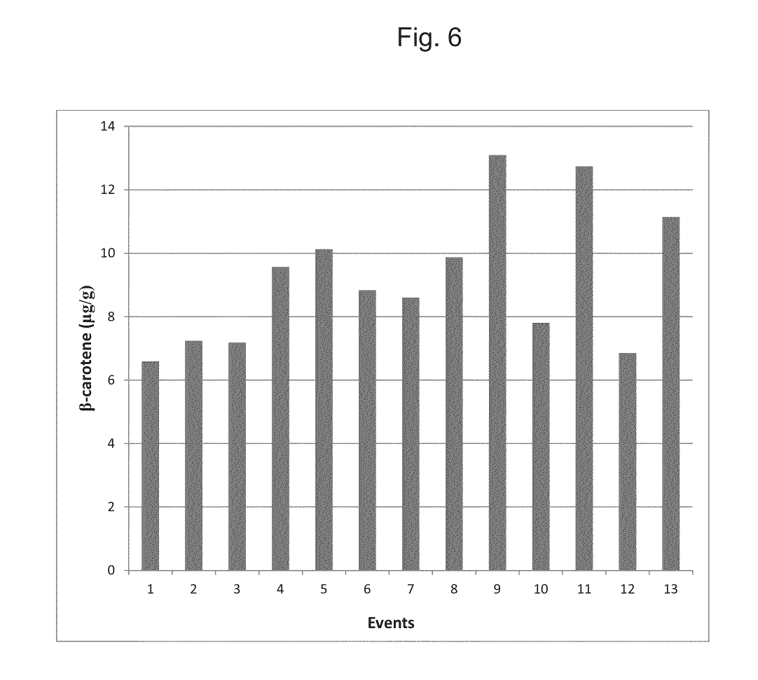

Twenty one ABS203 (PHP51136) independent single copy events were identified by PCR and QPCR from a total of 32 events generated by Agrobacterium-mediated sorghum transformation. 13 of these events which contained the highest carotenoid levels (especially beta-carotene) in the grain (T1 seeds) were selected for further generation selection. The homozygous T1 plants were identified by PCR and homozygous T2 seeds were harvested from the T1 homozygous plants and stored in −80° C. for further analysis. FIG. 6 shows the β carotene levels in seeds from T2 sorghum plants for the 13 ABS203 events.

### Example 11

### Determination of the Correlation Between β-Carotene and γ-Tocopherol

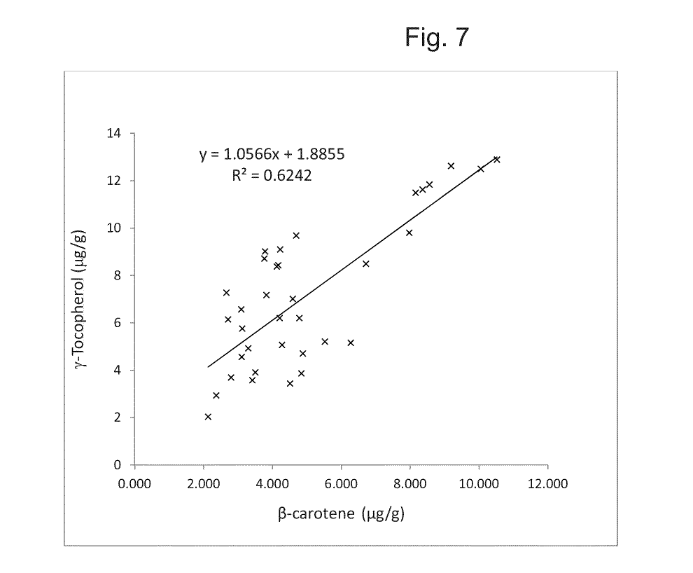

Both β-carotene and γ-Tocopherol levels of the 35 ABS203 T1 plants (two plants from each of the 18 events from Example 9, except one event where only one plant was produced) were analyzed by HPLC. The relationships between β-carotene and γ-tocopherol levels were determined according to the correlation coefficient. As shown in FIG. 7, a significant correlation (R²=0.6242) was observed between β-carotene and γ-tocopherol (and total tocochromanols, data not shown) among these 35 plants, indicating that the antioxidant function of vitamin E may increase the stability of β-carotene.

### Example 12

### Reduction of Oxygen-Induced β-Carotene Degradation by Tocotrienols and Tocopherols Expression

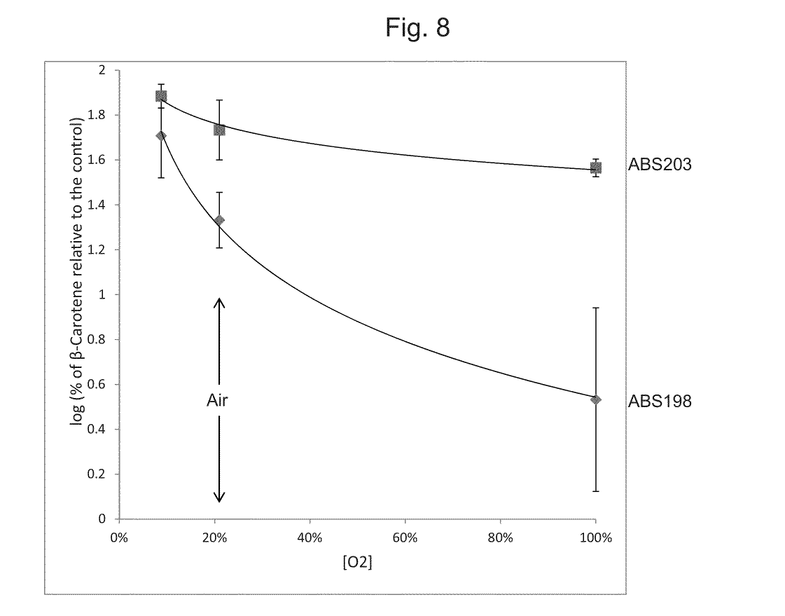

Transgenic sorghums (ABS203 and ABS198) were treated with different levels of oxygen for four weeks in room temperature. The different levels of oxygen were achieved either by leaving sorghum grains in the air (21% O₂), or in a sealed container that purged with pure oxygen once a day (100% O₂) or continually subjected to a vacuum pump which provides 60% vacuum power (9% O₂). After four-week treatments, the β-carotene levels were determined by HPLC and the percentages of β-carotene retained in the seeds were calculated. As demonstrated in FIG. 8, β-carotene degradation was increased with the increase oxygen level for both ABS203 and ABS198. The degradation rate of β-carotene in ABS203 (with HGGT) was much lower than ABS198 (without HGGT) strongly suggesting that oxidation is one of the main sources for β-carotene degradation, and the antioxidant function of tocotrienols and tocopherols plays important role in preventing β-carotene degradation and enhancing β-carotene stability under ambient storage condition.

### Example 13

### The Stability of β-Carotene is Highly Improved with HGGT Expression

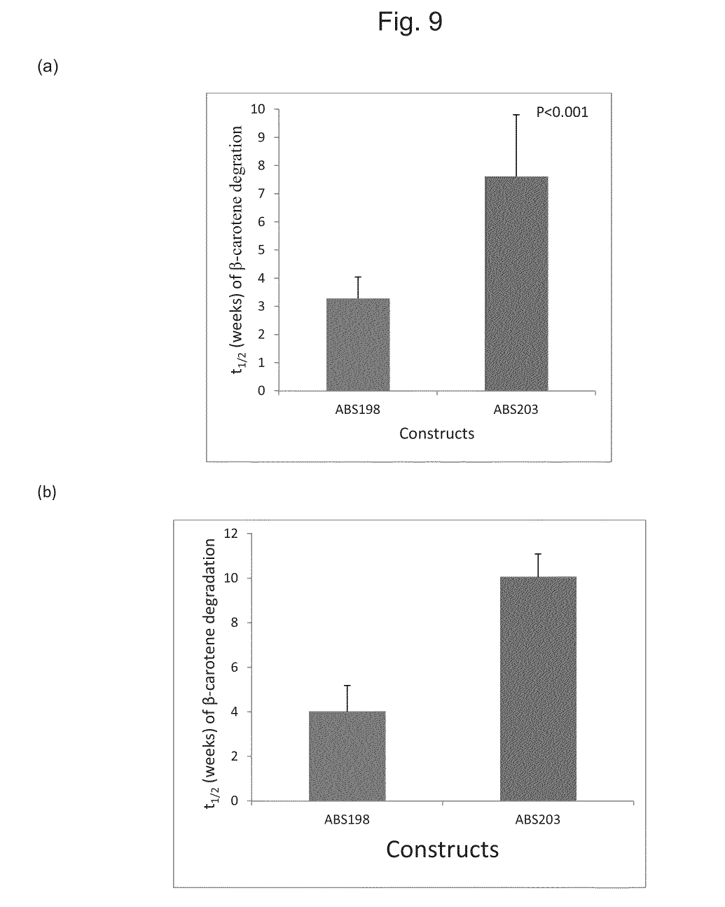

Sorghum seeds (ABS203 and ABS198) stored in −80° C. were left in room temperature for different time intervals (0, 2, 4 and 8 weeks, 4 and 6 months) and then stored back into −80° C. before HPLC analysis. Consistent with previously studies (Henry L K et. al. (1998) JAOCS Vol. 75, no. 7: 823-829; MInguez-Mosquera' M I et. al., (1994) J. Agric. Food Chem. 42:1551-1554; Alcides Oliveira R G et. al., (2010) African Journal of Food Science, Vol. 4(4):148-155; Tsimidon M et. al., (1993). J. Food Sci. 45:2890-2898; Goulson M J et. al. (1999) JOURNAL OF FOOD SCIENCE 64, No. 6:996-999; Ipek U, (2005) Biochemistry 40:621-624; Lavelli, V. (2006) IUFoST World Congress 13th World Congress of Food Science & Technology; Chen B H et. al. (1994) J. Agric. Food Chem., 42:2391-2397; Athanasia M. (2010) Drying Technology, 28:752-761), the degradation of beta-carotene in sorghum grains follows the first order kinetic order as well. The first order rate constants (k) of beta-carotene degradation were determined by plotting In[beta-carotene content] versus time. Therefore, the half-life times (t_{1/2}) of beta-carotene, the amount of time required for 50% degradation of its initial level, can be calculated according to the equation t_{1/2}=In2/k. It was determined that the half-life of β-carotene in ABS203 is about 8 weeks and the half-life of β-carotene in ABS198 is about 4 weeks, which means the stability of β-carotene is doubled with the coexpression of HGGT in sorghum endosperm (FIG. 9 panel a). The half-life (t_{1/2}) of β-carotene was also determined using ABS203 and ABS198 homozygous seeds as described above and similar results were obtained (FIG. 9 panel b)

### Example 14

### PSY1 Protein and Beta-Carotene Accumulation During Seeds Maturation

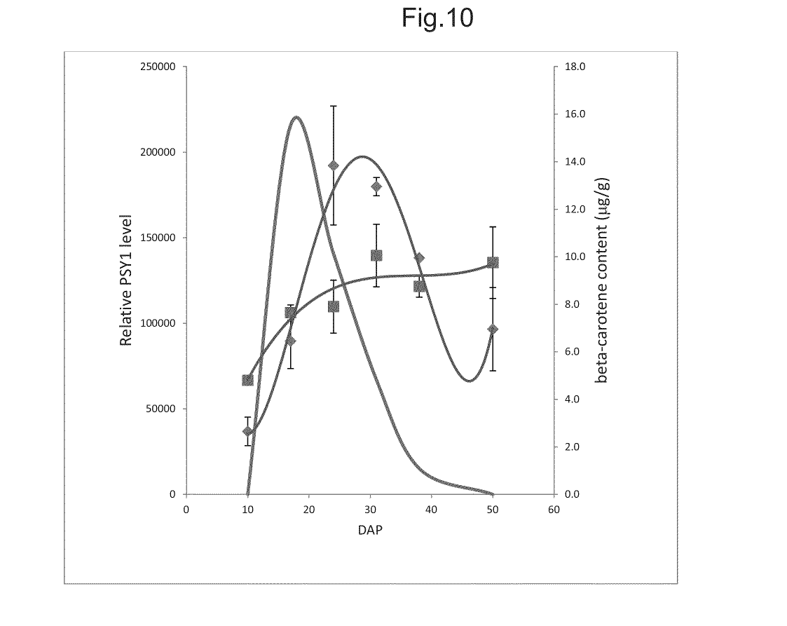

Immature seeds from T3 ABS203 plants at different seed development stages (10, 17, 24, 31, 38 DAP and mature) were collected and lyophilized. PSY1 protein accumulation was determined by Mass Spectrometry and as shown in FIG. 10, PSY1 accumulated to the highest level at milky stage (17, 21 DAP) and sharply declined to the undetected level at mature stage. The carotenoid levels at same development stages during seed maturation were determined by HPLC for both T3 ABS203 plants and T2 ABS198 plants. As shown in FIG. 10, Beta-carotene accumulated to the highest level after 30 DAP in both ABS203 and 198. However, beta-carotene level sharply declined in ABS198 after 30 DAP, but kept quite consistent in ABS203 until maturity.

### Example 15

### Seed Germination Experiment and Seed Weight Analysis

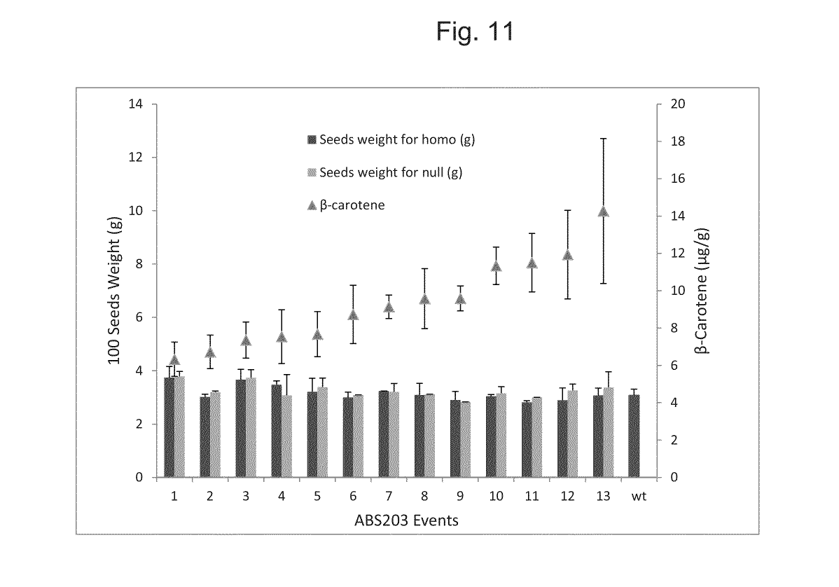

Seed germination was tested for the 13 top ABS203 events containing different level of carotenoids in greenhouse (FIG. 11). 30 seeds for each event were sown in the flat and well watered. Sorghum seedlings were counted after 10 days germination. 100% germination was observed for all of 13 events. Mature seeds were harvested at 40 DAP and air dried for two weeks at room temperature and the 100-Seed weight of each sample was determined (FIG. 11). No significant weight change was observed for all of 13 events.

### Example 16

### Carotenoid Bioavailability

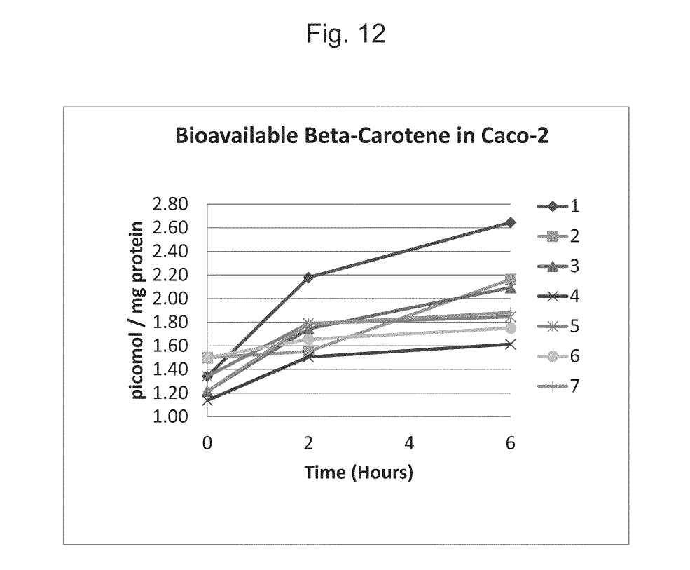

Seven samples were submitted to Purdue University for pro-vitamin A bioavailability analysis through Caco-2 cell. Among these 7 samples, 4 are ABS188 transgenic material with Carotenoids at 37.6 ug/g, 24.7 ug/g, 13.4 ug/g and 12.8 ug/g (Samples 1, 2, 3, and 4); two samples are null control with Carotenoids at 5.4 ug/g, 4/7 ug/g (Samples 5 and 6) and one non-transgenic control with Carotenoids at 5.5 ug/g (Sample 7). Through cooking (porridge preparation), in vitro digestion and micellerization, bioaccessibility and Caco-2 uptake; the Carotenoids and carotene from these 7 samples were measured (Liu, C.-S. et. al. J. Agric. Food Chem. 2004, 52, 4330-4337). Sample-1 showed the greatest beta carotene bioavailability levels in the Caco-2 system (FIG. 12).

### Example 17

### Construction of pABS210 Vector

The vector pABS210 encoding DXS (MO)+PSY1+CRTI was constructed using the same approaches as described above. The pABS210 vector comprises transgene cassettes GZ-W64A PRO/AT-DXS(MO)/GZ-W64A TERM//SB-AKAF B1 PRO (ALT1)/ZM-PSY1 (ALT1)/SB-LEG1 TERM//SB-BKAF PRO/PEA SSU TP::CRT I (EU)/SB-GKAF TERM. AT-DXS(MO) is a maize codon optimized version Arabidopsis thaliana DXS (D-1-deoxy-xylulose 5-phosphate synthase) gene having the nucleotide sequence of SEQ ID NO: 31.

### Example 18

### Construction Of pABS211 Vector

The vector pABS211 encoding DXS (MO)+PSY1+CRTI was constructed using the same approaches as described above. The pABS211 vector comprises transgene cassettes: SB-LEG1 PRO/AT-DXS(MO)/SB-LEG1 TERM//SB-AKAF B1 PRO (ALT1)/ZM-PSY1 (ALT1)/SB-LEG1 TERM//SB-BKAF PRO/PEA SSU TP::CRT I (EU)/SB-GKAF TERM. The DXS (MO) gene is operably linked to the Sorghum bicolor legumin 1 promoter (SB-LEG1 PRO) having the nucleotide sequence of SEQ ID NO: 32.

### Example 19

### Construction of pABS213 Vector

The vector pABS213 encoding PSY1(V1)+CRTI was constructed using the same approaches as described above. The pABS213 vector comprises transgene cassettes: CAMV35S ENH (−343-90)/GZ-W64A PRO/ZM-PSY1 (ALT1) (V1)/SB-UBI TERM//SB-BKAF PRO/PEA SSU TP::CRT I/SB-GKAF TERM (MOD1). PSY1(V1) is a maize codon optimized version of the Zea mays phytoene synthase 1 gene having the nucleotide sequence of SEQ ID NO: 33 operably linked to the GZ-W64A PRO promoter operably linked to a CAMV35S enhancer region (CAMV35S ENH) having a nucleotide sequence of SEQ ID NO: 34 and operably linked to the Sorghum bicolor ubiquitin terminator (SB-UBI TERM) having a nucleotide sequence of SEQ ID NO: 43.

### Example 20

### Construction of pABS214 Vector

The vector pABS214 encoding PSY1(V2)+CRTI was constructed using the same approaches as described above. The pABS213 vector comprises transgene cassettes: CAMV35S ENH (−343-90)/GZ-W64A PRO/ZM-PSY1 (ALT1) (V2)/SB-UBI TERM//SB-BKAF PRO/PEA SSU TP::CRT I/SB-GKAF TERM (MOD1). PSY1(V2) is a maize codon optimized version of the Zea mays phytoene synthase 1 gene having the nucleotide sequence of SEQ ID NO: 35.

### Example 21

### Construction of pABS220 Vector

The vector pABS220 encoding PSY1(V2)+CRTI was constructed using the same approaches as described above. The pABS213 vector comprises transgene cassettes: GZ-W64A PRO/CA-CAP (GENOMIC)/GZ-W64A TERM//CAMV35S ENH (−343-90)/SB-AKAF B1 PRO (ALT1)/ZM-PSY1 (ALT1) (V2)/GZ-W64A TERM//SB-BKAF PRO/PEA SSU TP::CRT I (EU)/SB-GKAF TERM. CA-CAP (GENOMIC) is a maize codon optimized Capsicum annum Carotenoid-Associated Protein (CAP) gene (fibrillin) having a nucleotide sequence of SEQ ID NO: 36.

### Example 22

### Construction of pABS219 Vector

The vector pABS219 encoding HV-HGGT+ZM-PSY(V1) was constructed using the same approaches as described above. The pABS213 vector comprises transgene cassettes: SB-GKAF PRO/HV-HGGT/SB-ACTIN TERM/GZ-W64A TERM//CAMV35S ENH (−343-90)/GZ-W64A PRO/ZM-PSY1 (ALT1)(V1)/SB-UBI TERM. HV-HGGT is operably linked to the Sorghum bicolor gamma kafirin promoter (SB-GKAF PRO) having a nucleotide sequence of SEQ ID NO: 37 and the Sorghum bicolor actin terminator (SB-ACTIN TERM) having a nucleotide sequence of SEQ ID NO: 42.

### Example 23

### Construction of pABS221 Vector

The vector pABS221 encoding HGGT+PSY3+PSY1(V1)+CRTI was constructed using the same approaches as described above. The pABS221 vector comprises transgene cassettes: SB-GKAF PRO/HV-HGGT/SB-ACTIN TERM//FL2 PRO (ALT1)/ZM-PSY3/FL2 TERM (ALT1)//CAMV35S ENH (−343-90)/SB-AKAF B1 PRO/ZM-PSY1 (ALT1)(V1)/SB-UBI TERM//SB-BKAF PRO/PEA SSU TP:CRT I/SB-GKAF TERM. ZM-PSY3 is a Zea mays phytoene synthase 3 having a nucleotide sequence of SEQ ID NO: 41 operably linked to the Zea mays FL2 promoter (FL2 PRO) having the nucleotide sequence of SEQ ID NO: 40.

### Example 24

### Construction of pABS223 Vector

The vector pABS223 encoding HGGT+CAP+PSY(V2)+CRTI was constructed using the same approaches as described above. The pABS223 vector comprises transgene cassettes: SB-GKAF PRO/HV-HGGT/SB-ACTIN TERM//GZ-W64A PRO/CA-CAP/GZ-W64A TERM//CAMV35S ENH (−343-90)/SB-AKAF B1 PRO (ALT1)/ZM-PSY1 (ALT1) (V2)/SB-UBI TERM//SB-BKAF PRO/PEA SSU TP::CRT I/SB-GKAF TERM.

### Example 25

### Construction of pABS224 Vector

The vector pABS224 encoding HGGT+PSY(V2)+CRTI was constructed using the same approaches as described above. The pABS224 vector comprises transgene cassettes: SB-GKAF PRO/HV-HGGT/SB-ACTIN TERM//CAMV35S ENH (−343-90)/GZ-W64A PRO/ZM-PSY1 (ALT1) (V2)/SB-UBI TERM//SB-BKAF PRO/PEA SSU TP::CRT I (EU)/SB-GKAF TERM.

### Example 26

### Construction of pABS226 Vector

The vector pABS226 encoding OR(MO)+PSY1(V2)+CRTI was constructed using the same approaches as described above. The pABS226 vector comprises transgene cassettes: FL2 PRO (ALT1)/AT-OR (MO)/FL2 TERM (ALT1)//CAMV35S ENH (−343-90)/SB-AKAF B1 PRO (ALT1)/ZM-PSY1 (ALT1) (V2)/SB-UBI TERM//SB-BKAF PRO/PEA SSU TP::CRT I (EU)/SB-GKAF TERM. AT-OR (MO) is a maize codon optimized version of the Arabidopsis thaliana orange protein gene having the nucleotide sequence of SEQ ID NO: 38 operably linked to FL2 PRO (ALT1) promoter having a nucleotide sequence of SEQ ID NO: 40 and the FL2 TERM having a nucleotide sequence of SEQ ID NO: 39.

### Example 27

### Construction of pABS227 Vector

The vector pABS227 encoding OR(MO)+PSY1(V2)+CRTI+HGGT was constructed using the same approaches as described above. The pABS227 vector comprises transgene cassettes: FL2 PRO (ALT1)/AT-OR (MO)/FL2 TERM (ALT1)//CAMV35S ENH (−343-90)/SB-AKAF B1 PRO (ALT1)/ZM-PSY1 (ALT1) (V2)/SB-UBI TERM//SB-BKAF PRO/PEA SSU TP::CRT I (EU)/SB-GKAF TERM//SB-GKAF PRO/HV-HGGT/SB-ACTIN TERM.

### Example 28

### Construction of pABS228 Vector

The vector pABS228 encoding OR(MO)+PSY1(V2)+CRTI+HGGT was constructed using the same approaches as described above. The pABS228 vector comprises transgene cassettes: FL2 PRO (ALT1)/AT-OR (MO)/FL2 TERM (ALT1)//CAMV35S ENH (−343-90)/SB-AKAF B1 PRO (ALT1)/ZM-PSY1 (ALT1) (V2)/SB-UBI TERM//SB-BKAF PRO/PEA SSU TP::CRT I (EU)/SB-GKAF TERM//SB-GKAF PRO/HV-HGGT/SB-ACTIN TERM//GZ-W64A PRO/CA-CAP (GENOMIC) (MO)/GZ-W64A TERM.

### Example 29

### Effect of PSY1 Driven by a Promoter with the 35S Enhancer on β-Carotene Accumulation

Transgenic sorghum plants transformed with the vectors pABS220 (Example 21) and pABS221 (Example 23) in which PSY1 was driven by the SB-KAFA B1 promoter with CAMV35S enhancer and vectors pABS210 (Example 17) and pABS211 (Example 18) without the enhancer were generated and β-carotene levels were measured, as described above, in their T1 seeds. Table 1 shows that beta-carotene accumulated at least 3 fold higher in the transgenic sorghum with the 35S enhancer (ABS220 and ABS221) compared with the transgenic sorghum without 35S enhancer (ABS210 and ABS211).

TABLE 1

| | β-carotene | |
|---|---|---|
| Vector | (ug/g) (T1 seeds) | Promoter for PSY1 |
| ABS220 | 17.6 | CAMV35S ENH/SB-KAFA B1 PRO |
| ABS221 | 18.3 | CAMV35S ENH/SB-KAFA B1PRO |
| ABS210 | 2.9 | SB-KAFA B1 PRO |
| ABS211 | 6.4 | SB-KAFA B1 PRO |
| TX430 | 0.33 | Non-transgenic |

Agronomic performance of ABS203

The agronomic performance of ABS203 was studied under confined field condition. The yield and germination rate of 13 ABS203 homozygous events with their corresponding nulls and wild type were tested. For each event, 2 reps of 2 row plots were randomly distributed in the field. Twenty seeds were sowed in each 13′ row. Seeds germination data were collect after 4-week sowing. Sorghum plant phenotypes were recorded during plant development. For each row, seeds were harvested in a 3-feet section in the middle of the row to avoid the variations caused on the edge. 1 to 5 sections were harvested in each plot. The total threshed seed weight collected from the 3-feet section was recorded.

The experiment was analyzed as two-way treatment structure (event×segregation) with wild-type check (wt). The data were analyzed in a two-step process. The first step was to analyze all possible treatment combinations (13 event*2 segregation+wt) in a one-way ANOVA. This would allow for the test of any possible combination to be directly compared to any other combination. The second step was to use single degree of freedom contrast statements to estimate the levels of either main effect (event or segregation) or the differences between levels within a main effect. This allowed for the test of a level of an effect against any other level or the wt. Significant differences were deemed when the probability of the difference was less than 0.05.

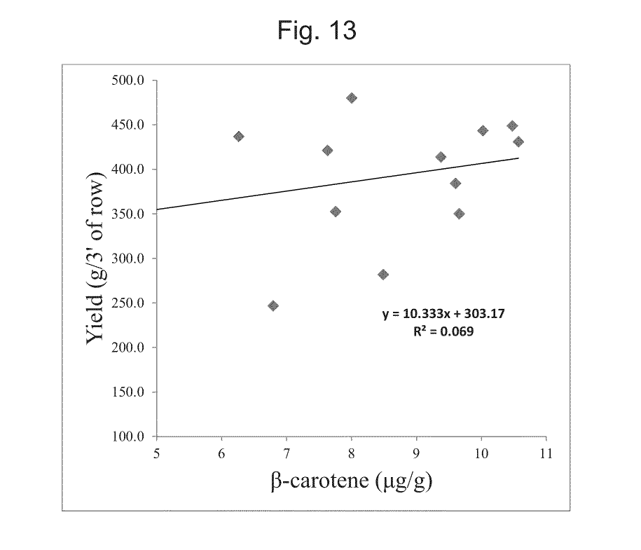

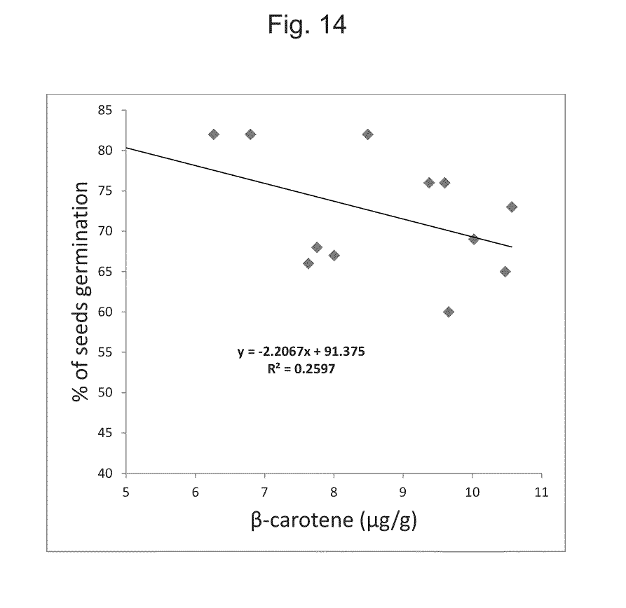

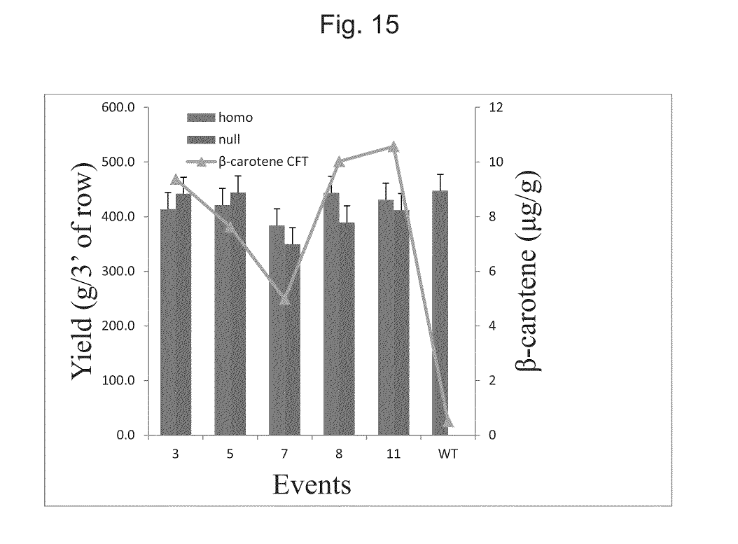

As shown in FIGS. 13, 14, and 15, in both cases, no significant correlation between the β-carotene level and yield (FIG. 13) or between the β-carotene level and germination rate (FIG. 14) were observed (R2<0.5 as indicated in the figures). In other words, there is no yield and germination rate penalties caused by the enhanced β-carotene level. The yield and germinate differences of those 13 events with wild-type are event dependent and most likely due to the random insertion of the transgenes. Therefore, five events with no abnormal phenotypes, no yield and germination rate penalties were identified from these 13 events (FIG. 15).

### Example 31

### Construction of pABS237

The vector pABS237 encoding maize PSY1(v2)+CRTI was constructed using the same approaches as described above. The pABS237 vector comprises transgene cassettes: CAMV35S ENH (−343-90)/SB-AKAF B1 PRO (ALT1)/ZM-PSY1 (ALT1) (V2)/SB-AKAF (B1) TERM//SB-BKAF PRO/PEA SSU TP/CRTI (EU)/SB-BKAF TERM.

### Example 32

### Construction of pABS234

The vector pABS234 encoding maize PSY1(v2)+CRTI+CA-CAP+HV-HGGT was constructed using the same approaches as described above. The pABS234 vector comprises transgene cassettes: CAMV35S ENH (−343-90)/SB-AKAF B1 PRO (ALT1)/ZM-PSY1 (ALT1) (V2)/SB-AKAF (B1) TERM//SB-BKAF PRO/PEA SSU TP/CRTI (EU)/SB-BKAF TERM//GZ-W64A PRO/CA-CAP (GENOMIC)/GZ-W64A TERM//OLE PRO/HV-HGGT/EAP1 TERM.

### Example 33

### Construction of pABS235

The vector pABS235 encoding maize PSY1(v2)+CRTI (MO)+CA-CAP was constructed using the same approaches as described above. The pABS235 vector comprises transgene cassettes: CAMV35S ENH (−343-90)/SB-AKAF B1 PRO (ALT1)/ZM-PSY1 (ALT1) (V2)/SB-AKAF (B1) TERM//SB-BKAF PRO/PEA SSU TP (MO)/CRTI (EU) (MO)/SB-BKAF TERM//GZ-W64A PRO/CA-CAP (GENOMIC)/GZ-W64A TERM.

### Example 34

### Construction of pABS236

The vector pABS236 encoding maize PSY1(v2)+CRTI+CA-CAP+HGGT+SB-PSY3 was constructed using the same approaches as described above. The pABS236 vector comprises transgene cassettes: AMV35S ENH (−343-90)/SB-AKAF B1 PRO (ALT1)/ZM-PSY1 (ALT1) (V2)/SB-AKAF (B1) TERM//SB-BKAF PRO/PEA SSU TP/CRTI (EU)/SB-BKAF TERM//GZ-W64A PRO/CA-CAP (GENOMIC)/GZ-W64A TERM//OLE PRO/HV-HGGT/EAP1 TERM//FL2 PRO (ALT1)/SB-PSY3/FL2 TERM (ALT1).

### Example 35

### Construction of pABX183

The vector pABX183 encoding maize PSY1(v2)+CRTI+CA-CAP+HGGT+ZM-PSY3 is constructed using the same approaches as described above. The pABX183 vector comprises transgene cassettes: CAMV35S ENH (−343-90)/SB-AKAF B1 PRO (ALT1)/ZM-PSY1 (ALT1) (V2)/SB-AKAF (B1) TERM//SB-BKAF PRO/PEA SSU TP/CRT I (EU)/SB-BKAF TERM//GZ-W64A PRO/CA-CAP EXON1 (MO)/CA-CAP INTRON1/CA-CAP EXON2 (MO)/GZ-W64A TERM//OLE PRO/HV-HGGT/EAP1 TERM//FL2 PRO (ALT1) /ZM-PSY3/FL2 TERM (ALT1).

### Example 36

### Construction of pABS239

The vector pABS239 encoding HGGT+CRTB (MO)+PSY(V2)+CRTI (MO) was constructed using the same approaches as described above. The pABS239 vector comprises transgene cassettes: SB-GKAF PRO/HV-HGGT/SB-GKAF TERM (MOD1)//FL2 PRO (ALT1)/CS-DPAD TP (MO)/CRT B (PA) (MO)/FL2 TERM (ALT1)//CAMV35S ENH (−343-90)/SB-AKAF B1 PRO (ALT1)/ZM-PSY1 (ALT1) (V2)/SB-AKAF (B1) TERM//SB-BKAF PRO/PEA SSU TP (MO)/CRT I (EU) (MO)/SB-BKAF TERM. CS-DPAD TP (MO) is a maize optimized version of the polynucleotide encoding the transit peptide of delta-4-palmitoyl-ACP desaturase gene of Coriandrum sativum, having the nucleotide sequence of SEQ ID NO: 50, operably linked to CRT B (PA) (MO), which is a maize codon optimized version of the Erwinia uredovora carotenoid biosynthesis gene having the nucleotide sequence of SEQ ID NO: 48.

### Example 37

### Construction of pABS238

The vector pABS238 encoding HGGT+ZM-PSY3+CRTB(MO)+CRTI (MO) was constructed using the same approaches as described above. The pABS238 vector comprises transgene cassettes: SB-GKAF PRO/HV-HGGT/SB-GKAF TERM (MOD1)//FL2 PRO (ALT1)/ZM-PSY3/FL2 TERM (ALT1)//CAMV35S ENH (−343-90)/SB-AKAF B1 PRO (ALT1)/CS-DPAD TP (MO)/CRT B (PA) (MO)/SB-AKAF (B1) TERM//SB-BKAF PRO/PEA SSU TP (MO)/CRT I (EU) (MO)/SB-BKAF TERM.

### Example 38

### Construction of pABS240

The vector pABS240 encoding HGGT+CRTB (MO)+PSY(V2)+CRTI was constructed using the same approaches as described above. The pABS240 vector comprises transgene cassettes: SB-GKAF PRO/HV-HGGT/SB-GKAF TERM (MOD1)//FL2 PRO (ALT1)/CS-DPAD TP (MO)/CRT B (PA) (MO)/FL2 TERM (ALT1)//CAMV35S ENH (−343-90)/SB-AKAF B1 PRO (ALT1)/ZM-PSY1 (ALT1) (V2)/SB-AKAF (B1) TERM//SB-BKAF PRO/PEA SSU TP/CRT I (EU)/SB-BKAF TERM.

### Example 39

### Construction of pABX446

The vector pABX446 encoding CA-CAP+PSY1(V2)+CRTI is constructed using the same approaches as described above. The pABX446 vector comprises transgene cassettes: GZ-W64A PRO/CA-CAP (MO) EXON1/CA-CAP INTRON1/CA-CAP (MO) EXON2/GZ-W64A TERM//CAMV35S ENH (−343-90)/CAMV35S ENH (−343-90)/CAMV35S ENH (−343-90)/SB-AKAF B1 PRO (ALT1)/ZM-PSY1 (ALT1) (V2)/SB-AKAF (B1) TERM//SB-BKAF PRO/PEA SSU TP/CRT I (EU)/SB-BKAF TERM.

### Example 40

### Construction of pABX447

The vector pABX447 encoding HGGT+PSY1(V2)+CRTI is constructed using the same approaches as described above. The pABX447 vector comprises transgene cassettes: SB-GKAF PRO/HV-HGGT/SB-GKAF TERM (MOD1)//CAMV35S ENH (−343-90)/CAMV35S ENH (−343-90)/CAMV35S ENH (−343-90)/SB-AKAF B1 PRO (ALT1)/ZM-PSY1 (ALT1) (V2)/SB-AKAF (B1) TERM//SB-BKAF PRO/PEA SSU TP/CRT I (EU)/SB-BKAF TERM//FL2 PRO (ALT1)/ZM-PSY3/FL2 TERM (ALT1).

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

That which is claimed is:

1. A recombinant DNA molecule, comprising
a first exogenous expression cassette capable of directing production in a plant cell of at least one enzyme in the carotenoid synthesis pathway; and
a second exogenous expression cassette capable of directing production in a plant cell of at least one enzyme in the tocochromanol synthesis pathway.

2. The recombinant DNA molecule of claim 1, wherein the enzyme in the tocochromanol synthesis pathway is a homogentisate geranylgeranyl transferase (HGGT) derived from Hordeum vulgare.

3. The recombinant DNA molecule of claim 1, further comprising a third exogenous expression cassette capable of directing production in the cell of at least one enzyme in the methylerythritol phosphate biosynthesis pathway.

4. The recombinant recombinant DNA molecule of claim 3, wherein the at least one enzyme in the methylerythritol phosphate biosynthesis pathway is D-1-deoxy-xylulose 5-phosphate synthase (DXS) derived from Arabidopsis thaliana.

5. The recombinant DNA molecule of claim 1, wherein the at least one enzyme in the carotenoid synthesis pathway is a phytoene synthase (PSY) is derived from Zea mays.

6. The recombinant DNA molecule of claim 1, wherein the at least one enzyme in the carotenoid synthesis pathway is a phytoene desaturase (carotenoid reductase (CRT)) derived from Erwinia uredovora.

7. The recombinant DNA molecule of claim 6 wherein the carotenoid reductase (CRT) is operably linked with a suitable plastid transit peptide.

8. The recombinant DNA molecule of claim 2, wherein the homogentisate geranylgeranyl transferase (HGGT) is operably linked to a tissue specific promoter.

9. The recombinant DNA molecule of claim 4, wherein the D-1-deoxy-xylulose 5-phosphate synthase is operably linked to a tissue specific promoter.

10. The recombinant DNA molecule of claim 5, wherein the phytoene synthase is operably linked to a tissue specific promoter.

11. The recombinant DNA molecule of claim 5, wherein the phytoene desaturase is operably linked to a tissue specific promoter.

12. An expression vector, comprising
a first recombinant polynucleotide encoding at least one enzyme in the carotenoid synthesis pathway operably linked to at least one regulatory element;
a second recombinant polynucleotide encoding at least one enzyme in the tocochromanol synthesis pathway operably linked to at least one regulatory element.

13. A method of increasing total carotenoid levels and/or increasing carotenoid half-life in a plant, comprising
transforming a plant cell with the recombinant DNA molecule of claim 1; and
selecting a transformed plant that comprises the cells having increased total carotenoid accumulation and increased carotenoid stability compared to a plant cell not having the second exogenous expression cassette.

14. A transgenic plant or progeny thereof, comprising the recombinant polynucleotide molecule of claim 1.

15. A transgenic plant or progeny thereof, comprising the expression vector of claim 12.

16. The transgenic plant or progeny thereof of claim 14, wherein the plant is sorghum.

17. Seed, grain or processed product thereof of the transgenic sorghum plant of claim 16, wherein the seed, grain or processed product thereof has increased carotenoid levels and carotenoid stability compared to a sorghum plant cell not having the second exogenous expression cassette.

18. A method of increasing carotenoid bioavailability in grain, comprising
expressing in a transgenic plant at least one exogenous enzyme in the carotenoid synthesis pathway in a seed specific manner; and
expressing in a transgenic plant at least one exogenous enzyme in the tocochromanol synthesis pathway in a seed specific manner,
wherein the grain has increased carotenoid bioavailability compared to grain not expressing the enzyme in the tocochromanol synthesis pathway in a seed specific manner.

19. The method of claim 18, wherein the enzyme in the tocochromanol synthesis pathway is a homogentisate geranylgeranyl transferase (HGGT) and the enzyme in the carotenoid synthesis pathway is a phytoene synthase (PSY).

20. The method of claim 19, wherein the method further comprises expressing at least one enzyme in the methylerythritol phosphate biosynthesis pathway in a seed specific manner.
